(19) United States
(12) Patent Application Publication
(10) Pub. No.: US 2003/0236259 A1
(43) Pub. Date: Dec. 25, 2003

(54) Novel aryl- and heteroarylpiperazines

(76) Inventors: Rolf Hohlweg, Kvistgaard (DK); Florencio Zaragoza Dorwald, Ballerup (DK); Henrik Stephensen, Farum (DK); Ingrid Pettersson, Malov (DK); Bernd Peschke, Malov (DK)

Correspondence Address: Reza Green, Esq., Novo Nordisk Pharmaceuticals, Inc., 100 College Road West, Princeton, NJ, 08540, US

(21) Appl. No.: 10/383,310

(22) Filed: Mar. 7, 2003

Related U.S. Application Data
(63) Continuation of application No. PCT/DK03/00071, filed on Feb. 5, 2003.
(60) Provisional application No. 60/399,304, filed on Jul. 26, 2002.

(30) Foreign Application Priority Data
Feb. 5, 2002 (DK) PA 2002 00168
Jul. 26, 2002 (DK) PA 2002 01142

(51) Int. Cl. A61K 31/53; A61K 31/501; A61K 31/496; C 07D 4 3/02

(52) U.S. Cl. 514/242; 514/243; 514/252.02; 514/252.14; 514/253.06; 544/182; 544/183; 544/295; 544/238; 544/363

(57) ABSTRACT

Novel aryl- and heteroarylpiperazines, use of these compounds as pharmaceutical compositions, pharmaceutical compositions comprising the compounds, and a method of treatment employing these compounds and compositions. The compounds show a high and selective binding affinity to the histamine H3 receptor indicating histamine H3 receptor antagonistic, inverse agonistic or agonistic activity. As a result, the compounds are useful for the treatment of diseases and disorders related to the histamine H3 receptor.

### FIELD OF THE INVENTION

The present invention relates to novel aryl- and heteroarylpiperazines, to the use of these compounds as pharmaceutical compositions, to pharmaceutical compositions comprising the compounds, and to a method of treatment employing these compounds and compositions. The present compounds show a high and selective binding affinity to the histamine H3 receptor indicating histamine H3 receptor antagonistic, inverse agonistic or agonistic activity. As a result, the compounds are useful for the treatment of diseases and disorders related to the histamine H3 receptor.

### BACKGROUND OF THE INVENTION

The existence of the histamine H3 receptor has been known for several years and the receptor is of current interest for the development of new medicaments. Recently, the human histamine H3 receptor has been cloned. The histamine H3 receptor is a presynaptic autoreceptor located both in the central and the peripheral nervous system, the skin and in organs such as the lung, the intestine, probably the spleen and the gastrointestinal tract. Recent evidence suggests that the H3 receptor shows intrinsic, constitutive activity, in vitro as well as in vivo (ie it is active in the absence of an agonist. Compounds acting as inverse agonists can inhibit this activity. The histamine H3 receptor has been demonstrated to regulate the release of histamine and also of other neurotransmitters such as serotonin and acetylcholine. A histamine H3 receptor antagonist or inverse agonist would therefore be expected to increase the release of these neurotransmitters in the brain. A histamine H3 receptor agonist, on the contrary, leads to an inhibition of the biosynthesis of histamine and an inhibition of the release of histamine and also of other neurotransmitters such as serotonin and acetylcholine. These findings suggest that histamine H3 receptor agonists, inverse agonists and antagonists could be important mediators of neuronal activity. Accordingly, the histamine H3 receptor is an important target for new therapeutics.

Compounds similar to the compounds of the present invention have previously been disclosed, cf. J. Med. Chem. 1999, 42, 336, J. Med. Chem. 1992, 35, 2369, DE 2804096, J. Org. Chem. 1996, 61, 3849, Bull. Soc. Chim. Fr. 1969, 319, WO 00/66578, WO 99/21845, and J. Med. Chem. 1968, 11(6), 1144-1150. However, these references neither disclose nor suggest that these compounds may have a histamine H3 receptor antagonistic or agonistic activity.

Several publications disclose the preparation and use of histamine H3 agonists and antagonists. Most of these are imidazole derivatives. However, recently some imidazole-free ligands of the histamine H3 receptor have been described (see e.g. Linney et al., J. Med. Chem. 2000, 43, 2362-2370; U.S. Pat. No. 6,316,475, WO 01/66534 and WO 01/74810). However, these compounds differ structurally from the present compounds.

In view of the art's interest in histamine H3 receptor agonists, inverse agonists and antagonists, novel compounds which interact with the histamine H3 receptor would be a highly desirable contribution to the art. The present invention provides such a contribution to the art being based on the finding that a novel class of aryl- and heteroarylpiperazines has a high and specific affinity to the histamine H3 receptor.

Due to their interaction with the histamine H3 receptor, the present compounds are useful in the treatment of a wide range of conditions and disorders in which an interaction with the histamine H3 receptor is beneficial. Thus, the compounds may find use eg in the treatment of diseases of the central nervous system, the peripheral nervous system, the cardiovascular system, the pulmonary system, the gastrointestinal system and the endocrinological system.

### DEFINITIONS

In the structural formulae given herein and throughout the present specification, the following terms have the indicated meaning:

The term “halogen” means F, Cl, Br or I.

The term “alkyl” as used herein represents a saturated, branched or straight hydrocarbon group having the indicated number of carbon atoms. Thus, the terms “C₁₋₃-alkyl”, “C₁₋₈-alkyl” and “C₁₋₁₀-alkyl” as used herein represent saturated, branched or straight hydrocarbon groups having from 1 to 3 carbon atoms, 1 to 8 carbon atoms and from 1 to 10 carbon atoms, respectively. Typical alkyl groups include, but are not limited to, methyl, ethyl, n-propyl, isopropyl, butyl, isobutyl, sec-butyl, tert-butyl, pentyl, hexyl and the like.

The term “alkenyl” as used herein represents a branched or straight hydrocarbon group having the indicated number of carbon atoms and at least one double bond. Thus, the terms “C₂₋₈-alkenyl” and “C₂₋₁₀-alkenyl” as used herein represents a branched or straight hydrocarbon group having from 2 to 8 carbon atoms, and from 2 to 10 carbon atoms respectively, and at least one double bond Examples of such groups include, but are not limited to, ethenyl, 1-propenyl, 2-propenyl, allyl, iso-propenyl, 1,3-butadienyl, 1-butenyl, 2-butenyl, 1-pentenyl, 2-pentenyl, 1-hexenyl, 2-hexenyl, 1-heptenyl, 2-heptenyl, 1-octenyl, 2-octenyl, 2-nonenyl, 2-decenyl and the like.

The term “alkynyl” as used herein represents a branched or straight hydrocarbon group having the indicated number of carbon atoms and at least one triple bond. Thus, the term “C₂₋₈-alkynyl” as used herein represents a branched or straight hydrocarbon group having from 2 to 8 carbon atoms and at least one triple bond. Examples of such groups include, but are not limited to, ethynyl, 1-propynyl, 2-propynyl, 1-butynyl, 2-butynyl, 1-pentynyl, 2-pentynyl, 1-hexynyl, 2-hexynyl, 2-heptynyl, 1-octynyl, 2-octynyl and the like.

The term “branched C₄₋₆-alkyl” as used herein represents a saturated, branched hydrocarbon group having from 4 to 6 carbon atoms. Typical branched C₄₋₈-alkyl groups include, but are not limited to, 1-methylpropyl, tert-butyl, 1-ethylpropyl, 1,1-(dimethyl)propyl, isopentyl 1-ethylbutyl, 1,1-(dimethyl)butyl, 1,1-(dimethyl)pentyl, 1-ethylpentyl, 1,1-(dimethyl)hexyl, 1-ethylhexyl and the like.

The term “branched C₄₋₆-alkenyl” as used herein represents a branched hydrocarbon group having from 4 to 6 carbon atoms and at least one double bond. Typical branched C₄₋₆-alkenyl groups include, but are not limited to, 1-ethylprop-2-enyl, 1,1-(dimethyl)prop-2-enyl, 1-ethylbut-3-enyl, 1,1-(dimethyl)but-2-enyl, 1,1-(dimethyl)pent-3-enyl, 1-ethylpent-2-enyl, 1,1-(dimethyl)pent-3-enyl, 1,1-(dimethyl)hex-3-enyl, 1-ethylhex-4-enyl and the like.

The term “branched C₄₋₆-alkynyl” as used herein represents a branched hydrocarbon group having from 4 to 6 carbon atoms and at least one triple bond. Typical branched C₄₋₆-alkynyl groups include, but are not limited to, 1-ethylprop-2-ynyl, 1,1-(dimethyl)prop-2-ynyl, 1-ethylbut-3-ynyl, 1,1-(dimethyl)but-2-ynyl, 1,1-(dimethyl)pent-3-ynyl, 1-ethylpent-2-ynyl, 1,1-(dimethyl)pent-3-ynyl, 1,1-(dimethyl)hex-3-ynyl, 1-ethylhex-4-ynyl and the like.

The term “C₁₋₆-alkoxy” as used herein refers to the radical —O—C₁₋₆-alkyl, wherein C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “C₂₋₁₀-alkanoyl” as used herein refers to the radical —C(═O)C₁₋₉-alkyl, wherein C₁₋₉-alkyl represents a saturated, branched or straight hydrocarbon groups having from 1 to 9 carbon atoms. Representative examples are acetyl, propionyl, butanoyl, pentanoyl, hexanoyl, heptanoyl, octanoyl, nonanoyl, decanoyl and the like.

The term “C₁₋₆-alkylamino” as used herein refers to the radical —NH—C₁₋₆-alkyl, wherein C₁₋₆-alkyl is as defined above. Representative examples are methylamino, ethylamino, isopropylamino, n-propylamino, butylamino, pentylamino, hexylamino and the like.

The term “di-C₁₋₆-alkylamino” as used herein refers to the radical —N(C₁₋₆-alkyl)₂, wherein C₁₋₆-alky is as defined above. It should be understood that the C₁₋₆-alkyl groups may be the same or different. Representative examples are dimethylamino, methylethylamino, diethylamino, diisopropylamino, di-n-propylamino, dibutylamino, dipentylamino, dihexylamino and the like.

The term “C₃₋₅-cycloalkyl” as used herein represents a monocyclic, carbocyclic group having from from 3 to 8 carbon atoms. Representative examples are cyclopropyl, cyclobutyl, cyclopentyl and the like.

In the same way the terms “C₃₋₆-cycloalkyl” and “C₃₋₈-cycloalkyl” as used herein represent monocyclic, carbocyclic groups having from 3 to 6 carbon atoms and from 3 to 8 carbon atoms, respectively.

The term “C₃₋₇-cycloalkenyl” as used herein represents a monocyclic, carbocyclic, non-aromatic group having from 3 to 7 carbon atoms and at least one double bond. Representative examples are cyclopropenyl, cyclobutenyl, cyclopentenyl and the like.

In the same way the term “C₃₋₆-cycloalkenyl” represents a monocyclic, carbocyclic, non-aromatic group having from 3 to 6 carbon atoms and at least one double bond.

The term “C₃₋₆-cycloalkyl-C₁₋₃-alkyl” as used herein refers to the radical —C₁₋₃-alkyl-C₃₋₆-cycloalkyl where C₃₋₆-cycloalkyl and C₁₋₃-alkyl are as defined above.

The term “C₃₋₆-cycloalkenyl-C₁₋₃-alkyl” as used herein refers to the radical —C₁₋₃-alkyl-C₃₋₆-cycloalkenyl where C₃₋₆-cycloalkenyl and C₁₋₃-alkyl are as defined above.

The term “C₃₋₈-cycloalkyloxy” as used herein refers to the radical —O—C₃₋₈-cycloalkyl where C₃₋₈-cycloalkyl is as defined above. Representative examples are cyclopropyloxy, cyclobutyloxy, cyclopentyloxy, cyclohexyloxy, cycloheptyloxy, cyclooctyloxy and the like.

The term “C₄₋₉-cycloalkanoyl” as used herein refers to the radical —C(═O)—C₃₋₈-cycloalkyl where C₃₋₈-cycloalkyl is as defined above. Representative examples are cyclopropylcarbonyl, cyclobutylcarbonyl, cyclopentylcarbonyl, cyclohexylcarbonyl, cycloheptylcarbonyl, cyclooctylcarbonyl and the like.

The term “C₁₋₆-alkylsulfonyl” as used herein refers to the radical —S(═O)₂—C₁₋₆-alkyl, wherein C₁₋₆-alkyl is as defined above. Representative examples are methylsulfonyl, ethylsulfonyl, isopropylsulfonyl, n-propylsulfonyl, butylsulfonyl, pentylsulfonyl and the like.

The term “C₁₋₆-alkylsulfanyl” as used herein refers to the radical —S—C₁₋₆-alkyl, wherein C₁₋₆-alkyl is as defined above. Representative examples are methylsulfanyl, ethylsulfanyl, isopropylsulfanyl, n-propylsulfanyl, butylsulfanyl, pentylsulfanyl and the like.

The term “C₃₋₈-heterocyclyl” as used herein refers to a saturated 3 to 8 membered monocyclic ring containing one or more heteroatoms selected from nitrogen, oxygen and sulphur. Representative examples are aziridinyl, pyrrolidinyl, piperidinyl, morpholinyl, piperazinyl, tetrahydrofuranyl and the like.

The term “C₄₋₉-heterocycloalkanoyl” as used herein refers to the radical —C(═O)—C₃₋₈-heterocyclyl where C₃₋₈-heterocyclyl is as defined above. Representative examples are aziridinylcarbonyl, pyrrolidinylcarbonyl, piperidinylcarbonyl, morpholinylcarbonyl, piperazinylcarbonyl, tetrahydrofuranylcarbonyl and the like.

The term “aryl” as used herein is intended to include carbocyclic aromatic ring systems such as phenyl, biphenylyl, naphthyl, anthracenyl, phenanthrenyl, fluorenyl, indenyl, pentalenyl, azulenyl and the like. Aryl is also intended to include the partially hydrogenated derivatives of the carbocyclic systems enumerated above. Non-limiting examples of such partially hydrogenated derivatives are 1,2,3,4-tetrahydronaphthyl, 1,4-dihydronaphthyl and the like.

The term “aryloxy” as used herein refers to the radical —O-aryl, wherein aryl is as defined above. Non-limiting examples are phenoxy, naphthoxy, anthracenyloxy, phenantrenyloxy, fluorenyloxy, indenyloxy and the like.

The term “aroyl” as used herein refers to the radical —C(═O)-aryl, wherein aryl is as defined above. Non-limiting examples are benzoyl, naphthoyl, anthracenylcarbonyl, phenantrenylcarbonyl, fluorenylcarbonyl, indenylcarbonyl and the like.

The term “arylamino” as used herein refers to the radical —NH-aryl, wherein aryl is as defined above. Non-limiting examples are phenylamino, naphthylamino, anthracenylamino, phenantrenylamino, fluorenylamino, indenylamino and the like.

The term “heteroaryl” as used herein is intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzothiazolyl, benzoisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indanyl, indolinyl, oxazolidinyl, oxazolinyl, oxazepinyl and the like.

The term “heteroaryloxy” as used herein refers to the radical —O-heteroaryl, wherein heteroaryl is as defined above.

The term “heteroaroyl” as used herein refers to the radical —C(═O)-heteroaryl, wherein heteroaryl is as defined above.

The term “heteroarylamino” as used herein refers to the radical —NH-heteroaryl, wherein heteroaryl is as defined above.

Certain of the above defined terms may occur more than once in the structural formulae, and upon such occurrence each term shall be defined independently of the other.

“Aryl-C₁₋₆-alkyl”, “aryl-C₁₋₆-alkoxy” etc. mean C₁₋₆-alkyl or C₁₋₆-alkoxy as defined above, substituted by aryl as defined above, for example:

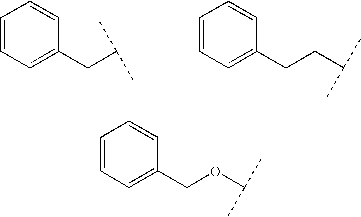

The term “optionally substituted” as used herein means that the groups in question are either unsubstituted or substituted with one or more of the substituents specified. When the groups in question are substituted with more than one substituent the substituents may be the same or different.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The present invention relates to a compound of the general formula (I):

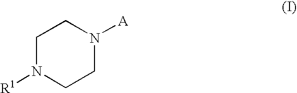

wherein

(i) R¹ represents

branched C₄₋₈-alkyl, branched C₄₋₈-alkenyl or branched C₄₋₈-alkynyl, which may optionally be substituted with one or more halogen substituents,

C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted in any position with one or more halogen substituents, and

A represents

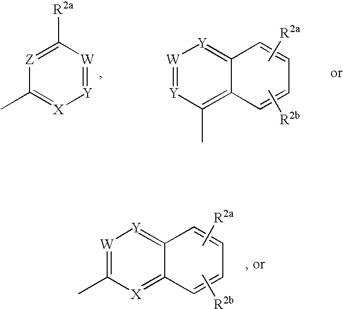

(ii) R¹ represents ethyl, n-propyl or isopropyl, and

A represents

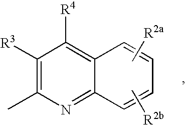

Z and X independently represent —N═, —CH═, —CF═ or —C(CF₃)═,

W represents —N═ or —CR³═,

Y represents —N═ or —CR⁴═,

R^{2a}, R^{2b}, R³, and R⁴ independently represent

hydrogen, halogen, hydroxy, trifluoromethyl, trifluoromethoxy, C-₁₋₁₀-alkyl, C₂₋₁₀-alkenyl, C₃₋₈-cycloalkyl, C₁₋₆-alkoxy, aryl-C₁₋₆-alkyl, amino, C₁₋₆-alkylamino, di-C₁₋₆-alkylamino, C₃₋₈-cycloalkyloxy, cyano, nitro, C₁₋₆-alkylsulfanyl, C₁₋₆-alkylsulfonyl or —C(═O)NR^{4a}R^{4b}, wherein R^{4a} and R^{4b} independently are hydrogen, C₁₋₆-alkyl or aryl-C₁₋₆-alkyl,

C₂₋₁₀-alkanoyl, C₄₋₉-cycloalkanoyl, C₃₋₈-heterocyclyl or C₄₋₉-heterocycloalkanoyl, which may optionally be substituted in any position with one or more substituents selected from aryl, heteroaryl, C₃₋₈-cycloalkyl, halogen, trifluoromethyl, trifluoromethoxy and C₁₋₆-alkoxy,

aryl, aryl-C₁₋₆-alkyl, aryl-C₁₋₆-alkoxy or heteroaryl, which may optionally be substituted with one or more substituents selected from halogen, hydroxy, trifluoromethyl, trifluoromethoxy, C₁₋₆-alkoxy, C₁₋₆-alkyl, amino, C₁₋₆-alkylamino, di-C₁₋₆-alkylamino, cyano, aryl, heteroaryl and C₃₋₈-cycloalkyl,

aroyl, heteroaroyl, aryloxy, heteroaryloxy, arylamino or heteroarylamino, which may optionally be substituted with one or more substituents selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,

or two of R^{2a}, R^{2b}, R³, and R⁴ in adjacent positions together form a C₁₋₆-alkylene bridge,

with the proviso that the compound must not be

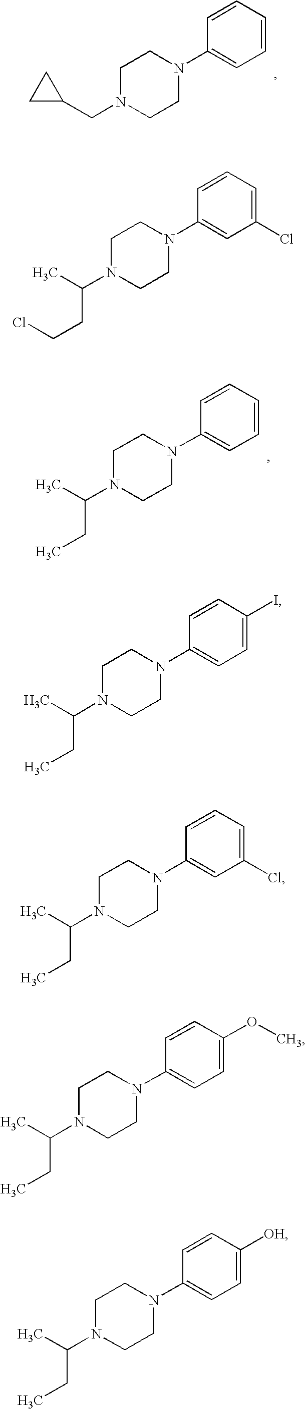

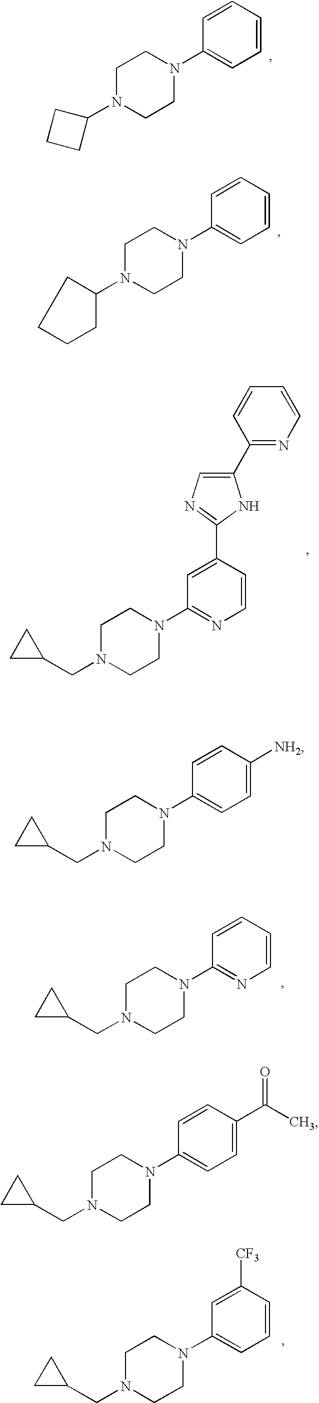

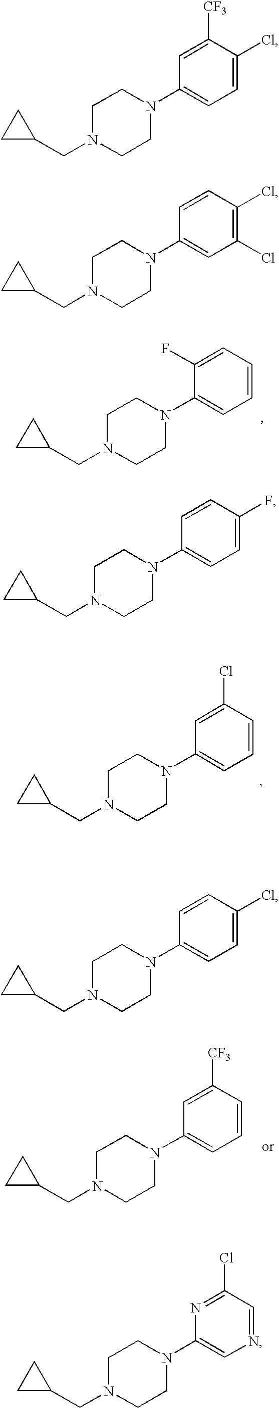

as well as any diastereomer or enantiomer or tautomeric form thereof including mixtures of these or a pharmaceutically acceptable salt thereof.

In one embodiment, R¹ is branched C4-8-alkyl, C3-5-cycloalkyl or C3-6-cycloalkyl-C1-3-alkyl, which may optionally be substituted with one or more halogen substituents.

In another embodiment, R¹ is branched C4-8-alkyl, C3-5-cycloalkyl or C3-6-cycloalkyl-C1-3-alkyl.

In another embodiment, R¹ is 1,1-(dimethyl)propyl, 1-ethylpropyl, cyclopropylmethyl, cyclopropyl, cyclobutyl, cyclopentyl or 1-cyclopropyl-1-methylethyl.

In another embodiment, R¹ is 1-ethylpropyl, cyclopropylmethyl, cyclopropyl or cyclopentyl.

In another embodiment, R¹ is branched C4-8-alkyl or C3-5-cycloalkyl, which may optionally be substituted with one or more halogen substituents.

In another embodiment, R¹ is branched C4-8-alkyl or C3-5-cycloalkyl.

In another embodiment, R¹ is 1-ethylpropyl, cyclopropyl or cyclopentyl.

In another embodiment, R¹ is isopropyl.

In another embodiment, A is

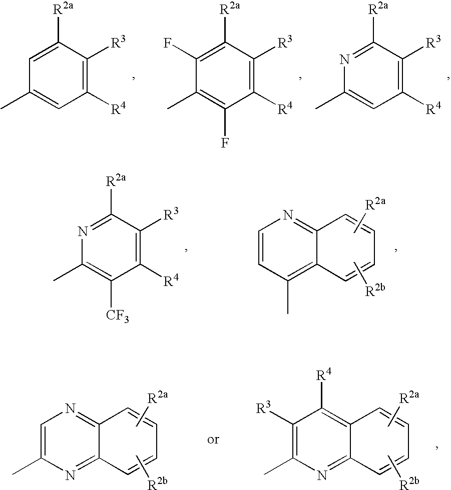

wherein R^{2a}, R^{2b}, R³ and R⁴ are as defined for formula (I)..

In another embodiment, A is

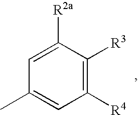

wherein R^{2a}, R³ and R⁴ are as defined for formula (I).

In another embodiment, A is

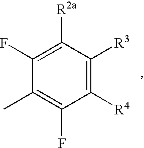

wherein R^{2a}, R³ and R⁴ are as defined for formula (I).

In another embodiment, A is

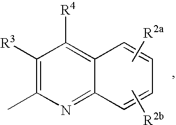

wherein R^{2a}, R^{2b}, R³ and R⁴ are as defined for formula (I).

In another embodiment, R^{2a}, R^{2b}, R³ and R⁴ are independently selected from

hydrogen, hydroxy, halogen, trifluoromethyl, trifluoromethoxy, C2-10-alkanoyl, C4-9-cycloalkanoyl or C4-9-heterocycloalkanoyl, or

aryl-C₁₋₆-alkyl, aryl-C₁₋₆-alkoxy or aroyl, which may optionally be substituted as defined for formula (I).

In another embodiment, R^{2a}, R^{2b}, R³ and R⁴ are independently selected from

hydrogen, hydroxy, halogen, trifluoromethyl, trifluoromethoxy, C2-10-alkanoyl, C4-9-cycloalkanoyl or C4-9-heterocycloalkanoyl,

phenyl-C₁₋₆-alkyl, phenyl-C₁₋₆-alkoxy or benzoyl, which may optionally be substituted with one or two substituents selected from halogen and C₁₋₆-alkoxy.

In another embodiment, R^{2a}, R^{2b} and R⁴ are hydrogen and R³ is different from hydrogen.

In another embodiment, R³ is halogen, trifluoromethyl or trifluoromethoxy.

In another embodiment, the invention relates to a compound of the general formula (I₁):

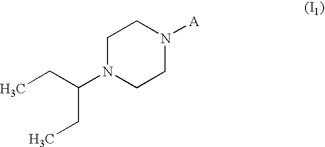

wherein A is as defined for formula (I) or in any one of the above embodiments.

In another embodiment, the invention relates to a compound of the general formula (I₂):

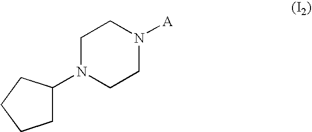

wherein A is as defined for formula (I) or in any one of the above embodiments.

In another embodiment, the invention relates of the general formula (I₃):

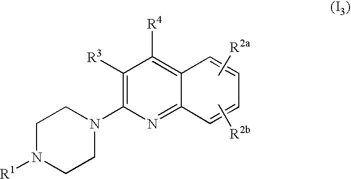

wherein R¹ is

branched C₄₋₈-alkyl, branched C₄₋₈-alkenyl or branched C₄₋₈-alkynyl, which may optionally be substituted with one or more halogen substituents,

C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted in any position with one or more halogen substituents,

ethyl, n-propyl or isopropyl,

R^{2a}, R^{2b}, R³ and R⁴ are as defined for formula (I).

In another embodiment the present invention relates to a compound selected from

4-(4-cyclopentylpiperazin-1-yl)phenol,

1-cyclopentyl-4-[4-(4-fluorobenzyloxy)phenyl]piperazine,

1-(3-chlorophenyl)-4-cyclopentylpiperazine,

1-[4-(4-cyclopentylpiperazin-1-yl)phenyl]ethanone,

1-(3,4-dichlorophenyl)-4-(1-ethylpropyl)piperazine,

{4-[4-(1-ethylpropyl)piperazin-1-yl]phenyl}phenylmethanone,

1-(4-benzylphenyl)-4-(1-ethylpropyl)piperazine,

cyclopropyl-{4-[4-(1-ethylpropyl)piperazin-1-yl]phenyl}methanone,

(2-chlorophenyl)-{4-[4-(1-ethylpropyl)piperazin-1-yl]phenyl}methanone,

{4-[4-(1-Ethylpropyl)piperazin-1-yl]phenyl}-(4-fluorophenyl)methanone,

1-cyclopentyl-4-(6-trifluoromethylpyridin-2-yl)piperazine,

1-cyclopentyl-4-(5-trifluoromethylpyridin-2-yl)piperazine,

1-cyclopentyl-4-(3-trifluoromethylpyridin-2-yl)piperazine,

2-[4-(1-ethylpropyl)piperazin-1-yl]quinoline,

7-chloro-4-[4-(1-ethylpropyl)piperazin-1-yl]quinoline,

[4-(4-cyclopentylpiperazin-1-yl)phenyl]-(3,4-dimethoxyphenyl)methanone,

[4-(4-cyclopentylpiperazin-1-yl)-3,5-difluorophenyl]phenylmethanone,

2-(4-cyclopentylpiperazin-1-yl)quinoxaline,

2-(4-cyclopropylmethylpiperazin-1-yl)quinoxaline,

[6-(4-cyclopentylpiperazin-1-yl)pyridin-3-yl]piperidin-1-ylmethanone,

2-(4-cyclopentylpiperazin-1-yl)quinoline,

2-(4-cyclopentylpiperazin-1-yl)-7-methoxy-3-(4-methoxyphenyl)quinoline,

{6-[4-(1-cyclopropyl-1-methylethyl)piperazin-1-yl]pyridin-3-yl}phenylmethanone,

{4-[4-(1-cyclopropyl-1-methylethyl)piperazin-1-yl]-3,5-difluorophenyl}phenylmethanone,

{4-[4-(1-cyclopropyl-1-methylethyl)piperazin-1-yl]-3,5-difluorophenyl}phenylmethanol

[4-(4-cyclopropylmethylpiperazin-1-yl)-3,5-difluorophenyl]-(4-fluorophenyl)methanone,

{4-[4-(1-ethylpropyl)piperazin-1-yl]-3,5-difluorophenyl}-(4-fluorophenyl)methanone,

2-[4-(1-ethylpropyl)piperazin-1-yl]-6,7-dimethoxyquinoline,

2-[4-(1-ethylpropyl)piperazin-1-yl]-4-trifluoromethylquinoline,

2-(4-cyclopropylmethylpiperazin-1-yl)-6-methoxy-4-trifluoromethylquinoline,

[4-(4-cyclopropylmethylpiperazin-1-yl)-3,5-difluorophenyl]phenylmethanone,

[4-(4-cyclopropylmethylpiperazin-1-yl)-3,5-difluorophenyl]-(3-fluoro-4-methoxyphenyl)-methanone,

{6-[4-(1-ethylpropyl)piperazin-1-yl]pyridin-3-yl}phenylmethanone,

{2-[4-(1-ethylpropyl)piperazin-1-yl]pyridin-4-yl}phenylmethanone,

{4-[4-(1-ethylpropyl)piperazin-1-yl]phenyl}-(4-hydroxyphenyl)methanone,

{6-[4-(1-ethylpropyl)piperazin-1-yl]pyridin-3-yl}piperidin-1-yl-methanone,

N-benzyl-6-[4-(1-ethylpropyl)piperazin-1-yl]-N-methylnicotinamide,

2-[4-(1-ethylpropyl)piperazin-1-yl]-6-methoxyquinoline,

6-[4-(1-ethylpropyl)piperazin-1-yl]-N-methyl-N-phenylnicotinamide,

{6-[4-(1-ethylpropyl)piperazin-1-yl]pyridin-3-yl}-(4-fluorophenyl)methanone,

2-[4-(1-ethylpropyl)piperazin-1-yl]-4-methylquinoline,

2-[4-(1-ethylpropyl)piperazin-1-yl]-5,6,7,8-tetrahydroquinoline,

2-(4-cyclopropylmethylpiperazin-1-yl)-6-methoxyquinoline,

2-(4-isopropylpiperazin-1-yl)-6-methoxyquinoline,

2-[4-(1-ethylpropyl)piperazin-1-yl]-6-fluoro-4-methylquinoline,

2-(4-cyclopropylpiperazin-1-yl)-6-trifluoromethylquinoline,

2-(4-cyclopropylpiperazin-1-yl)-6-propylquinoline,

2-(4-ethylpiperazin-1-yl)quinoline,

as well as any diastereomer or enantiomer or tautomeric form thereof including mixtures of these or a pharmaceutically acceptable salt thereof.

In another aspect the present invention relates to a compound of the general formula (I″):

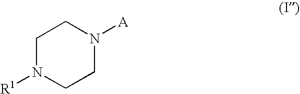

wherein

R¹ represents

branched C₄₋₈-alkyl, branched C₄₋₈-alkenyl or branched C₄₋₈-alkynyl, which may optionally be substituted with one or more halogen substituents,

C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted with one or more halogen substituents,

A represents

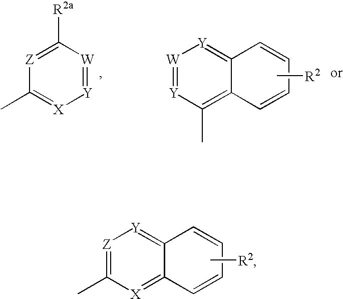

Z and X independently represent —N═, —CH═, —CF═ or —C(CF₃)═,

W represents —N═ or —CR³═,

Y represents —N═ or —CR⁴═,

R², R³, and R⁴ independently represent

hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl-C₁₋₆-alkyl, amino, C₁₋₆-alkylamino, di-C₁₋₆-alkylamino, C₃₋₈-cycloalkyloxy or cyano, or

C₂₋₁₀-alkanoyl or C₄₋₉-cycloalkanoyl, which may optionally be substituted with one or more substituents selected from aryl, heteroaryl, C₃₋₈-cycloalkyl, halogen, trifluoromethyl, trifluoromethoxy and C₁₋₆-alkoxy,

aryl, aryl-C₁₋₆-alkyl, aryl-C₁₋₆-alkoxy or heteroaryl, which may optionally be substituted with one or more substituents selected from halogen, hydroxy, trifluoromethyl, trifluoromethoxy, C₁₋₆-alkoxy, C₁₋₆-alkyl, amino, C₁₋₆-alkylamino, di-C₁₋₆-alkylamino, cyano, aryl, heteroaryl and C₃₋₈-cycloalkyl, or

aroyl, heteroaroyl, aryloxy, heteroaryloxy, arylamino or heteroarylamino, which may optionally be substituted with one or more substituents selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,

with the proviso that the compound must not be

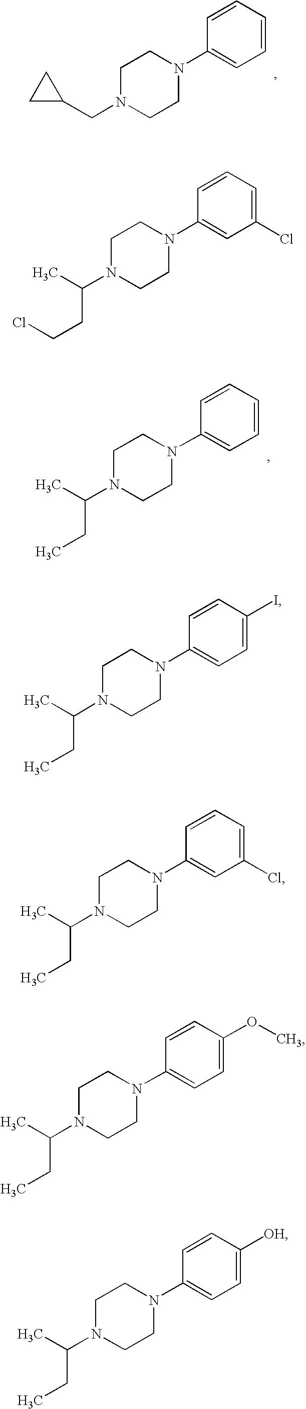

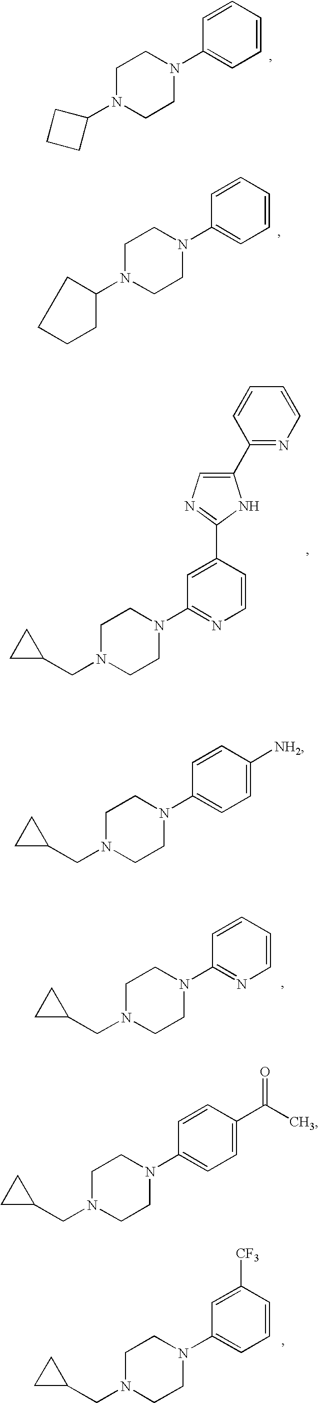

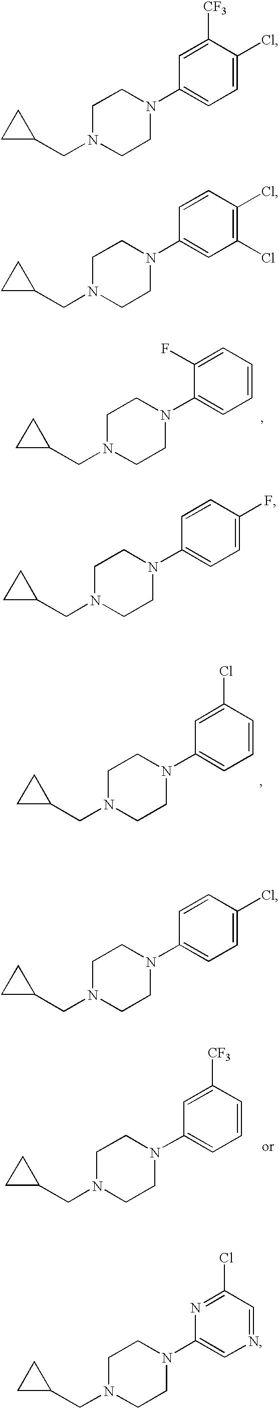

as well as any diastereomer or enantiomer or tautomeric form thereof including mixtures of these or a pharmaceutically acceptable salt thereof.

In one embodiment R¹ is branched C₄₋₈-alkyl, C₃₋₅-cycloalkyl or C₃₋₆-cycloalkyl-C₁₋₃-alkyl, which may optionally be substituted with one or more halogen substituents.

In another embodiment R¹ is branched C₄₋₈-alkyl, C₃₋₅-cycloalkyl or C₃₋₆-cycloalkyl-C₁₋₃-alkyl, such as 1,1-(dimethyl)propyl, 1-ethylpropyl, cyclopropylmethyl, cyclopropyl, cyclobutyl or cyclopentyl, eg 1-ethylpropyl, cyclopropylmethyl or cyclopentyl.

In yet another embodiment R¹ is branched C₄₋₈-alkyl or C₃₋₅-cycloalkyl, which may optionally be substituted with one or more halogen substituents, such as branched C₄₋₈-alkyl or C₃₋₅-cycloalkyl, eg 1-ethylpropyl or cyclopentyl.

In a further embodiment A is

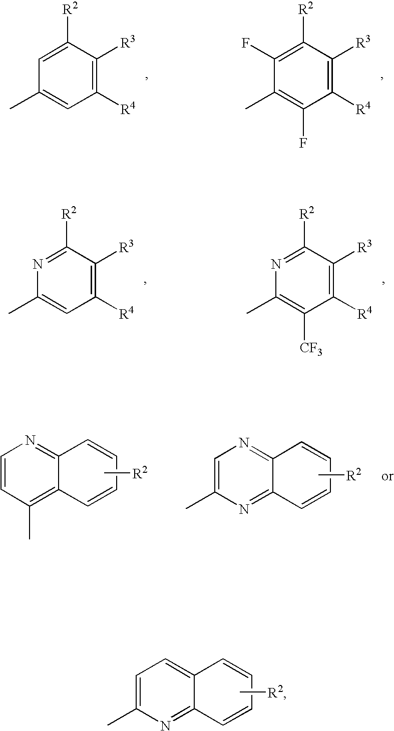

wherein R², R³ and R⁴ are as defined for formula (I″).

In another embodiment A is

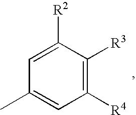

wherein R², R³ and R⁴ are as defined for formula (I″).

In a further embodiment A is

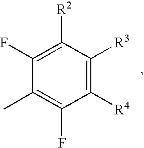

wherein R², R³ and R⁴ are as defined for formula (I″).

In one embodiment R², R³ and R⁴ are independently selected from

hydrogen, hydroxy, halogen, trifluoromethyl, C2-10-alkanoyl or C4-9-cycloalkanoyl, or

aryl-C₁₋₆-alkyl, aryl-C₁₋₆-alkoxy or aroyl, which may optionally be substituted as defined for formula (I″).

In another embodiment R², R³ and R⁴ are independently selected from

hydrogen, hydroxy, halogen, trifluoromethyl, C2-10-alkanoyl or C4-9-cycloalkanoyl,

phenyl-C₁₋₆-alkyl, phenyl-C₁₋₆-alkoxy or benzoyl, which may optionally be substituted with one or two substituents selected from halogen and C₁₋₆-alkoxy.

In still another embodiment R² and R⁴ are both hydrogen and R³ is different from hydrogen.

In yet another embodiment the invention relates to a compound of the general formula (I₁):

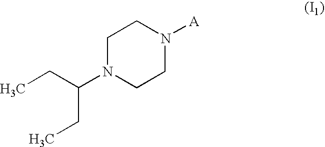

wherein A is as defined for formula (I″) or in any one of the above embodiments.

In still another embodiment the invention relates to a compound of the general formula (I₂):

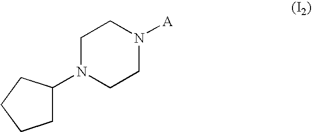

wherein A is as defined for formula (I″) or in any one of the above embodiments.

Accordingly, in another aspect the present invention relates to a compound of the general formula (I) as well as any diastereomer or enantiomer or tautomeric form thereof including mixtures of these or a pharmaceutically acceptable salt thereof for use as a pharmaceutical composition.

The invention also relates to pharmaceutical compositions comprising, as an active ingredient, at least one compound of the formula (I) or any diastereomer or enantiomer or tautomeric form thereof including mixtures of these or a pharmaceutically acceptable salt thereof together with one or more pharmaceutically acceptable carriers or diluents. Furthermore, the invention relates to the use of a compound of the general formula (I′):

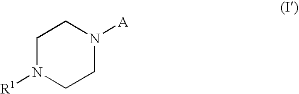

wherein

R¹ represents

C₁₋₈-alkyl, C₂₋₈-alkenyl or C₂₋₈-alkynyl, which may optionally be substituted with one or more halogen substituents,

C₃₋₅-cycloalkyl, C₃₋₇-cycloalkenyl, C₃₋₆-cycloalkyl-C₁₋₃-alkyl or C₃₋₆-cycloalkenyl-C₁₋₃alkyl, which may optionally be substituted with one or more halogen substituents,

A represents

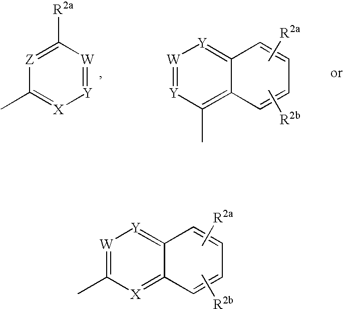

Z and X independently represent —N═, —CH═, —CF═ or —C(CF₃)═,

W represents —N═ or —CR³═,

Y represents —N═ or —CR⁴═,

R^{2a}, R^{2b}, R³, and R⁴ independently represent

hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl-C₁₋₆-alkyl, amino, C₁₋₆-alkylamino, di-C₁₋₆-alkylamino, C₃₋₈-cycloalkyloxy, cyano, nitro, C₁₋₆-alkylsulfanyl, C₁₋₆-alkylsulfonyl, or —C(═O)NR^{4a}R^{4b}, wherein R^{4a} and R^{4b} independently are hydrogen, C₁₋₆-alkyl or aryl-C₁₋₆-alkyl, or

C₂₋₁₀-alkanoyl, C₄₋₉-cycloalkanoyl, C₃₋₈-heterocyclyl or C₄₋₉-heterocycloalkanoyl, which may optionally be substituted in any position with one or more substituents selected from aryl, heteroaryl, C₃₋₈-cycloalkyl, halogen, trifluoromethyl, trifluoromethoxy and C₁₋₆-alkoxy,

aryl, aryl-C₁₋₆-alkyl, aryl-C₁₋₆-alkoxy or heteroaryl, which may optionally be substituted with one or more substituents selected from halogen, hydroxy, trifluoromethyl, trifluoromethoxy, C₁₋₆-alkoxy, C₁₋₆-alkyl, amino, C₁₋₆-alkylamino, di-C₁₋₆-alkylamino, cyano, aryl, heteroaryl and C₃₋₈-cycloalkyl, or

aroyl, heteroaroyl, aryloxy, heteroaryloxy, arylamino or heteroarylamino, which may optionally be substituted with one or more substituents selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,

or two of R^{2a}, R^{2b}, R³, and R⁴ in adjacent positions together form a C₁₋₆-alkylene bridge,

as well as any diastereomer or enantiomer or tautomeric form thereof including mixtures of these or a pharmaceutically acceptable salt thereof for the preparation of a pharmaceutical composition for the treatment of disorders and diseases related to the histamine H3 receptor.

In another aspect the invention provides a compound of the general formula (II):

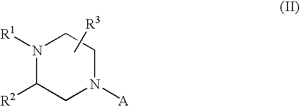

wherein

R² is hydrogen or C₁₋₄-alkyl,

(i) R¹ represents

branched C₄₋₆-alkyl, branched C₄₋₆-alkenyl or branched C₄₋₆-alkynyl with the proviso that R¹ is not isobutyl,

C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl,

R¹ and R² together form a C₃₋₆-alkylene bridge, and

A represents

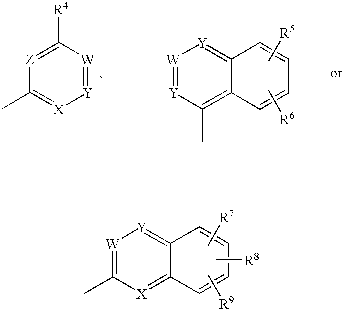

or

(ii) R¹ represents

ethyl, n-propyl or isopropyl,

R¹ and R² together form a C₃₋₆-alkylene bridge, and

A represents

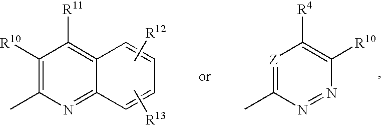

R³ is hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl, aryl-C₁₋₆-alkyl, amino, C₁₋₆-alkylamino, di-C₁₋₆-alkylamino, C₃₋₈-cycloalkyl, C₃₋₈-cycloalkyloxy, cyano, nitro, C₁₋₆-alkylsulfanyl, or C₁₋₆-alkylsulfonyl,

Z and X independently represent —N═, —C(H)═, —C(F)═, —C(Cl)═, —C(CN)═ or —C(CF₃)═,

W represents —N═ or —C(R¹⁰)═,

Y represents —N═ or —C(R¹¹)═,

R⁴, R⁵, R⁶, R⁷, R⁸, R⁹ R¹⁰, R¹¹, R¹² and R¹³ independently represent

hydrogen, halogen, hydroxy, trifluoromethyl, trifluoromethoxy, —SCF₃, amino, cyano, nitro, or —C(═O)NR¹⁴R¹⁵

C₁₋₁₀-alkyl, C₂₋₁₀-alkenyl, C₃₋₈-cycloalkyl, C₁₋₆-alkoxy, C₃₋₈-cycloalkyl-C₁₋₆-alkoxy, C₁₋₆-alkylamino, di-C₁₋₆-alkylamino, C₃₋₈-cycloalkyloxy, C₁₋₆-alkylsulfanyl, C₁₋₆-alkylsulfonyl, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶

aryl, aryl-C₁₋₆-alkyl, aryl-C₁₋₆-alkoxy or heteroaryl, which may optionally be substituted with one or more substituents selected from R¹⁷,

aroyl, heteroaroyl, aryloxy, heteroaryloxy, arylamino or heteroarylamino, which may optionally be substituted with one or more substituents selected from R¹⁸,

or two of R⁵, R⁶, R⁷, R⁸, R⁹, R¹⁰, R¹¹, R¹² and R¹³ in adjacent positions together form a C₁₋₆-alkylene bridge or an —O—C₁₋₆-alkylene-O— bridge,

R¹⁴ and R¹⁵ are independently hydrogen, C₁₋₆-alkyl, aryl-C₁₋₆-alkyl or R¹⁴ and R¹⁵ may together form a C₃₋₆-alkylene bridge

R¹⁶ is independently selected from aryl, heteroaryl, C₃₋₈-cycloalkyl, halogen, trifluoromethyl, trifluoromethoxy, NR¹⁹R²⁰ and C₁₋₆-alkoxy,

R¹⁷ is independently selected from halogen, hydroxy, trifluoromethyl, trifluoromethoxy, C₁₋₁₆-alkoxy, C₁₋₆-alkyl, amino, C₁₋₆-alkylsulfonyl, C₁₋₆-alkylamino, di-C₁₋₆-alkylamino, cyano, aryl, heteroaryl and C₃₋₈-cycloalkyl,

R¹⁸ is independently selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,

R¹⁹ and R²⁰ are independently hydrogen or C₁₋₆-alkyl, R₁₉ and R₂₀ may together form a C₃₋₆ alkylene-bridge

with the proviso that the compound must not be

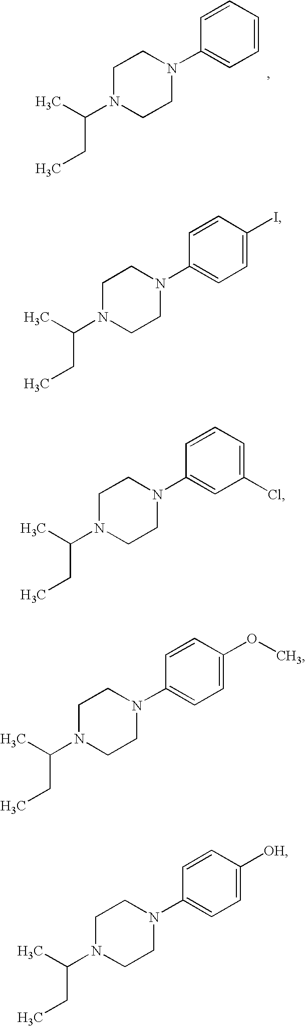

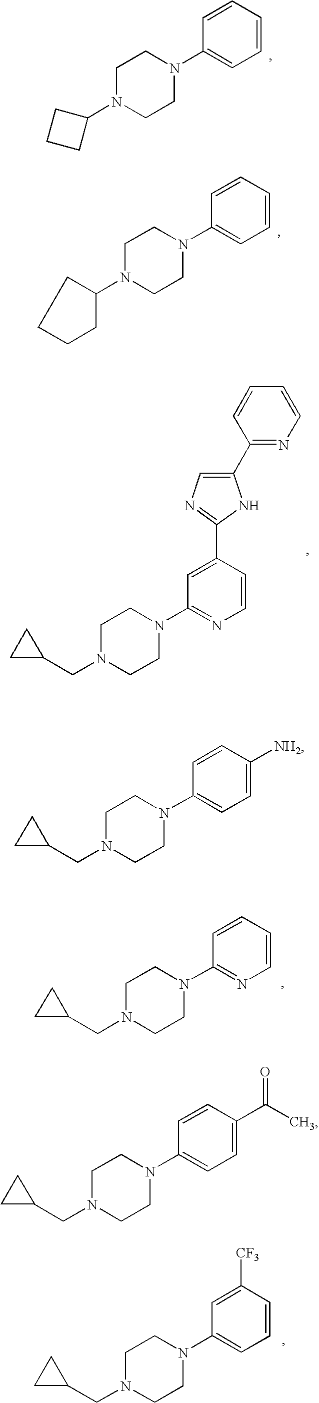

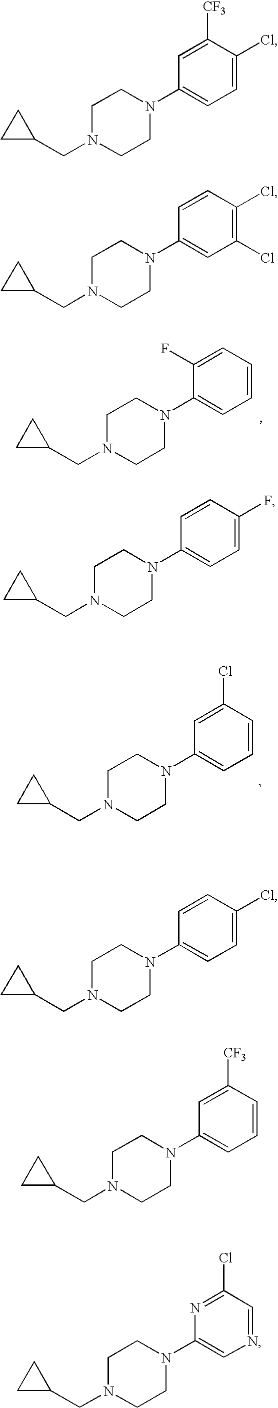

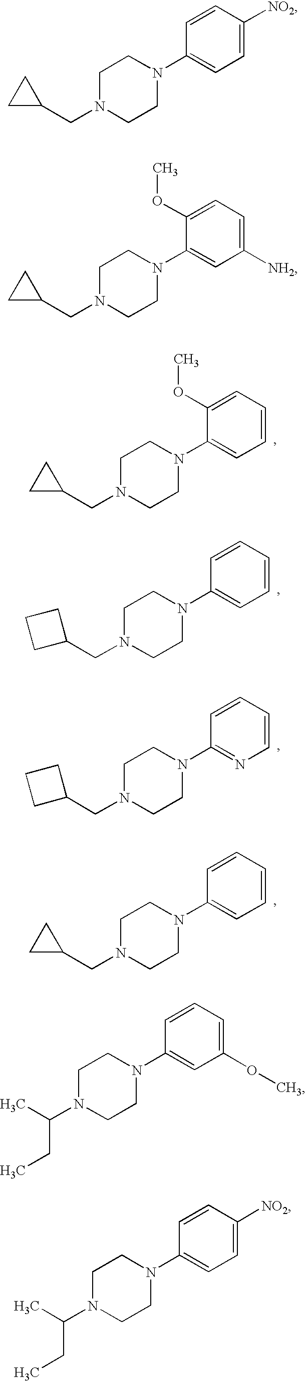

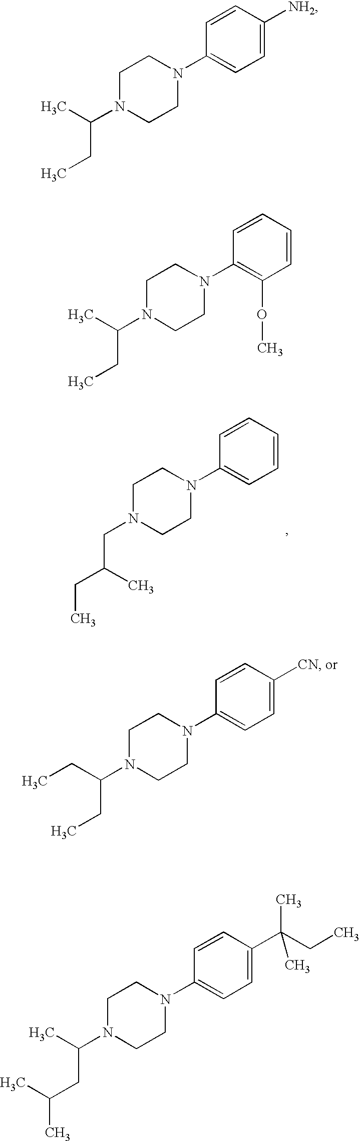

as well as any diastereomer or enantiomer or tautomeric form thereof including mixtures of these or a pharmaceutically acceptable salt thereof.

In another aspect of the invention R¹ is branched C₄₋₆-alkyl, C₃₋₅-cycloalkyl or C₃₋₆-cycloalkyl-C₁₋₃-alkyl with the proviso that R¹ is not isobutyl.

In another aspect of the invention R¹ is 1,1-(dimethyl)propyl, 1-ethylpropyl, cyclopropylmethyl, cyclopropyl, cyclobutyl, cyclopentyl or 1-cyclopropyl-1-methylethyl.

In another aspect of the invention R¹ is 1-ethylpropyl, cyclopropylmethyl, cyclopropyl or cyclopentyl.

In another aspect of the invention R¹ is branched C₄₋₆-alkyl or C₃₋₅-cycloalkyl with the proviso that R¹ is not isobutyl.

In another aspect of the invention R¹ is 1-ethylpropyl, cyclopropyl or cyclopentyl.

In another aspect of the invention Z is —C(H)═, —N═, or —C(F)═.

In another aspect of the invention Z is —C(H)═ or —N═.

In another aspect of the invention Z is —C(H)═.

In another aspect of the invention Z is —N═.

In another aspect of the invention X is —C(H)═, —N═, or —C(F)═.

In another aspect of the invention Z is —C(H)═ or —N═.

In another aspect of the invention Z is —C(H)═.

In another aspect of the invention Z is —N═.

In another aspect of the invention W is —N═.

In another aspect of the invention W is —C(R¹⁰)═.

In another aspect of the invention Y is —N═.

In another aspect of the invention Y is —C(R¹¹)═.

In another aspect of the invention R² is hydrogen.

In another aspect of the invention R² is C₁₋₄-alkyl.

In another aspect of the invention R² is methyl or ethyl.

In another aspect the invention provides compounds of the general formula (III)

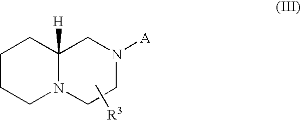

wherein A and R³ are as defined for compounds of general formula (II)

In another aspect of the invention R³ is hydrogen, halogen, hydroxy, trifluoromethyl, trifluoromethoxy, C₁₋₁₀-alkyl, C₁₋₆-alkoxy, aryl, aryl-C₁₋₆-alkyl, amino, C₃₋₈-cycloalkyl, C₃₋₈-cycloalkyloxy, cyano or nitro.

In another aspect of the invention R³ is hydrogen, halogen, hydroxy, trifluoromethyl, C₁₋₁₀-alkyl, C₁₋₆-alkoxy, cyano or nitro.

In another aspect of the invention R³ is hydrogen, halogen, hydroxy, trifluoromethyl, C₁₋₆-alkyl, or cyano.

In another aspect of the invention R³ is hydrogen, halogen, or C₁₋₆-alkyl.

In another aspect of the invention R³ is hydrogen or methyl.

In another aspect of the invention R⁴, R⁵, R⁶, R⁷, R⁸, and R⁹ independently represent

hydrogen, halogen, hydroxy, trifluoromethyl, trifluoromethoxy, —SCF₃, amino, or cyano,

C₁₋₁₀-alkyl, C₃₋₈-cycloalkyl, C₁₋₆-alkoxy, C₃₋₈-cycloalkyloxy, C₂₋₁₀-alkanoyl, C₄₋₉-cycloalkanoyl, C₃₋₈-heterocyclyl or C₄₋₉-heterocycloalkanoyl, which may optionally be substituted with one or more substituents selected from R¹⁶

aryl, aryl-C₁₋₆-alkyl, aryl-C₁₋₆-alkoxy or heteroaryl, which may optionally be substituted with one or more substituents selected from R¹⁷,

aroyl, heteroaroyl, aryloxy, heteroaryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, hydroxy, trifluoromethyl, trifluoromethoxy, —SCF₃, or cyano,

C₁₋₁₀-alkyl, C₁₋₆-alkoxy, C₃₋₈-cycloalkyloxy, which may optionally be substituted with one or more substituents selected from R¹⁶

Aryl or aryl-C₁₋₆-alkyl, which may optionally be substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl or C₁₋₆-alkoxy which may optionally be substituted with one or more substituents selected from R¹⁶

Aryl optionally substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

methyl, ethyl, propyl, isopropyl or C₁₋₆-alkoxy which may optionally be substituted with one or more substituents selected from R¹⁶

Aryl optionally substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl, methoxy, ethoxy or propoxy which may optionally be substituted with one or more substituents selected from R¹⁶

Aryl optionally substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl or methoxy which may optionally be substituted with one or more substituents selected from R¹⁶

Aryl optionally substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl or C₁₋₆-alkoxy which may optionally be substituted with one or more substituents selected from R¹⁶

phenyl optionally substituted with one or more substituents selected from R¹⁷,

Aroyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl or C₁₋₆-alkoxy which may optionally be substituted with one or more substituents selected from R¹⁶

Aryl optionally substituted with one or more substituents selected from R¹⁷,

—C(═O)-phenyl or aryloxy, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R⁴, R⁵, R⁶, R⁷, R⁸, and R⁹ independently represent

hydrogen, halogen, or cyano,

C₁₋₁₀-alkyl or C₁₋₆-alkoxy which may optionally be substituted with one or more substituents selected from R¹⁶

Aryl optionally substituted with one or more substituents selected from R¹⁷,

Aroyl or —O-phenyl, which may optionally be substituted with one or more substituents selected from R¹⁸,

or two of R⁵, R⁶, R⁷, R⁸, R⁹ in adjacent positions together form a C₁₋₆-alkylene bridge or an —O—C₁₋₆-alkylene-O— bridge.

In another aspect of the invention R¹ is ethyl or isopropyl.

In another aspect of the invention R¹ is isopropyl.

In another aspect of the invention R¹ is ethyl

In another aspect of the invention R¹ and R² together form a C₃₋₄-alkylene bridge.

In another aspect of the invention R¹⁰, R¹¹, R¹² and R¹³ independently represent

hydrogen, halogen, hydroxy, trifluoromethyl, trifluoromethoxy, cyano, or —C(═O)NR¹⁴R¹⁵

C₁₋₁₀-alkyl, C₃₋₈-cycloalkyl, C₁₋₆-alkoxy, C₂₋₁₀-alkanoyl, C₄₋₉-cycloalkanoyl,C₃₋₈-heterocyclyl or C₄₋₉-heterocycloalkanoyl, C₄₋₉-heterocycloalkoxy, which may optionally be substituted with one or more substituents selected from R¹⁶

aryl, aryl-C₁₋₆-alkyl, aryl-C₁₋₆-alkoxy or heteroaryl, which may optionally be substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, hydroxy, trifluoromethyl, trifluoromethoxy, cyano, or —C(═O)NR¹⁴R¹⁵

C₁₋₁₀-alkyl, C₃₋₈-cycloalkyl, C₁₋₆-alkoxy, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶

aryl, aryl-C₁₋₆-alkyl, aryl-C₁₋₆-alkoxy or heteroaryl, which may optionally be substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, trifluoromethyl, or —C(═O)NR¹⁴R¹⁵

C₁₋₁₀-alkyl, C₁₋₆-alkoxy, C₂₋₁₀-alkanoyl, C₄₋₉-cycloalkanoyl, C₄₋₉-heterocycloalkanoyl or C₄₋₉-heterocycloalkoxy, which may optionally be substituted with one or more substituents selected from R¹⁶

aryl, aryl-C₁₋₆-alkyl, or aryl-C₁₋₆-alkoxy, which may optionally be substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, trifluoromethyl, or —C(═O)NR¹⁴R¹⁵

C₁₋₁₀-alkyl or C₄₋₉-heterocycloalkanoyl, which may optionally be substituted with one or more substituents selected from R¹⁶

aryl optionally substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, trifluoromethyl, or —C(═O)NR¹⁴R¹⁵

methyl, ethyl,, propyl or C₄₋₉-heterocycloalkanoyl, which may optionally be substituted with one or more substituents selected from R¹⁶

aryl optionally substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, trifluoromethyl, or —C(═O)NR¹⁴R¹⁵

C₁₋₁₀-alkyl, piperidin-alkanoyl or pyrrolidin-alkanoyl, which may optionally be substituted with one or more substituents selected from R¹⁶

aryl optionally substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁰, R¹¹, R¹² and R¹³ independently represent

hydrogen, halogen, trifluoromethyl, or —C(═O)NR¹⁴R¹⁵

C₁₋₁₀-alkyl or C₄₋₉-heterocycloalkanoyl, which may optionally be substituted with one or more substituents selected from R¹⁶

Phenyl optionally substituted with one or more substituents selected from R¹⁷,

aroyl optionally substituted with one or more substituents selected from R¹⁸,

or two of R¹⁰, R¹¹, R¹² and R¹³ in adjacent positions together form a C₁₋₆-alkylene bridge.

In another aspect of the invention R¹⁴ and R¹⁵ are independently methyl, ethyl or benzyl.

In another aspect of the invention R¹⁶ is halogen, trifluoromethyl, trifluoromethoxy and C₁₋₆-alkoxy.

In another aspect of the invention R¹⁷ is halogen, hydroxy, trifluoromethyl, C₁₋₆-alkoxy, C₁₋₆-alkyl, C₁₋₆-alkylsulfonyl, or cyano.

In another aspect of the invention R¹⁷ is halogen, trifluoromethyl, C₁₋₆-alkoxy or C₁₋₆-alkylsulfonyl.

In another aspect of the invention R¹⁸ is C₁₋₁₀-alkyl, halogen, trifluoromethyl, C₁₋₆-alkoxy, cyano, amino and hydroxy.

In another aspect of the invention R¹⁸ is halogen, C₁₋₆-alkoxy and hydroxy.

In another aspect the invention provides the use of a compound according to formula (II) or (III) as a pharmaceutical composition. The pharmaceutical composition may in another aspect of the invention comprise, as an active ingredient, at least one compound according to formula (II) or (III) together with one or more pharmaceutically acceptable carriers or excipients. In another aspect the invention provides such a pharmaceutical composition in unit dosage form, comprising from about 0.05 mg to about 1000 mg, preferably from about 0.1 mg to about 500 mg and especially preferred from about 0.5 mg to about 200 mg of the compound according to formula (II) or (III)

In another aspect the invention provides the use of a compound of the general formula (II′):

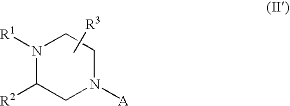

wherein

R² is hydrogen or C₁₋₄-alkyl,

R¹ represents

C₁₋₈-alkyl, C₂₋₈-alkenyl or C₂₋₈-alkynyl, which may optionally be substituted with one or more halogen substituents,

C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted with one or more halogen substituents,

R¹ and R² together form a C₃₋₆-alkylene bridge

A represents

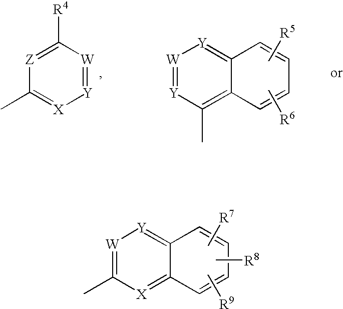

R³ is hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl, aryl-C₁₋₆-alkyl, amino, C₁₋₆-alkylamino, di-C₁₋₆-alkylamino, C₃₋₈-cycloalkyl, C₃₋₈-cycloalkyloxy, cyano, nitro, C₁₋₆-alkylsulfanyl, or C₁₋₆-alkylsulfonyl,

Z and X independently represent —N═, —C(H)═, —C(F)═, —C(Cl)═, —C(CN)═ or —C(CF₃)═,

W represents —N═ or —C(R¹⁰)═,

Y represents —N═ or —C(R¹¹)═,

R⁴, R⁵, R⁶, R⁷, R⁸, R⁹ R¹⁰, R¹¹, R¹² and R¹³ independently represent

hydrogen, halogen, hydroxy, trifluoromethyl, trifluoromethoxy, —SCF₃, amino, cyano, nitro, or —C(═O)NR¹⁴R¹⁵

C₁₋₁₀-alkyl, C₂₋₁₀-alkenyl, C₃₋₈-cycloalkyl, C₁₋₆-alkoxy, C₃₋₈-cycloalkyl-C₁₋₆-alkoxy, C₁₋₆-alkylamino, di-C₁₋₆-alkylamino, C₃₋₈-cycloalkyloxy, C₁₋₆-alkylsulfanyl, C₁₋₆-alkylsulfonyl, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶

aryl, aryl-C₁₋₆-alkyl, aryl-C₁₋₆-alkoxy or heteroaryl, which may optionally be substituted with one or more substituents selected from R¹⁷,

aroyl, heteroaroyl, aryloxy, heteroaryloxy, arylamino or heteroarylamino, which may optionally be substituted with one or more substituents selected from R¹⁸,

or two of R⁵, R⁶, R⁷, R⁸, R⁹, R¹⁰, R¹¹, R¹² and R¹³ in adjacent positions together form a C₁₋₆-alkylene bridge or an —O—C₁₋₆-alkylene-O— bridge,

R¹⁴ and R¹⁵ are independently hydrogen, C₁₋₆-alkyl, aryl-C₁₋₆-alkyl or R¹⁴ and R¹⁵ may together form a C₃₋₆-alkylene bridge

R¹⁶ is independently selected from aryl, heteroaryl, C₃₋₈-cycloalkyl, halogen, trifluoromethyl, trifluoromethoxy, NR¹⁹R²⁰ and C₁₋₆-alkoxy,

R¹⁷ is independently selected from halogen, hydroxy, trifluoromethyl, trifluoromethoxy, C₁₋₆-alkoxy, C₁₋₆-alkyl, amino, C₁₋₆-alkylsulfonyl, C₁₋₆-alkylamino, di-C₁₋₆-alkylamino, cyano, aryl, heteroaryl and C₃₋₈-cycloalkyl,

R¹⁸ is independently selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,

R¹⁹ and R²⁰ are independently hydrogen or C₁₋₆-alkyl, R₁₉ and R₂₀ may together form a C₃₋₆-alkylene bridge,

as well as any diastereomer or enantiomer or tautomeric form thereof including mixtures of these or a pharmaceutically acceptable salt thereof for the preparation of a pharmaceutical composition for the treatment of disorders and diseases related to the histamine H3 receptor.

In another aspect the invention provides the use of a compound of the general formula (II′) as defined above for the preparation of a pharmaceutical composition for the treatment of diseases and disorders in which an inhibition of the H3 histamine receptor has a beneficial effect.

In another aspect the invention provides the use of a compound of the general formula (II′) as defined above for the preparation of a pharmaceutical composition having histamine H3 antagonistic activity or histamine H3 inverse agonistic activity.

In another aspect the invention provides the use of a compound of the general formula (II′) as defined above for the preparation of a pharmaceutical composition for the reduction of weight.

In another aspect the invention provides the use of a compound of the general formula (II′) as defined above for the preparation of a pharmaceutical composition for the treatment of overweight or obesity.

In another aspect the invention provides the use of a compound of the general formula (II′) as defined above for the preparation of a pharmaceutical composition for the suppression of appetite or for satiety induction.

In another aspect the invention provides the use of a compound of the general formula (II′) as defined above for the preparation of a pharmaceutical composition for the prevention and/or treatment of disorders and diseases related to overweight or obesity.

In another aspect the invention provides the use of a compound of the general formula (II′) as defined above for the preparation of a pharmaceutical composition for the prevention and/or treatment of eating disorders such as bulimia and binge eating.

In another aspect the invention provides the use of a compound of the general formula (II′) as defined above for the preparation of a pharmaceutical composition for the treatment of IGT.

In another aspect the invention provides the use of a compound of the general formula (II′) as defined above for the preparation of a pharmaceutical composition for the treatment of type 2 diabetes.

In another aspect the invention provides the use of a compound of the general formula (II′) as defined above for the preparation of a pharmaceutical composition for the delaying or prevention of the progression from IGT to type 2 diabetes.

In another aspect the invention provides the use of a compound of the general formula (II′) as defined above for the preparation of a pharmaceutical composition for the delaying or prevention of the progression from non-insulin requiring type 2 diabetes to insulin requiring type 2 diabetes.

In another aspect the invention provides the use of a compound of the general formula (II′) as defined above for the preparation of a pharmaceutical composition for the treatment of diseases and disorders in which a stimulation of the H3 histamine receptor has a beneficial effect.

In another aspect the invention provides the use of a compound of the general formula (II′) as defined above for the preparation of a pharmaceutical composition having histamine H3 agonistic activity.

In another aspect the invention provides the use of a compound of the general formula (II′) as defined above for the preparation of a pharmaceutical composition for the treatment of allergic rhinitis, ulcer or anorexia.

In another aspect the invention provides the use of a compound of the general formula (II′) as defined above for the preparation of a pharmaceutical composition for the treatment of Alzheimer's disease, narcolepsy or attention deficit disorders.

In another aspect the invention provides a method for the treatment of disorders or diseases related to the H3 histamine receptor the method comprising administering to a subject in need thereof an effective amount of a compound of the general formula (II′) as defined above or a pharmaceutical composition comprising such a compound.

In another aspect the invention provides a method for the treatment of disorders or diseases related to the H3 histamine receptor wherein the effective amount of the compound of the general formula (II′) as defined above is in the range of from about 0.05 mg to about 2000 mg, preferably from about 0.1 mg to about 1000 mg and especially preferred from about 0.5 mg to about 500 mg per day.

In still another aspect, the invention relates to a method for the treatment of diseases and disorders related to the histamine H3 receptor the method comprising administering to a subject in need thereof an effective amount of a compound of the formula (I) or any diastereomer or enantiomer or tautomeric form thereof including mixtures of these or a pharmaceutically acceptable salt thereof or a pharmaceutical composition comprising the same.

In one aspect the invention relates to compounds with histamine H3 receptor antagonistic activity or inverse agonistic activity which may accordingly be useful in the treatment of a wide range of conditions and disorders in which histamine H3 receptor blockade is beneficial. In another aspect the invention relates to compounds with histamine H3 receptor agonistic activity and which may accordingly be useful in the treatment of a wide range of conditions and disorders in which histamine H3 receptor activation is beneficial.

In a preferred embodiment of the invention the present compounds are used for the preparation of a pharmaceutical composition for the reduction of weight.

In a preferred embodiment of the invention the present compounds are used for the preparation of a pharmaceutical composition for the treatment of overweight or obesity.

In another preferred embodiment of the invention the present compounds are used for the preparation of a pharmaceutical composition for the suppression of appetite or satiety induction.

In a further preferred embodiment of the invention the present compounds are used for the preparation of a pharmaceutical composition for the prevention and/or treatment of disorders and diseases related to overweight or obesity such as atherosclerosis, hypertension, IGT (impaired glucose tolerance), diabetes, especially type 2 diabetes (NIDDM (non-insulin dependent diabetes mellitus)), dyslipidaemia, coronary heart disease, gallbladder disease, osteoarthritis and various types of cancer such as endometrial, breast, prostate and colon cancers.

In yet a further preferred embodiment of the invention the present compounds are used for the preparation of a pharmaceutical composition for the prevention and/or treatment of eating disorders such as bulimia and binge eating.

In a further preferred embodiment of the invention the present compounds are used for the preparation of a pharmaceutical composition for the treatment of IGT.

In a further preferred embodiment of the invention the present compounds are used for the preparation of a pharmaceutical composition for the treatment of type 2 diabetes. Such treatment includes inter alia treatment for the purpose of delaying or prevention of the progression from IGT to type 2 diabetes as well as delaying or prevention of the progression from non-insulin requiring type 2 diabetes to insulin requiring type 2 diabetes.

The compounds of the present invention may also be used for the treatment of airway disorders such as asthma, as anti-diarrhoeals and for the modulation of gastric acid secretion. Furthermore, the compounds of the present invention may be used for the treatment of diseases associated with the regulation of sleep and wakefulness and for the treatment of narcolepsy and attention deficit disorders.

Moreover, the compounds of the invention may be used as CNS stimulants or as sedatives. The present compounds may also be used for the treatment of conditions associated with epilepsy. Additionally, the present compounds may be used for the treatment of motion sickness and vertigo. Furthermore, they may be useful as regulators of hypothalamohypophyseal secretion, antidepressants, modulators of cerebral circulation, and in the treatment of irritable bowel syndrome.

Further, the compounds of the present invention may be used for the treatment of dementia and Alzheimer's disease.

The compounds of the present invention may also be useful for the treatment of allergic rhinitis, ulcer or anorexia.

The compounds of the present invention may furthermore be useful for the treatment of migraine, see McLeod et al., The Journal of Pharmacology and Experimental Therapeutics 287 (1998), 43-50, and for the treatment of myocardial infarction, see Mackins et al., Expert Opinion on Investigational Drugs 9 (2000), 2537-2542.

In a further aspect of the invention treatment of a patient with the present compounds is combined with diet and/or exercise.

In a further aspect of the invention the present compounds are administered in combination with one or more further active substances in any suitable ratio(s). Such further active agents may be selected from antiobesity agents, antidiabetics, antidyslipidemic agents, antihypertensive agents, agents for the treatment of complications resulting from or associated with diabetes and agents for the treatment of complications and disorders resulting from or associated with obesity.

Thus, in a further aspect of the invention the present compounds are administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, opioid antagonists (such as naltrexone), exendin-4, GLP-1 and ciliary neurotrophic factor.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

In yet a further aspect the present compounds are administered in combination with one or more antidiabetic agents.

Relevant antidiabetic agents include insulin, insulin analogues and derivatives such as those disclosed in EP 0 792 290 (Novo Nordisk A/S), eg N^{εB29}-tetradecanoyl des (B30) human insulin, EP 0 214 826 and EP 0 705 275 (Novo Nordisk A/S), eg Asp^{B28} human insulin, U.S. Pat. No. 5,504,188 (Eli Lilly), eg Lys^{B28} Pro^{B29} human insulin, EP 0 368 187 (Aventis), eg Lantus®, which are all incorporated herein by reference, GLP-1 derivatives such as those disclosed in WO 98/08871 (Novo Nordisk A/S), which is incorporated herein by reference, as well as orally active hypoglycaemic agents.

The orally active hypoglycaemic agents preferably comprise imidazolines, sulfonylureas, biguanides, meglitinides, oxadiazolidinediones, thiazolidinediones, insulin sensitizers, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S) which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

In one embodiment of the invention the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mix-preparation comprising one or more of these.

In a further embodiment of the invention the present compounds are administered in combination with a sulfonylurea eg tolbutamide, chlorpropamide, tolazamide, glibenclamide, glipizide, glimepiride, glicazide or glyburide.

In another embodiment of the invention the present compounds are administered in combination with a biguanide eg metformin.

In yet another embodiment of the invention the present compounds are administered in combination with a meglitinide eg repaglinide or nateglinide.

In still another embodiment of the invention the present compounds are administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the present compounds may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an α-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In yet another embodiment of the invention the present compounds may be administered in combination with nateglinide.

In still another embodiment, the present compounds are administered in combination with an antihyperlipidemic agent or antilipidemic agent, eg cholestyramine, colestipol, clofibrate, gemfibrozil, lovastatin, pravastatin, simvastatin, probucol or dextrothyroxine.

In still another embodiment of the invention the present compounds are administered in combination with an antilipidemic agent eg cholestyramine, colestipol, clofibrate, gemfibrozil, lovastatin, pravastatin, simvastatin, probucol or dextrothyroxine.

In another aspect of the invention, the present compounds are administered in combination with more than one of the above-mentioned compounds eg in combination with metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

The compounds of the present invention may be chiral, and it is intended that any enantiomers, as separated, pure or partially purified enantiomers or racemic mixtures thereof are included within the scope of the invention.

Furthermore, when a double bond or a fully or partially saturated ring system or more than one center of asymmetry or a bond with restricted rotatability is present in the molecule diastereomers may be formed. It is intended that any diastereomers, as separated, pure or partially purified diastereomers or mixtures thereof are included within the scope of the invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds of the present invention interact with the histamine H3 receptor and are accordingly useful for the treatment of a wide variety of conditions and disorders in which histamine H3 receptor interactions are beneficial.

### Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, preferably from about 0.1 to about 500 mg, and more preferred from about 0.5 mg to about 200 mg. For parenteral routes, such as intravenous, intrathecal, intramuscular and similar administration, typically doses are in the order of about half the dose employed for oral administration. The compounds of this invention are generally utilized as the free substance or as a pharmaceutically acceptable salt thereof. One example is an acid addition salt of a compound having the utility of a free base. When a compound of the formula (I) contains a free base such salts are prepared in a conventional manner by treating a solution or suspension of a free base of the formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain: Core:

| | | |
|---|---|---|
| Active compound (as free compound or salt thereof) | | 5.0 mg |
| Lactosum Ph. Eur. | | 67.8 mg |
| Cellulose, microcryst. (Avicel) | | 31.4 mg |
| Amberlite ® IRP88* | | 1.0 mg |
| Magnesii stearas Ph. Eur. | | q.s. |
| Coating: | | |
| Hydroxypropyl methylcellulose | approx. | 9 mg |
| Mywacett 9-40 T** | approx. | 0.9 mg |

*Polacrillin potassium NF, tablet disintegrant, Rohm and Haas.
**Acylated monoglyceride used as plasticizer for film coating.

If desired, the pharmaceutical composition of the invention may comprise the compound of the formula (I) in combination with further pharmacologically active substances such as those described in the foregoing.

### EXAMPLES

In the examples the following terms are intended to have the following, general meanings:

DIPEA: diisopropylethylamine

DMSO: dimethyl sulphoxide

THF: tetrahydrofuran

HPLC (Method A)

NMR spectra were recorded on Bruker 300 MHz and 400 MHz instruments. HPLC-MS was performed on a Perkin Elmer instrument (API 100). The column used was X-Terra C18, 5 μm, 50×3 mm, and elution was done at 1.5 ml/min at room temperature with a gradient of 5% to 90% acetonitrile in water with 0.01% trifluoroacetic acid within 7.5 min.

HPLC (Method B)

The reverse phase analysis was performed using UV detections at 214 and 254 nm on a 218TP54 4.6 mm×150 mm C-18 silica column, which was eluted at 1 ml/min at 42° C. The column was equilibrated with 5% acetonitrile, 85% water and 10% of a solution of 0.5% trifluoroacetic acid in water and eluted by a linear gradient from 5% acetonitrile, 85% water and 10% of a solution of 0.5% trifluoroacetic acid to 90% acetonitrile and 10% of a solution of 0.5% trifluoroacetic acid over 15 min.

HPLC (Method C)

The RP-analyses was performed using a Alliance Waters 2695 system fitted with a Waters 2487 dualband detector. UV detections were collected using a Symmetry C18 , 3.5 um, 3.0 mm×100 mm column. The eluation is done with a linear gradient of 5-90% acetonitrile, 90-0% water, and 5% trifluoroacetic acid (1.0%) in water over 8 minutes at a flow-rate of 1.0 min/min.

General Procedure (A)

General procedure (A) may be used for the preparation of the compounds of the general formula (Ia):

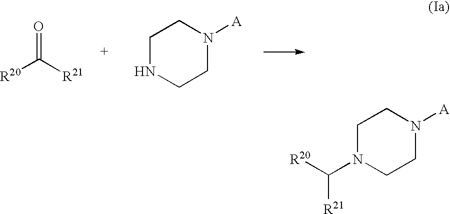

wherein —CH(R²⁰R²¹) represents ethyl, isopropyl, branched C₄₋₆-alkyl, branched C₄₋₆-alkenyl, branched C₄₋₆-alkynyl, C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted with one or more halogen substituents.

To a mixture of a monosubstituted piperazine (15.2 mmol) in a suitable solvent such as and THF are added a ketone or an aldehyde (22.6 mmol), water, acetic acid (45.0 mmol), and then NaCNBH₃ (18 mmol). The mixture is stirred at 55° C. for 5.5 hours (ketones) or at room temperature overnight (aldehydes), and then concentrated under reduced pressure. Saturated aqueous NaHCO₃ solution (100 ml) is added, and the mixture is extracted with a solvent such as ethyl acetate (3×40 ml). The combined extracts are washed with brine, dried over magnesium sulphate, and concentrated under reduced pressure. The residue may be converted into an appropriate salt, such as the hydrochloride salt by co-evaporation with an acid, such as 1 molar aqueous hydrochloric acid, ethanol and toluene, and the residue is then purified by recrystallization.

General Procedure (B)

The compounds of the general formula (I) may be prepared by the general procedure (B):

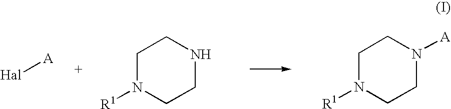

A mixture of a monosubstituted piperazine (2.00 mmol), DMSO (1.0 ml), a suitable aryl or heteroaryl halide (2.00 mmol), and a base such as DIPEA (0.20 ml) is stirred for one hour at 100° C. and then for 18 hours at 120° C. Water and potassium carbonate are added and the mixture is extracted with a solvent such as ethyl acetate (3×20 ml). Isolation and purification are done as in General Procedure (A).

Non-commercially available substituted 2-chloroquinolines were prepared as described in the literature: F. Effenberger, W. Hartmann, Chemische Berichte 1969, 102, 3260-3267.

General Procedure (C)

The compounds of the general formula (I) may be prepared by the general procedure (C):

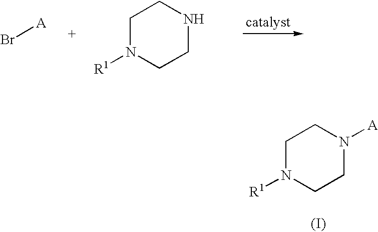

A compound of formula I may be prepared from a suitable monosubstituted piperazine and a suitable aryl bromide in the presence of a suitable catalyst such as e. g. tris(dibenzylideneacetone)dipalladium in a suitable solvent such as toluene at a suitable temperature between 0° C. and 150° C.

### Example 1

### 4-(4-Cyclopentylpiperazin-1-yl)phenol

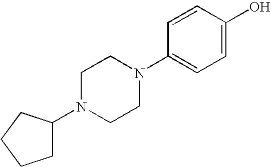

To a suspension of 1-(4-hydroxyphenyl)piperazine (2.70 g, 15.2 mmol) in THF (28 ml) were added cyclopentanone (1.90 ml, 22.6 mmol), water (0.15 ml), acetic acid (2.70 ml, 45.0 mmol), and then NaCNBH₃ (18 ml, 1 molar in THF, 18 mmol). The mixture was stirred at 55° C. for 5.5 hours and then concentrated under reduced pressure. Saturated aqueous Na—HCO₃ solution (100 ml) and ethyl acetate (40 ml) were added, and the mixture was filtered. The resulting solid was resuspended in methanol (30 ml), heated to reflux, and allowed to stand at room temperature over night. Filtration and drying under reduced pressure yielded the title compound (1.82 g, 49%) as a solid.

¹H NMR (DMSO-d₆) δ1.34 (m, 2H), 1.49 (m, 2H), 1.60 (m, 2H), 1.79 (m, 2H), 2.43 (m, 1H), 2.51 (m, 4H), 2.92 (m, 4H), 6.62 (d, J=8 Hz, 2H), 6.77 (d, J=8 Hz, 2H), 8.78 (s, 1H); HPLC-MS: m/z 247 (MH⁺); Rf: 2.70 min.

### Example 2

### 1-Cyclopentyl-4-[4-(4-fluorobenzyloxy)phenyl]piperazine

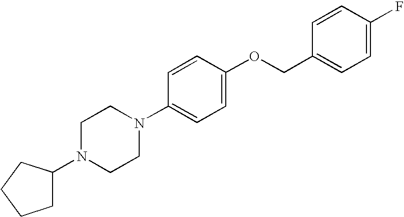

To a suspension of potassium hydroxide (0.165 g, 2.95 mmol) in ethanol (4 ml) was added 4-(4-cyclopentylpiperazin-1-yl)phenol (0.25 g, 1.02 mmol). After 10 min 4-fluorobenzyl chloride (0.18 ml, 0.22 g, 1.51 mmol) was added, and the mixture was stirred at 70° C. for 5 hours. Saturated aqueous NaHCO₃ solution (20 ml) was added, and the mixture was extracted with ethyl acetate (3×20 ml). The combined extracts were washed with brine, dried over magnesium sulphate, and concentrated. Recrystallization from methanol (4 ml) yielded 0.125 g (35%) of the title compound.

¹H NMR (DMSO-d₆) δ1.34 (m, 2H), 1.50 (m, 2H), 1.62 (m, 2H), 1.81 (m, 2H), 2.45 (m, 1H), 2.51 (m, 4H), 2.99 (m, 4H), 5.00 (s, 2H), 6.87 (m, 4H), 7.19 (t, J=8 Hz, 2H), 7.46 (m, 2H); HPLC-MS: m/z 355 (MH⁺); Rf: 4.73 min.

### Example 3

### 1-(3-Chlorophenyl)-4-cyclopentylpiperazine

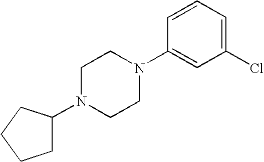

This compound was prepared as described in Example 1, starting from 1-(3-chlorophenyl)-piperazine.

¹H NMR (DMSO-d₆) δ1.34 (m, 2H), 1.50 (m, 2H), 1.60 (m, 2H), 1.80 (m, 2H), 2.45 (m, 1H), 2.51 (m, 4H), 3.14 (m, 4H), 6.78 (d, J=8 Hz, 1H), 6.88 (m, 2H), 7.19 (t, J=8 Hz, 1H); HPLC-MS: m/z 265 (MH⁺); Rf: 3.88 min.

### Example 4

### 1-[4-(4-Cyclopentylpiperazin-1-yl)phenyl]ethanone

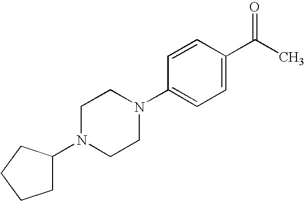

This compound was prepared as described in Example 1, starting from 1-(4-acetylphenyl)-piperazine.

¹H NMR (DMSO-d₆) δ1.30-1.88 (m, 8H), 2.45 (s, 3H), 2.45 (m, 1H), 2.51 (m, 4H), 3.31 (m, 4H), 6.95 (d, J=8 Hz, 2H), 7.79 (d, J=8 Hz, 2H); HPLC-MS: m/z 273 (MH+); Rf: 3.25 min.

### Example 5

### 1-(3,4-Dichlorophenyl)-4-(1-ethylpropyl)piperazine

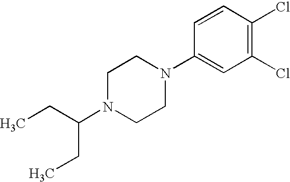

This compound was prepared as described in Example 1, starting from 1-(3,4-dichlorophenyl)piperazine and 3-pentanone.

¹H NMR (DMSO-d₆) δ0.88 (t, J=7 Hz, 6H), 1.28 (m, 2H), 1.45 (m, 2H), 2.19 (m, 1H), 2.56 (br s, 4H), 3.12 (br s, 4H), 6.91 (m, 1H), 7.09 (br s, 1H), 7.36 (d, J=8 Hz, 1H); HPLC-MS: m/z 301 (MH+); Rf: 4.25 min.

### Example 6

### {4-[4-(1-Ethylpropyl)piperazin-1-yl]phenyl}phenylmethanone hydrochloride

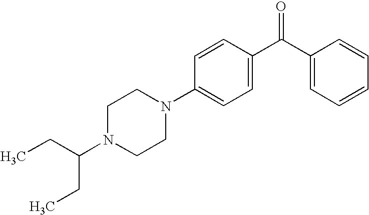

A mixture of 1-(3-pentyl)piperazine (0.31 g, 2.00 mmol), DMSO (1.0 ml), 4-fluorobenzophenone (0.40 g, 2.00 mmol), and DIPEA (0.20 ml) was stirred for one hour at 100° C. and then for 18 hours at 120° C. Water (50 ml) and potassium carbonate (2 g) were added and the mixture was extracted with ethyl acetate (3×20 ml). The combined extracts were washed with brine, dried with magnesium sulphate, and concentrated under reduced pressure. The crude product was redissolved in ethanol (10 ml) and 1 molar aqueous HCl (4 ml), and the solution was concentrated under reduced pressure. After co-evaporation with ethanol and toluene the residue solidified, and was recrystallized from acetonitrile (100 ml). 0.20 g (27%) of the title compound was obtained.

¹H NMR (DMSO-d₆) δ0.95 (m, 6H), 1.62 (m, 2H), 1.89 (m, 2H), 3.04-3.27 (m, 3H), 3.48 (m, 4H), 4.05 (m, 2H), 7.08 (m, 2H), 7.51 (m, 2H), 7.65 (m, 5H), 10.75 (br s, 1H); HPLC-MS: m/z 337 (MH⁺); Rf: 4.27 min.

### Example 7

### 1-(4-Benzylphenyl)-4-(1-ethylpropyl)piperazine hydrochloride

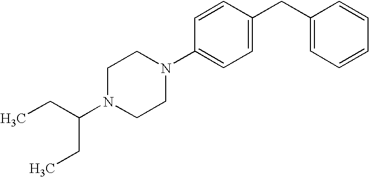

A mixture of {4-[4-(1-ethylpropyl)piperazin-1-yl]phenyl}phenylmethanone hydrochloride (77 mg, 0.21 mmol), trifluoroacetic acid (2.0 ml), and triethylsilane (0.5 ml) was stirred at 60° C. for 20 hours. The mixture was concentrated under reduced pressure, and mixed with water and potassium carbonate. The mixture was extracted with ethyl acetate (3×20 ml). The combined extracts were washed with brine, dried with magnesium sulphate, and concentrated under reduced pressure. The crude product was redissolved in ethanol and 1 molar aqueous HCl, and the solution was concentrated under reduced pressure. After co-evaporation with ethanol and toluene the residue solidified. 45 mg (61%) of the title compound was obtained.

¹H NMR (DMSO-d₆) δ0.98 (t, J=7 Hz, 6H), 1.64 (m, 2H), 1.89 (m, 2H), 3.04-3.23 (m, 5H), 3.48 (m, 2H), 3.72 (m, 2H), 3.85 (s, 2H), 6.93 (d, J=8 Hz, 2H), 7.10-7.30 (m, 7H), 10.05 (br s, 1H); HPLC-MS: m/z 323 (MH⁺); Rf: 4.93 min.

### Example 8

### Cyclopropyl-{4-[4-(1-ethylpropyl)piperazin-1-yl]phenyl}methanone hydrochloride

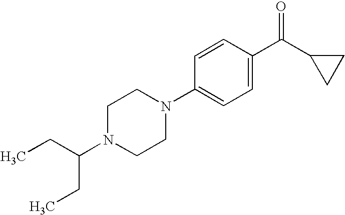

This compound was prepared as described in Example 6, starting from 4-fluorophenyl(cyclopropyl)ketone.

¹H NMR (DMSO-d₆) δ0.98 (m, 10H), 1.64 (m, 2H), 1.89 (m, 2H), 2.82 (br s, 1H), 3.04-3.23 (m, 3H), 3.49 (m, 4H), 4.04 (m, 2H), 7.07 (d, J=8 Hz, 2H), 7.96 (d, J=8 Hz, 2H), 10.95 (br s, 1H); HPLC-MS: m/z 301 (MH⁺); Rf: 4.03 min.

### Example 9

### (2-Chlorophenyl)-{4-[4-(1-ethylpropyl)piperazin-1-yl]phenyl}methanone hydrochloride

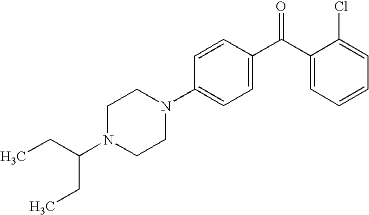

This compound was prepared as described in Example 6, starting from 4-fluorophenyl-(2-chlorophenyl)ketone.

¹H NMR (DMSO-d₆) δ0.98 (t, J=7 Hz, 6H), 1.64 (m, 2H), 1.88 (m, 2H), 3.08-3.23 (m, 3H), 3.50 (m, 4H), 4.06 (m, 2H), 7.08 (d, J=8 Hz, 2H), 7.31 (m, 1H), 7.48 (m, 1H), 7.50-7.61 (m, 4H), 10.85 (br s, 1H); HPLC-MS: m/z 371 (MH⁺); Rf: 4.43 min.

### Example 10

### {4-[4-(1-Ethylpropyl)piperazin-1-yl]phenyl}-(4-fluorophenyl)methanone hydrochloride

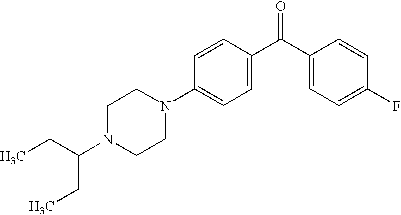

This compound was prepared as described in Example 6, starting from 4,4′-difluorobenzophenone.

¹H NMR (DMSO-d₆) δ0.98 (t, J=7 Hz, 6H), 1.66 (m, 2H), 1.89 (m, 2H), 3.08-3.25 (m, 3H), 3.41-3.53 (m, 4H), 4.05 (m, 2H), 7.09 (d, J=8 Hz, 2H), 7.38 (m, 2H), 7.69 (d, J=8 Hz, 2H), 7.76 (m, 2H), 10.80 (br s, 1H); HPLC-MS: m/z 355 (MH⁺); Rf: 4.37 min.

### Example 11

### 1-Cyclopentyl-4-(6-trifluoromethylpyridin-2-yl)piperazine

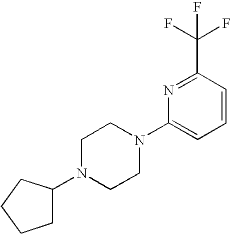

This compound was prepared as described in Example 1, starting from 1-(6-trifluoromethylpyridin-2-yl)piperazine.

¹H NMR (DMSO-d₆) δ1.34 (m, 2H), 1.50 (m, 2H), 1.60 (m, 2H), 1.80 (m, 2H), 2.45-2.51 (m, 5H), 3.52 (m, 4H), 7.02 (d, J=8 Hz, 1H), 7.11 (d, J=8 Hz, 1H), 7.71 (t, J=8 Hz, 1H); HPLC-MS: m/z 300 (MH⁺); Rf: 4.10 min.

### Example 12

### 1-Cyclopentyl-4-(5-trifluoromethylpyridin-2-yl)piperazine

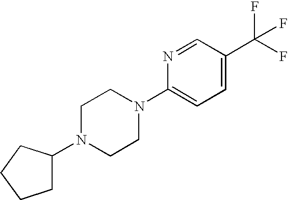

This compound was prepared as described in Example 1, starting from 1-(5-trifluoromethylpyridin-2-yl)piperazine.

¹H NMR (DMSO-d₆) δ1.36 (m, 2H), 1.50 (m, 2H), 1.60 (m, 2H), 1.80 (m, 2H), 2.45-2.52 (m, 5H), 3.58 (m, 4H), 6.92 (d, J=8 Hz, 1H), 7.78 (br d, J=8 Hz, 1H), 8.39 (s, 1H); HPLC-MS: m/z 300 (MH⁺); Rf: 3.87 min.

### Example 13

### 1-Cyclopentyl-4-(3-trifluoromethylpyridin-2-yl)piperazine

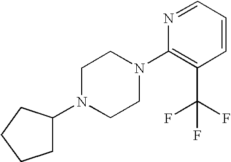

This compound was prepared as described in Example 1, starting from 1-(3-trifluoromethylpyridin-2-yl)piperazine.

¹H NMR (DMSO-d₆) δ1.29-1.65 (m, 6H), 1.80 (m, 2H), 2.45 (m, 1H), 2.52 (m, 4H), 3.18 (m, 4H), 7.16 (m, 1H), 8.02 (m, 1H), 8.49 (m, 1H); HPLC-MS: m/z 300 (MH⁺); Rf: 3.70 min.

### Example 14

### 2-[4-(1-Ethylpropyl)piperazin-1-yl]quinoline hydrochloride

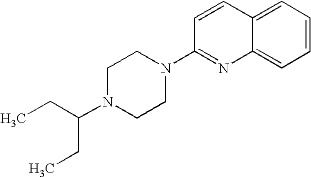

This compound was prepared as described in Example 6, starting from 2-chloroquinoline.

1H NMR (DMSO-d6) δ0.99 (t, J=7 Hz, 6H), 1.65 (m, 2H), 1.94 (m, 2H), 3.12 (br s, 1H), 3.33 (m, 2H), 3.57 (m, 2H), 3.93 (m, 2H), 4.83 (m, 2H), 7.44-7.58 (m, 2H), 7.76 (m, 1H), 7.92 (m, 1H), 8.25 (br s, 1H), 8.42 (m, 1H), 11.20 (br s, H); HPLC-MS: m/z 284 (MH⁺); Rf: 3.03 min

### Example 15

### 7-Chloro-4-[4-(1-ethylpropyl)piperazin-1-yl]quinoline hydrochloride

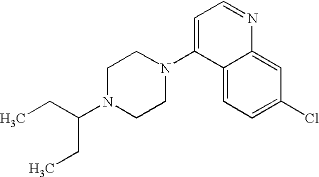

This compound was prepared as described in Example 6, starting from 4,7-dichloroquinoline.

¹H NMR (DMSO-d₆) δ1.00 (t, J=7 Hz, 6H), 1.67 (m, 2H), 1.95 (m, 2H), 3.15 (br s, 1H), 3.30-3.70 (m, 4H), 4.05 (m, 2H), 4.20 (m, 2H), 7.32 (m, 1H), 7.73 (m, 1H), 8.28 (m, 2H), 8.83 (m, 1H), 11.35 (br s, H); HPLC-MS: m/z 318 (MH⁺); Rf: 3.13 min.

### Example 16

### [4-(4-Cyclopentylpiperazin-1-yl)phenyl]-(3,4-dimethoxyphenyl)methanone hydrochloride

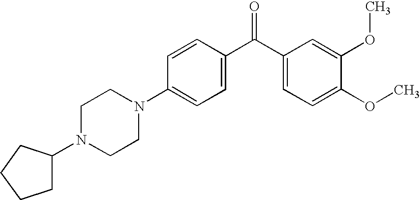

This compound was prepared as described in Example 6, starting from 4′-fluoro-3,4-dimethoxybenzophenone.

¹H NMR (DMSO-d₆) δ1.55 (m, 2H), 1.65-1.90 (m, 4H), 2.02 (m, 2H), 3.05-3.40 (m, 4H), 3.55 (m, 3H), 3.81 (s, 3H), 3.88 (s, 3H), 4.08 (m, 2H), 7.10 (m, 3H), 7.29 (m, 2H), 7.69 (d, J=8 Hz, 2H), 10.78 (br s, 1H); HPLC-MS: m/z 395 (MH⁺); Rf: 3.03 min.

### Example 17

### [4-(4-Cyclopentylpiperazin-1-yl)-3,5-difluorophenyl]phenylmethanone hydrochloride

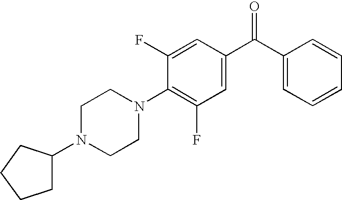

This compound was prepared as described in Example 6, starting from 3,4,5-trifluorobenzophenone.

¹H NMR (DMSO-d₆) δ1.55 (m, 2H), 1.65-1.90 (m, 4H), 2.02 (m, 2H), 3.15 (m, 2H), 3.50-3.71 (m, 7H), 7.42 (m, 2H), 7.58 (m, 2H), 7.68-7.78 (m, 3H), 10.90 (br s, 1H); HPLC-MS: m/z 371 (MH⁺); Rf: 2.77 min.

### Example 18

### 2-(4-Cyclopentylpiperazin-1-yl)quinoxaline hydrochloride

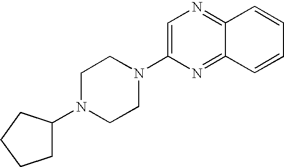

This compound was prepared as Example 6, starting from 2-chloroquinoxaline and using propionitrile as solvent.

¹H NMR (DMSO-d₆) δ1.55 (m, 2H), 1.64-1.90 (m, 4H), 2.02 (m, 2H), 3.15 (m, 2H), 3.42-3.65 (m, 5H), 4.71 (m, 2H), 7.49 (m, 1H), 7.67 (m, 2H), 7.88 (br d, J=8 Hz, 1H), 8.91 (s, 1H), 10.92 (br s, 1H); HPLC-MS: m/z 283 (MH⁺); Rf: 1.70 min.

### Example 19

### 2-(4-Cyclopropylmethylpiperazin-1-yl)quinoxaline hydrochloride

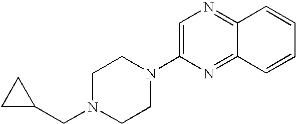

¹H NMR (DMSO-d₆) δ0.41 (m, 2H), 0.66 (m, 2H), 1.18 (m, 1H), 3.02 (m, 2H), 3.13 (m, 2H), 3.52-3.69 (m, 4H), 4.71 (m, 2H), 7.48 (m, 1H), 7.66 (m; 2H), 7.88 (d, J=8 Hz, 1H), 8.90 (s, 1H), 11.17 (br s, 1H); HPLC-MS: m/z 269 (MH⁺); Rf: 1.73 min.

### Example 20

### [6-(4-Cyclopentylpiperazin-1-yl)pyridin-3-yl]piperidin-1-ylmethanone hydrochloride

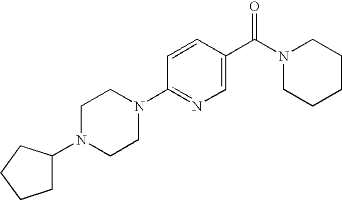

¹H NMR (DMSO-d₆) δ1.45-2.08 (m, 14H), 3.06 (m, 2H), 3.38-3.61 (m, 9H), 4.44 (m, 2H), 7.02 (d, J=8 Hz, 1H), 7.70 (dd, J=8 Hz, 1 Hz, 1H), 8.19 (d, J=1 Hz, 1H); HPLC-MS: m/z (MH⁺).

This compound was prepared as Example 6 (General Procedure B), using 1-(6-chloronicotinoyl)piperidine (Thunus, Ann. Pharm. Fr. 1977, 35, 197).

### Example 21

### 2-(4-Cyclopentylpiperazin-1-yl)quinoline hydrochloride

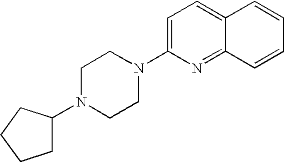

¹H NMR (DMSO-d₆) δ1.62 (m, 2H), 1.82 (m, 2H), 1.96 (m, 2H), 2.09 (m, 2H), 3.25 (m, 2H), 3.55-3.70 (m, 5H), 4.83 (m, 2H), 7.46-7.60 (m, 2H), 7.80 (m, 1H), 7.94 (m, 1H), 8.13 (m, 1H), 8.42 (m, 1H), 11.52 (br s, 1H); HPLC-MS: m/z 282 (MH⁺); Rf: 0.34 min.

This compound was prepared as Example 6, starting from 2-chloroquinoline.

### Example 22

### 2-(4-Cyclopentylpiperazin-1-yl)-7-methoxy-3-(4-methoxyphenyl)quinoline hydrochloride

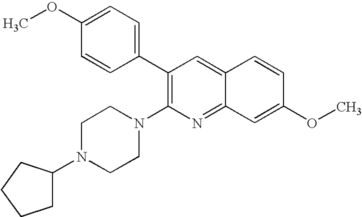

¹H NMR (DMSO-d₆) δ1.53 (m, 2H), 1.63-1.86 (m, 4H), 1.98 (m, 2H), 3.05 (m, 2H), 3.33-3.52 (m, 5H), 3.75 (m, 2H), 3.82 (s, 3H), 3.91 (s, 3H), 7.08 (d, J=8 Hz, 2H), 7.13 (dd, J=8 Hz, 1 Hz, 1H), 7.49 (br s, 1H), 7.61 (d, J=8 Hz, 2H), 7.83 (d, J=8 Hz, 1H), 8.15 (s, 1H), 11.29 (br s, 1H); HPLC-MS: m/z 418 (MH⁺); Rf: 3.40 min.

### Example 23

### {6-[4-(1-Cyclopropyl-1-methylethyl)piperazin-1-yl]pyridin-3-yl}phenylmethanone hydrochloride

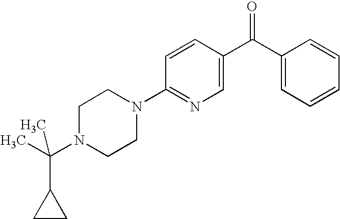

¹H NMR (DMSO-d₆) δ0.48-0.62 (m, 4H), 1.22-1.39 (m, 7H), 3.14 (m, 2H), 3.69 (m, 4H), 4.64 (m, 2H), 7.08 (d, J=8 Hz, 1H), 7.55 (m, 2H), 7.61-7.72 (m, 3H), 8.00 (dd, J=8 Hz, 1 Hz, 1H), 8.52 (d, J=1 Hz, 1H), 11.27 (br s, 1H); HPLC-MS: m/z 350 (MH⁺); Rf: 3.03 min.

This compound was prepared as Example 6, starting from 2-chloro-5-benzoylpyridine (T. D. Penning et al., J. Med. Chem. 2000, 43, 721-735).

### Example 24

### {4-[4-(1-Cyclopropyl-1-methylethyl)piperazin-1-yl]-3,5-difluorophenyl}phenylmethanone hydrochloride

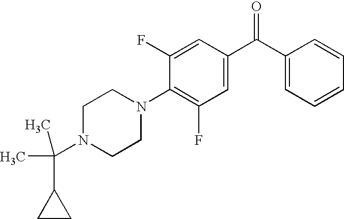

¹H NMR (DMSO-d₆) δ0.48-0.63 (m, 4H), 1.23-1.40 (m, 7H), 3.18 (m, 2H), 3.56 (m, 2H), 3.69 (m, 2H), 3.84 (m, 2H), 7.42 (m, 2H), 7.58 (m, 2H), 7.66-7.76 (m, 3H), 10.90 (br s, 1H); HPLC-MS: m/z 385 (MH⁺); Rf: 3.73 min.

### Example 25

### {4-[4-(1-Cyclopropyl-1-methylethyl)piperazin-1-yl]-3,5-difluorophenyl}phenylmethanol hydrochloride

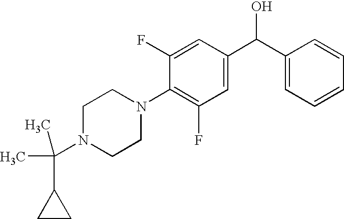

¹H NMR (DMSO-d₆) δ0.47-0.61 (m, 4H), 1.23-1.34 (m, 7H), 3.12 (m, 2H), 3.29 (m, 2H), 3.65 (m, 4H), 5.66 (m, 1H), 6.08 (m, 1H), 7.06 (m, 2H), 7.19-7.40 (m, 5H), 10.40 (br s, 1H); HPLC-MS: m/z 387 (MH⁺); Rf: 3.40 min.

This compound was prepared by reduction with sodium borohydride of Example 24.

### Example 26

### [4-(4-Cyclopropylmethylpiperazin-1-yl)-3,5-difluorophenyl]-(4-fluorophenyl)methanone hydrochloride

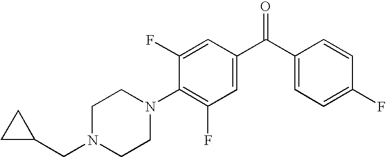

¹H NMR (DMSO-d₆) δ0.40 (m, 2H), 0.66 (m, 2H), 1.13 (m, 1H), 3.02-3.23 (m, 5H), 3.52-3.68 (m, 5H), 7.40 (m, 4H), 7.82 (m, 2H), 10.55 (br s, 1H); HPLC-MS: m/z 375 (MH⁺); Rf: 2.78 min.

This compound was prepared as example 6, using 3,4,5,4′-tetrafluorobenzophenone. The latter was prepared by Friedel Crafts acylation of fluorobenzene with 3,4,5-trifluorobenzoyl chloride.

### Example 27

### {4-[4-(1-Ethylpropyl)piperazin-1-yl]-3,5-difluorophenyl}-(4-fluorophenyl)methanone hydrochloride

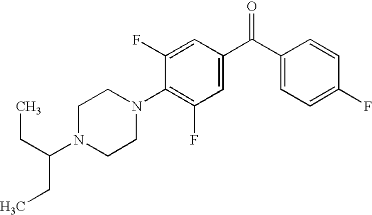

¹H NMR (DMSO-d₆) δ0.97 (t, J=7 Hz, 6H), 1.68 (m, 2H), 1.88 (m, 2H), 3.06-3.26 (m, 3H), 3.52 (m, 4H), 3.75 (m, 2H), 7.41 (m, 4H), 7.82 (m, 2H), 10.31 (br s, 1H); HPLC-MS: m/z 391 (MH⁺); Rf: 3.00 min.

### Example 28

### 2-[4-(1-Ethylpropyl)piperazin-1-yl]-6,7-dimethoxyquinoline hydrochloride

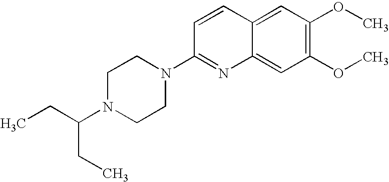

¹H NMR (DMSO-d₆) δ0.99 (t, J=7 Hz, 6H), 1.66 (m, 2H), 1.91 (m, 2H), 3.11 (m, 1H), 3.29 (m, 2H), 3.56 (m, 4H), 3.88 (s, 3H), 3.92 (s, 3H), 4.72 (m, 2H), 7.31-7.43 (m, 2H), 7.28 (br s, 1H), 8.30 (br s, 1H), 10.95 (br s, 1H); HPLC-MS: m/z 344 (MH⁺); Rf: 2.00 min.

This compound was prepared as example 6 from 2-chloro-6,7-dimethoxyquinoline (Pettit, Can. J. Chem. 1964, 42, 1764).

### Example 29

### 2-[4-(1-Ethylpropyl)piperazin-1-yl]-4-trifluoromethylquinoline hydrochloride

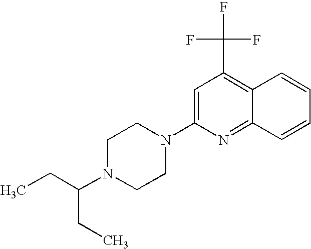

¹H NMR (DMSO-d₆) δ0.99 (t, J=7 Hz, 6H), 1.65 (m, 2H), 1.91 (m, 2H), 3.08 (m, 1H), 3.19 (m, 2H), 3.52 (m, 2H), 3.75 (m, 2H), 4.77 (m, 2H), 7.48 (t, J=7 Hz, 1H), 7.72 (m, 2H), 7.88 (m, 2H), 11.19 (br s, 1H); HPLC-MS: m/z 352 (MH⁺); Rf: 3.70 min.

This compound was prepared from 2-chloro-4-trifluoromethylquinoline. The latter was prepared as described in the literature: R. D. Westland et al. J. Med. Chem. 1973, 16, 319-327.

### Example 30

### 2-(4-Cyclopropylmethylpiperazin-1-yl)-6-methoxy-4-trifluoromethylquinoline hydrochloride

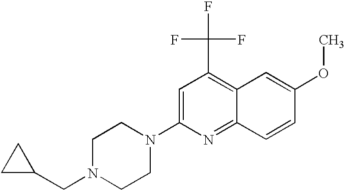

¹H NMR (DMSO-d₆) δ0.40 (m, 2H), 0.67 (m, 2H), 1.14 (m, 1H), 3.01-3.16 (m, 4H), 3.46 (m, 2H), 3.64 (m, 2H), 3.87 (s, 3H), 4.66 (m, 2H), 7.16 (br s, 1H), 7.43 (dd, J=7 Hz, 1 Hz, 1H), 7.68 (s, 1H), 7.73 (d, J=7 Hz, 1H), 10.70 (br s, 1H); HPLC-MS: m/z 366 (MH⁺); Rf: 3.63 min.

### Example 31

### [4-(4-Cyclopropylmethylpiperazin-1-yl)-3,5-difluorophenyl]phenylmethanone hydrochloride

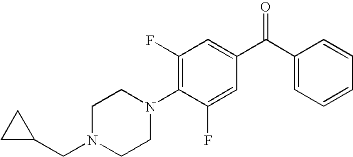

¹H NMR (DMSO-d₆) δ0.40 (m, 2H), 0.67 (m, 2H), 1.14 (m, 1H), 3.03-3.20 (m, 4H), 3.60 (m, 6H), 7.40 (m, 2H), 7.58 (m, 2H), 7.70 (m, 3H), 10.60 (br s, 1H); HPLC-MS: m/z 357 (MH⁺); Rf: 3.53 min.

### Example 32

### [4-(4-Cyclopropylmethylpiperazin-1-yl)-3,5-difluorophenyl]-(3-fluoro-4-methoxyphenyl)methanone hydrochloride

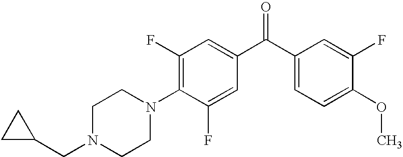

¹H NMR (DMSO-d₆) δ0.41 (m, 2H), 0.65 (m, 2H), 1.15 (m, 1H), 3.06 (m, 2H), 3.18 (m, 2H), 3.50-3.70 (m, 6H), 3.95 (s, 3H), 7.33 (t, J=8 Hz, 1H), 7.41 (m, 2H), 7.60 (m, 2H), 10.79 (br s, 1H); HPLC-MS: m/z 405 (MH⁺); Rf: 3.67 min.

### Example 33

### {6-[4-(1-Ethylpropyl)piperazin-1-yl]pyridin-3-yl}phenylmethanone hydrochloride

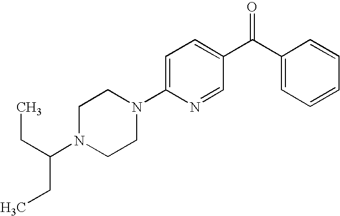

¹H NMR (DMSO-d₆) δ0.97 (t, J=7 Hz, 6H), 1.65 (m, 2H), 1.90 (m, 2H), 3.02-3.22 (m, 3H), 3.49-3.69 (m, 4H), 4.60 (m, 2H), 7.08 (d, J=8 Hz, 1H), 7.56 (m, 2H), 7.68 (m, 3H), 7.99 (dd, J=8 Hz, 1 Hz, 1H), 8.50 (d, J=1 Hz, 1H), 10.90 (br s, 1H); HPLC-MS: m/z 338 (MH⁺); Rf: 3.00 min.

### Example 34

### {2-[4-(1-Ethylpropyl)piperazin-1-yl]pyridin-4-yl}phenylmethanone hydrochloride

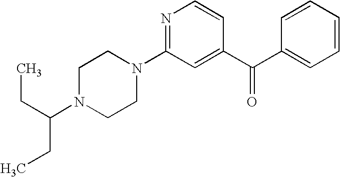

¹H NMR (DMSO-d₆) δ0.97 (t, J=7 Hz, 6H), 1.63 (m, 2H), 1.85 (m, 2H), 3.13 (m, 3H), 3.47 (m, 4H), 4.43 (m, 2H), 6.91 (d, J=6 Hz, 1H), 7.14 (s, 1H), 7.58 (t, J=8 Hz, 2H), 7.70-7.84 (m, 3H), 8.33 (d, J=6 Hz, 1H), 10.43 (br s, 1H); HPLC-MS: m/z 338 (MH⁺); Rf: 2.97 min.

This compound was prepared from 2-chloro-4-benzoylpyridine, which was prepared by Friedel-Crafts acylation of benzene with 2-chloro-4-chlorocarbonylpyridine.

### Example 35

### {4-[4-(1-Ethylpropyl)piperazin-1-yl]phenyl}-(4-hydroxyphenyl)methanone hydrochloride

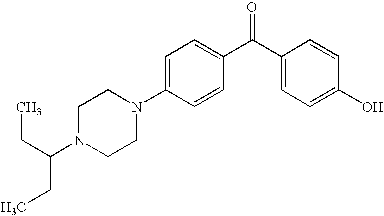

¹H NMR (DMSO-d₆) δ0.97 (t, J=7 Hz, 6H), 1.65 (m, 2H), 1.92 (m, 2H), 3.05-3.25 (m, 3H), 3.35-3.55 (m, 4H), 4.02 (m, 2H), 6.89 (d, J=8 Hz, 2H), 7.08 (d, J=8 Hz, 2H), 7.59 (d, J=8 Hz, 2H), 7.63 (d, J=8 Hz, 2H), 10.36 (s, 1H), 10.60 (br s, 1H); HPLC-MS: m/z 353 (MH⁺); Rf: 2.13 min.

### Example 36

### {6-[4-(1-Ethylpropyl)piperazin-1-yl]pyridin-3-yl}piperidin-1-yl-methanone hydrochloride

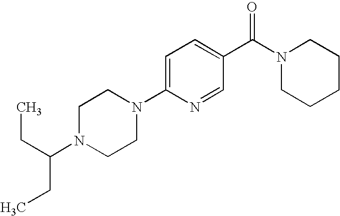

¹H NMR (DMSO-d₆) δ0.97 (t, J=7 Hz, 6H), 1.50 (m, 4H), 1.63 (m, 4H), 1.89 (m, 2H), 3.05-3.20 (m, 3H), 3.50 (m, 8H), 4.46 (m, 2H), 7.04 (m, 1H), 7.70 (m, 1H), 8.18 (br s, 1H), 10.90 (br s, 1H); HPLC-MS: m/z 345 (MH⁺); Rf: 2.27 min.

### Example 37

### N-Benzyl-6-[4-(1-ethylpropyl)piperazin-1-yl]-N-methylnicotinamide hydrochloride

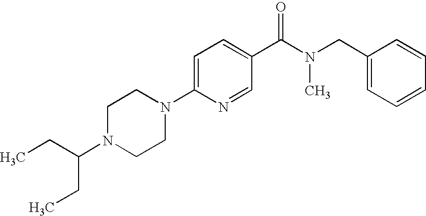

¹H NMR (DMSO-d₆) δ0.97 (t, J=7 Hz, 6H), 1.63 (m, 2H), 1.88 (m, 2H), 2.89 (s, 3H), 3.09 (m, 3H), 3.50 (m, 4H), 4.45 (m, 2H), 4.62 (br s, 2H), 7.02 (d, J=8 Hz, 1H), 7.25-7.41 (m, 5H), 7.78 (m, 1H), 8.28 (br s, 1H), 10.78 (br s, 1H); HPLC-MS: m/z 381 (MH⁺); Rf: 3.10 min.

### Example 38

### 2-[4-(1-Ethylpropyl)piperazin-1-yl]-6-methoxyquinoline hydrochloride

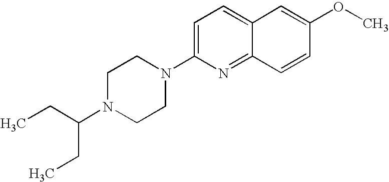

¹H NMR (DMSO-d₆) δ0.98 (t, J=7 Hz, 6H), 1.66 (m, 2H), 1.92 (m, 2H), 3.11 (m, 1H), 3.31 (m, 2H), 3.57 (m, 2H), 3.82 (m, 2H), 3.88 (s, 3H), 4.74 (m, 2H), 7.41 (br s, 2H), 7.53 (m, 1H), 8.12 (br s, 1H), 8.34 (br s, 1H), 10.95 (br s, 1H); HPLC-MS: m/z 314(MH⁺); Rf: 2.17 min.

### Example 39

### 6-[4-(1-Ethylpropyl)piperazin-1-yl]-N-methyl-N-phenylnicotinamide hydrochloride

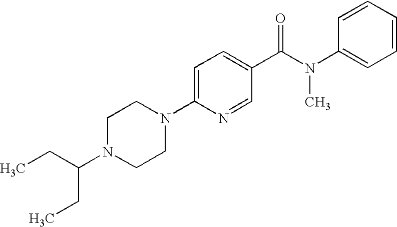

¹H NMR (DMSO-d₆) δ0.95 (t, J=7 Hz, 6H), 1.62 (m, 2H), 1.83 (m, 2H), 3.05 (m, 3H), 3.34 (s, 3H), 3.43 (m, 4H), 4.35 (m, 2H), 6.76 (d, J=8 Hz, 1H), 7.21 (m, 3H), 7.31 (m, 2H), 7.42 (dd, J=8 Hz, 1 Hz, 1H), 8.01 (d, J=1 Hz, 1H), 10.54 (br s, 1H); HPLC-MS: m/z 367 (MH⁺); Rf: 2.90 min.

### Example 40

### {6-[4-(1-Ethylpropyl)piperazin-1-yl]pyridin-3-yl}-(4-fluorophenyl)methanone hydrochloride

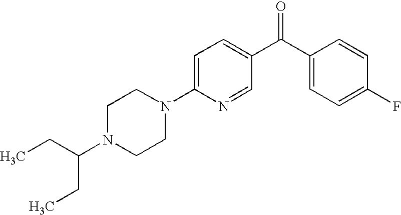

¹H NMR (DMSO-d₆) δ0.95 (t, J=7 Hz, 6H), 1.62 (m, 2H), 1.83 (m, 2H), 3.10 (m, 3H), 3.45-3.65 (m, 4H), 4.55 (m, 2H), 7.05 (d, J=8 Hz, 1H), 7.38 (d, J=8 Hz, 2H), 7.78 (dd, J=8 Hz, 4 Hz, 2H), 7.96 (dd, J=8 Hz, 1 Hz, 1H), 8.48 (d, J=1 Hz, 1H), 10.85 (br s, 1H); HPLC-MS: m/z 356 (MH⁺); Rf: 2.40 min.

### Example 41

### 2-[4-(1-Ethylpropyl)piperazin-1-yl]-4-methylquinoline hydrochloride

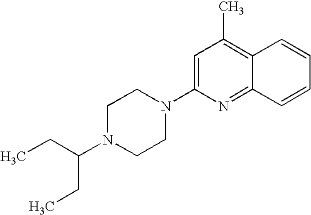

¹H NMR (DMSO-d₆) δ0.98 (t, J=7 Hz, 6H), 1.64 (m, 2H), 1.92 (m, 2H), 2.69 (s, 3H), 3.12 (m, 1H), 3.32 (m, 2H), 3.57 (m, 2H), 3.94 (m, 2H), 4.86 (m, 2H), 7.53 (br s, 2H), 7.80 (m, 1H), 8.01 (m, 1H), 8.32 (br s, 1H), 11.20 (br s, 1H); HPLC-MS: m/z 298 (MH⁺); Rf: 1.26 min.

### Example 42

### 2-[4-(1-Ethylpropyl)piperazin-1-yl]-5,6,7,8-tetrahydroquinoline hydrochloride

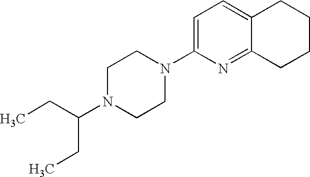

¹H NMR (DMSO-d₆) δ0.98 (t, J=7 Hz, 6H), 1.50-2.03 (m, 8H), 2.63 (m, 2H), 2.90 (m, 2H), 3.00-3.33 (m, 3H), 3.50 (m, 2H), 3.75 (m, 2H), 4.48 (m, 2H), 7.13 (br s, 1H), 7.75 (br s, 1H), 11.10 (br s, 1H); HPLC-MS: m/z 288 (MH⁺); Rf: 1.83 min.

This compound was prepared from 2-chloro-5,6,7,8-tetrahydroquinoline (S. C. Zimmerman, Z. Zeng, J. Org. Chem. 1990, 55, 4789-5791).

### Example 43

### 2-(4-Cyclopropylmethylpiperazin-1-yl)-6-methoxyquinoline hydrochloride

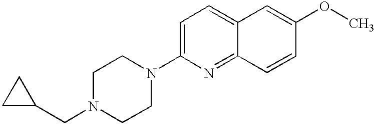

¹H NMR (DMSO-d₆) δ0.41 (m, 2H), 0.66 (m, 2H), 1.18 (m, 1H), 3.02 (m, 2H), 3.13 (m, 2H), 3.52-3.69 (m, 4H), 3.85 (s, 3H), 4.71 (m, 2H), 7.41 (br s, 2H), 7.53 (m, 1H), 8.12 (br s, 1H), 8.34 (br s, 1H), 11.38 (br s, 1H); HPLC-MS: m/z 298 (MH⁺); Rf: 1.87 min.

### Example 44

### 2-(4-Isopropylpiperazin-1-yl)-6-methoxyquinoline hydrochloride

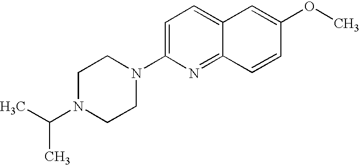

¹H NMR (DMSO-d₆) δ1.32 (d, J=7 Hz, 6H), 3.28 (m, 2H), 3.53 (m, 3H), 3.80 (m, 1H), 3.85 (s, 3H), 4.85 (m, 2H), 7.43 (br s, 2H), 7.59 (d, J=8 Hz, 1H), 8.27 (br s, 1H), 8.34 (br s, 1H), 8.40 (d, J=8 Hz, 1H), 11.60 (br s, 1H); HPLC-MS: m/z 286 (MH⁺); Rf: 1.77 min.

### Example 45

### 2-[4-(1-Ethylpropyl)piperazin-1-yl]-6-fluoro-4-methylquinoline hydrochloride

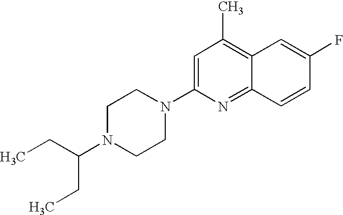

¹H NMR (DMSO-d6) δ0.98 (t, J=7 Hz, 6H), 1.64 (m, 2H), 1.92 (m, 2H), 2.67 (s, 3H), 3.12 (m, 1H), 3.32 (m, 2H), 3.57-4.00 (m, 4H), 4.85 (m, 2H), 7.57 (br s, 2H), 7.68 (m, 1H), 7.82 (m, 1H), 8.33 (br s, 1H), 11.10 (br s, 1H); HPLC-MS: m/z 316 (MH⁺); Rf: 1.92 min.

This compound was prepared from 2-chloro-6-fluoro-4-methylquinoline, which was prepared by acetoacetylation of 4-fluoroaniline, followed by acid-mediated ring-closure and conversion of the resulting carbostyryl into the chloroquinoline by treatment with phosphorus oxychloride.

### Example 46

### 2-(4-Isopropylpiperazin-1-yl)quinoline hydrochloride

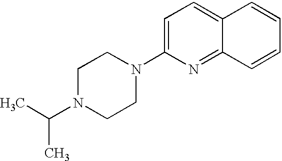

¹H NMR (DMSO-d₆) δ1.31 (d, J=7 Hz, 6H), 3.19 (m, 2H), 3.52 (m, 3H), 3.72 (m, 2H), 4.79 (m, 2H), 7.45 (m, 2H), 7.68 (m, 1H), 7.86 (m, 1H), 8.03 (m, 1H), 8.31 (m, 1H), 11.45 (br s, 1H); HPLC-MS: m/z 256 (MH⁺); Rf: 1.47 min.

### Example 47

### 2-(4-Cyclopropylpiperazin-1-yl)-6-methoxyquinoline hydrochloride

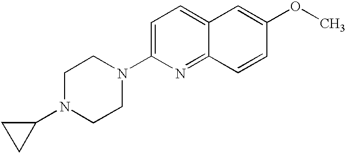

¹H NMR (DMSO-d₆) 50.81 (m, 2H), 1.20 (br s, 2H), 2.86 (br s, 1H), 3.25-3.75 (m, 4H), 3.85 (s, 3H), 4.09 (m, 2H), 4.73 (m, 2H), 7.41 (m, 2H), 7.55 (m, 1H), 8.14 (m, 1H), 8.37 (m, 1H), 11.51 (br s, 1H); HPLC-MS: m/z 284 (MH⁺); Rf: 1.80 min.

### Example 48

### 2-(4-Isopropyl-piperazin-1-yl)-6-trifluoromethoxyquinoline hydrochloride

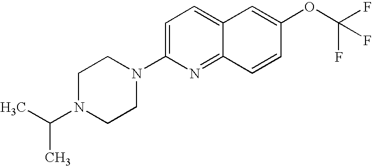

¹H NMR (DMSO-d₆) δ1.31 (d, J=7 Hz, 6H), 3.17 (m, 2H), 3.52 (m, 3H), 3.68 (m, 2H), 4.79 (m, 2H), 7.56 (d, J=8 Hz, 1H), 7.68 (br d, J=7 Hz, 1H), 7.91 (br s, 1H), 8.04 (br s, 1H), 8.35 (br d, J=7 Hz, 1H), 11.28 (br s, 1H); HPLC-MS: m/z 340 (MH⁺); Rf: 3.04 min.

### Example 49

### 6-Chloro-2-(4-cyclopropyl-piperazin-1-yl)-quinoline hydrochloride

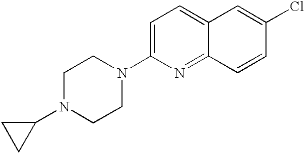

¹H NMR (DMSO-d₆) δ0.81 (m, 2H), 1.14 (br s, 2H), 2.88 (br s, 1H), 3.25-3.70 (m, 6H), 4.67 (m, 2H), 7.44 (d, J=8 Hz, 1H), 7.61 (d, J=8 Hz, 1H), 7.72 (m, 1H), 7.91 (br s, 1H), 8.18 (br d, J=8 Hz, 1H), 10.75 (br s, 1H); HPLC-MS: m/z 288 (MH⁺); Rf: 1.77 min.

### Example 50

### 2-(4-Cyclopropyl-piperazin-1-yl)-6-trifluoromethoxyquinoline hydrochloride

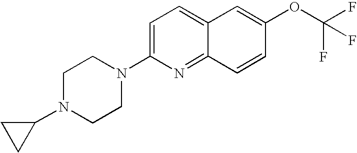

¹H NMR (DMSO-d₆) δ0.81 (m, 2H), 1.15 (br s, 2H), 2.88 (br s, 1H), 3.20-3.70 (m, 6H), 4.68 (m, 2H), 7.49 (d, J=8 Hz, 1H), 7.59 (br d, J=8 Hz, 1H), 7.82 (m, 2H), 8.27 (d, J=8 Hz, 1H), 10.89 (br s, 1H); HPLC-MS: m/z 338 (MH⁺); Rf: 2.24 min.

### Example 51

### 2-(4-Isopropyl-piperazin-1-yl)-8-trifluoromethylquinoline hydrochloride

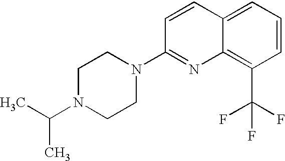

¹H NMR (DMSO-d₆) δ1.31 (d, J=7 Hz, 6H), 3.10 (m, 2H), 3.51 (m, 5H), 4.72 (m, 2H), 7.38 (t, J=8 Hz, 1H), 7.46 (d, J=8 Hz, 1H), 7.95 (br d, J=7 Hz, 1H), 8.05 (br d, J=7 Hz, 1H), 8.26 (d, J=8 Hz, 1H), 10.66 (br s, 1H); HPLC-MS: m/z 324 (MH⁺); Rf: 3.08 min.

### Example 52

### 2-(4-Isopropyl-piperazin-1-yl)-6-trifluoromethylquinoline hydrochloride

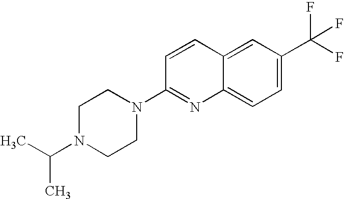

¹H NMR (DMSO-d₆) δ1.31 (d, J=7 Hz, 6H), 3.12 (m, 2H), 3.50-3.68 (m, 5H), 4.78 (m, 2H), 7.52 (d, J=7 Hz, 1H), 7.88 (m, 2H), 8.26 (br s, 1H), 8.36 (d, J=7 Hz, 1H), 10.95 (br s, 1H); HPLC-MS: m/z 324 (MH⁺); Rf: 2.11 min.

### Example 53

### 2-(4-Isopropyl-piperazin-1-yl)-6-propylquinoline hydrochloride

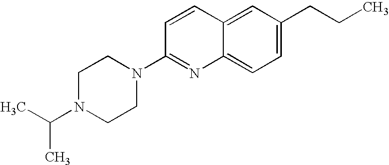

¹H NMR (DMSO-d₆) δ0.91 (t, J=7 Hz, 3H), 1.31 (d, J=7 Hz, 6H), 1.66 (sext, J=7 Hz, 2H), 2.70 (t, J=7 Hz, 2H), 3.23 (m, 2H), 3.48-3.90 (m, 5H), 4.81 (m, 2H), 7.52 (m, 1H), 7.63 (m, 1H), 7.70 (br s, 1H), 8.09 (br s, 1H), 8.34 (br s, 1H), 11.35 (br s, 1H); HPLC-MS: m/z 298 (MH⁺); Rf: 1.97 min.

### Example 54

### 6,8-Difluoro-2-(4-isopropylpiperazin-1-yl)quinoline hydrochloride

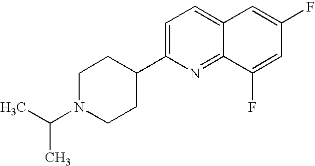

¹H NMR (DMSO-d₆) δ1.31 (d, J=7 Hz, 6H), 3.09 (m, 2H), 3.51 (m, 5H), 4.66 (m, 2H), 7.45-7.58 (m, 3H), 8.18 (d, J=7 Hz, 1H), 10.92 (br s, 1H).

### Example 55

### 8-Fluoro-2-(4-isopropylpiperazin-1-yl)quinoline hydrochloride

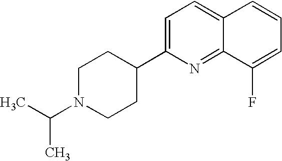

¹H NMR (DMSO-d₆) δ1.31 (d, J=7 Hz, 6H), 3.09 (m, 2H), 3.52 (m, 5H), 4.70 (m, 2H), 7.24 (m, 1H), 7.40 (m, 2H), 7.59 (d, J=7 Hz, 1H), 8.20 (d, J=7 Hz, 1H), 10.84 (br s, 1H).

### Example 56

### 2-(4-Cyclopropylpiperazin-1-yl)-6-trifluoromethylquinoline hydrochloride

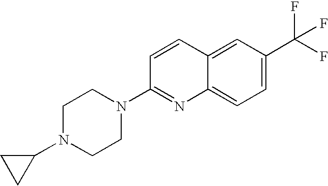

¹H NMR (DMSO-d₆) δ0.81 (m, 2H), 1.19 (br s, 2H), 2.88 (br s, 1H), 3.20-3.70 (m, 6H), 4.73 (m, 2H), 7.52 (d, J=8 Hz, 1H), 7.86 (m, 2H), 8.26 (br s, 1H), 8.33 (d, J=8 Hz, 1H), 11.12 (br s, 1H); HPLC-MS: m/z 322 (MH⁺); Rf: 2.41 min.

### Example 57

### 2-(4-Cyclopropylpiperazin-1-yl)-6-propylquinoline hydrochloride

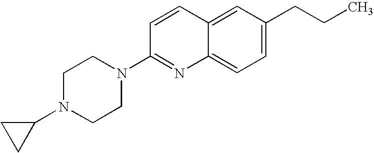

¹H NMR (DMSO-d₆) δ0.82 (m, 2H), 0.91 (t, J=7 Hz, 3H), 1.18 (br s, 2H), 1.66 (sext, J=7 Hz, 2H), 2.69 (t, J=7 Hz, 2H), 2.85 (br s, 1H), 3.30-3.75 (m, 6H), 4.72 (m, 2H), 7.51 (m, 1H), 7.62 (m, 1H), 7.69 (br s, 1H), 7.97 (br s, 1H), 8.33 (br s, 1H), 11.20 (br s, 1H); HPLC-MS: m/z 296 (MH⁺); Rf: 1.97 min.

### Example 58

### 2-(4-Ethylpiperazin-1-yl)quinoline hydrochloride

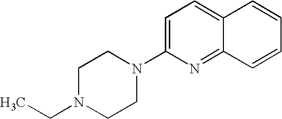

Reference can also be made to S. Cacchi et al., SynLett 1997, 1400-1402.

¹H NMR (DMSO-d₆) δ1.30 (t, J=7 Hz, 3H), 3.15 (m, 4H), 3.55-3.85 (m, 4H), 4.81 (m, 2H), 7.47 (m, 1H), 7.53 (m, 1H), 7.74 (m, 1H), 7.89 (d, J=8 Hz, 1H), 8.13 (br s, 1H), 8.40 (br s, 1H), 11.34 (br s, 1H); HPLC-MS: m/z 242 (MH⁺); Rf: 1.04 min.

### Example 59

### General Procedure (B)

### 3-(4-Isopropyl-piperazin-1-yl)-6-phenyl-pyridazine, hydrochloride

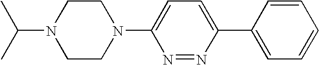

This compound was prepared according to General Procedure (B), starting from 1-isopropylpiperazine and 3-chloro-6-phenylpyridazine, prepared as described in J. Heterocycl. Chem., 15, 881 (1978).

¹H NMR (D₂O): δ1.46 (d, 6H); 3.28 (m, 2H); 3.48 (m, 2H); 3.64-3.84 (m, 3H); 4.57 (m, 2H); 7.63-7.72 (m, 4H); 7.90 (m, 2H); 8.12 (d, 1H); HPLC-MS: m/z=283.2 (M+1); Rₜ=1.52 min.

### Example 60

### General Procedure (B)

### 3-(4-Cyclopentyl-piperazin-1-yl)-6-(4-methanesulfonyl-phenyl)-pyridazine

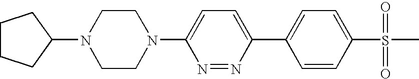

This compound was prepared according to General Procedure (B), starting from 1-cyclopentylpiperazine and 3-chloro-6-(4-methanesulfonyl-phenyl)-pyridazine, prepared as described in J. Heterocycl. Chem., 15, 881 (1978).

¹H NMR (CDCl₃): δ1.38-1.80 (m, 6H), 1.92 (m, 2H); 2.56 (quint, 1H); 2.66 (dd, 4H); 3.10 (s, 3H); 3.97 (dd, 4H); 6.99 (d, 1H); 7.69 (d, 1H); 8.03 (d, 2H); 8.20 (d, 2H); HPLC-MS: m/z=387.0 (M+1); Rₜ=2.20 min.

### Example 61

### General Procedure (B)

### 3-(4-Cyclopropylmethyl-piperazin-1-yl)-6-(4-methanesulfonyl-phenyl)-pyridazine

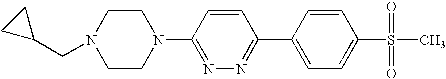

This compound was prepared according to General Procedure (B), starting from 1-cyclopentylpiperazine and 3-chloro-6-(4-methanesulfonyl-phenyl)-pyridazine, prepared as described in J. Heterocycl. Chem., 15, 881 (1978).

¹H NMR (CDCl₃): δ0.15 (q, 2H); 0.57 (m, 2H); 0.92 (m, 1H); 2.34 (d, 2H); 2.69 (dd, 4H); 3.10 (s, 3H); 3.80 (dd, 4H); 7.01 (d, 1H); 7.70 (d, 1H); 8.03 (d, 2H); 8.21 (d, 2H); HPLC-MS: m/z=373.4 (M+1); Rₜ=2.04 min.

### Example 62

### General Procedure (B)

### 3-(4-Isopropyl-piperazin-1-yl)-6-(4-methanesulfonyl-phenyl)-pyridazine

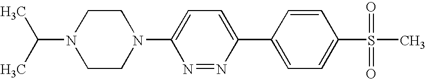

This compound was prepared according to General Procedure (B), starting from 1-isopropylpiperazine and 3-chloro-6-(4-methanesulfonyl-phenyl)-pyridazine, prepared as described in J. Heterocycl. Chem., 15, 881 (1978).

¹H NMR (DMSO-d₆): δ1.01 (d, 6H); 2.57 (m, 4H); 2.71 (m, 1H); 3.26 (s, 3H); 3.67 (m, 4H); 7.39 (d, 1H); 8.02 (d, 2H); 8.06 (d, 1H); 8.30 (d, 2H); HPLC-MS: m/z=360.8 (M+1); Rₜ=1.43 min.

### Example 63

### General Procedure (B)

### 3-(4-Chloro-phenyl)-6-(4-isopropyl-piperazin-1-yl)-4-methyl-pyridazine, dihydrochloride

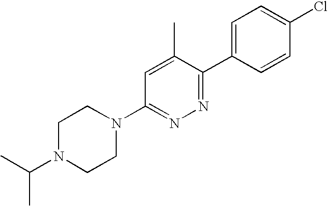

This compound was prepared according to General Procedure (B), starting from 1-isopropylpiperazine and 6-chloro-3-(4-chloro-phenyl)-4-methyl-pyridazine, prepared as described in J. Heterocycl. Chem., 15, 881 (1978).

¹H NMR (D₂O): δ1.08 (d, 6H); 2.10 (s, 1H); 3.01 (m, 2H); 3.23 (m, 2H); 3.28-3.44 (m, 3H); 4.31 (broad d, 2H); 7.27 (d, 2H); 7.34 (d, 2H); 7.58 (s, 1H); HPLC-MS: m/z=331.1 (M+1); Rₜ=3.1 min. C18 H23 N4 Cl, 2 HCl

Calc.: C, 53.54; H, 6.24; N, 13.88 Found: C, 53.34; H, 6.31; N, 13.70.

### Example 64

### General Procedure (B)

### 3-(4-Chloro-phenyl)-6-(4-cyclopentyl-piperazin-1-yl)-4-methyl-pyridazine, hydrochloride

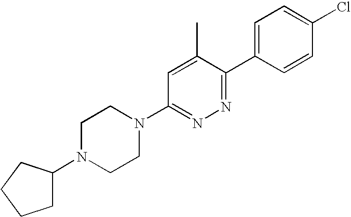

This compound was prepared according to General Procedure (B), starting from 1-cyclopentylpiperazine and 6-chloro-3-(4-chloro-phenyl)-4-methyl-pyridazine, prepared as described in J. Heterocycl. Chem., 15, 881 (1978).

¹H NMR (D₂O): δ1.29-1.60 (m, 6H); 1.91 (m, 2H); 2.12 (s, 3H); 3.00 (m, 2H); 3.24 (m, 2H); 3.36 (m, 1H); 3.51 (broad d, 2H); 4.29 (broad d, 2H); 7.29 (d, 2H); 7.36 (d, 2H); 7.60 (s, 1H); HPLC-MS: m/z=357.1 (M+1); Rₜ=3.25 min. C20 H25 N4 Cl, 2 HCl

Calc.: C, 55.89; H, 6.33; N, 13.04. Found: C, 55.83; H, 6.47; N, 12.93.

### Example 65

### General Procedure (B)

### 3-(4-Chlorophenyl)-6-(4-cyclopentylpiperazin-1-yl)-pyridazine

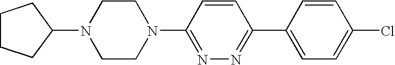

This compound was prepared as described in Example 6, starting from 1-cyclopentylpiperazine and 3-chloro-6-(4-chlorophenyl)-pyridazine, prepared as described in J. Heterocycl. Chem., 15, 881 (1978). The title compound was obtained as the free base.

¹H NMR (CDCl₃): δ1.39-1.81 (m, 6H), 1.91 (m, 2H), 2.56 (q, 1H), 2.66 (dd, 4H), 3.74 (dd, 4H), 6.96 (d, J=9.5 Hz, 1H), 7.43 (d, J=8.7 Hz, 2H), 7.61 (d, J=9.5 Hz, 1H), 7.93 (d, J=8.7 Hz, 2H); HPLC-MS (Method #): m/z=343 (M+1); Rₜ=2.93 min.

### Example 66

### General Procedure (B)

### 3-(4-Cyclopentylpiperazin-1-yl)-6-(3-fluoro-4-methoxyphenyl)-pyridazine, dihydrochloride

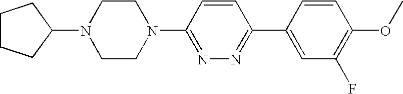

This compound was prepared as described in Example 6, starting from 1-cyclopentylpiperazine and 3-chloro-6-(3-fluoro-4-methoxyphenyl)-pyridazine, prepared as described in J. Heterocycl. Chem., 15, 881 (1978).

¹H NMR (DMSO-d₆): δ1.45-2.15 (m, 8H), 3.17 (m, 2H), 3.40-3.77 (m, 5H), 3.92 (s, 3H), 7.34 (t, J=8.7 Hz, 1H), 7.80 (d, J=9.8 Hz, 1H), 7.85-8.05 (m, 2H), 8.29 (d, J=9.8 Hz, 1H), 11.75 (bs, 1H); HPLC-MS: m/z=357 (M+1); Rₜ=2.47 min.

### Example 67

### General Procedure (B)

### 3-(4-Cyclopentylpiperazin-1-yl)-6-(3,4-dimethoxyphenyl)-pyridazine

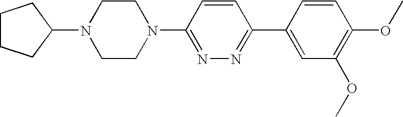

This compound was prepared as described in Example 6, starting from 1-cyclopentylpiperazine and 3-chloro-6-(3,4-dimethoxyphenyl)-pyridazine, prepared as described in J. Heterocycl. Chem., 15, 881 (1978). The title compound was obtained as the free base.

¹H NMR (CDCl₃): δ1.40-1.65 (m, 4H), 1.73 (m, 2H), 1.91 (m, 2H), 2.55 (q, 1H), 2.66 (t, 4H), 3.72 (t, 4H), 3.93 (s, 3H), 3.98 (s, 3H), 6.93 (d, 1H), 6.97 (d, 1H), 7.36 (dd, 1H), 7.64 (d, 1H), 7.86 (d, 1H); HPLC-MS: m/z=370 (M+1); Rₜ=1.90 min.

### Example 68

### General Procedure (B)

### 3-(4-Chlorophenyl)-6-(4-cyclopropylmethylpiperazin-1-yl)-pyridazine

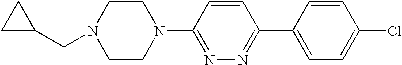

This compound was prepared as described in Example 6, starting from 1-cyclopropylmethylpiperazine and 3-chloro-6-(4-chlorophenyl)-pyridazine, prepared as described in J. Heterocycl. Chem., 15, 881 (1978). The title compound was obtained as the free base.

¹H NMR (CDCl₃): δδ0.46 (m, 2H), 0.88 (m, 2H), 1.33 (m, 1H), 2.90 (d, 2H), 3.1-3.5 (m, 4H), 4.1-4.35 (m, 4H), 7.05 (d, 1H), 7.46 (d, 2H), 7.72 (d, 1H), 7.95 (d, 2H); HPLC-MS: m/z=329 (M+1); Rₜ=2.11 min.

### Example 69

### General Procedure (B)

### Name

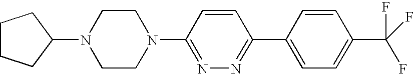

This compound was prepared as described in Example 6, starting from 1-cyclopentylpiperazine and 3-chloro-6-(4-trifluoromethylphenyl)-pyridazine, prepared as described in J. Heterocycl. Chem., 15, 881 (1978). The title compound was obtained as the free base.

¹H NMR (CDCl₃): δ1.40-1.65 (m, 4H), 1.65-1.80 (m, 2H), 1.92 (m, 2H), 2.55 (q, 1H), 2.65 (t, 4H), 3.76 (t, 4H), 6.99 (d, 1H), 7.67 (d, 1H), 7.72 (d, 2H), 8.12 (d, 2H); HPLC-MS): m/z=377 (M+1); Rₜ=2.68 min.

### Example 70

### General Procedure (B)

### 3-(4-Isopropylpiperazin-1-yl)-6-(4-trifluoromethylphenyl)-pyridazine

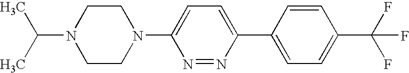

This compound was prepared as described in Example 6, starting from 1-isopropylpiperazine and 3-chloro-6-(4-trifluoromethylphenyl)-pyridazine, prepared as described in J. Heterocycl. Chem., 15, 881 (1978). The title compound was obtained as the free base.

¹H NMR (DMSO-d₆): δ1.20 (d, 6H), 2.8-4.2 (m, 9H), 7.47 (d, 1H), 7.85 (d, 2H), 8.12 (d, 1H), 8.28 (d, 2H); HPLC-MS: m/z=351 (M+1); Rₜ=2.51 min.

### Example 71

### General Procedure (B)

### 3-(4-Cyclopropylmethylpiperazin-1-yl)-6-(4-trifluoromethylphenyl)-pyridazine

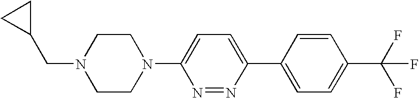

This compound was prepared as described in Example 6, starting from 1-cyclopropylmethylpiperazine and 3-chloro-6-(4-trifluoromethylphenyl)-pyridazine, prepared as described in J. Heterocycl. Chem., 15, 881 (1978). The title compound was obtained as the free base.

¹H NMR (CDCl₃): δ0.15 (m, 2H), 0.57 (m, 2H), 0.92 (m, 1H), 2.33 (d, 2H), 2.69 (t, 4H), 3.79 (t, 4H), 7.00 (d, 1H), 7.67 (d, 1H), 7.72 (d, 2H), 8.12 (d, 2H); HPLC-MS: m/z=363 (M+1); Rₜ=2.65 min.

### Example 72

### General Procedure (B)

### 3-(4-Chlorophenyl)-6-(4-isopropylpiperazin-1-yl)-pyridazine

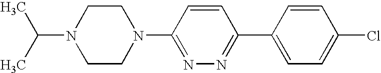

This compound was prepared as described in Example 6, starting from 1-isopropylpiperazine and 3-chloro-6-(4-chlorophenyl)-pyridazine, prepared as described in J. Heterocycl. Chem., 15, 881 (1978). The title compound was obtained as the free base.

¹H NMR (CDCl₃): δ1.10 (d, 6H), 2.68 (t, 4H), 2.75 (q, 1H), 3.73 (t, 4H), 6.97 (d, 1H), 7.43 (d, 2H), 7.61 (d, 1H), 7.94 (d, 2H); HPLC-MS: m/z=317 (M+1); Rₜ=2.03 min.

### Example 73

### General Procedure (B)

### 3-(4-Cyclopropylmethylpiperazin-1-yl)-6-(3-fluoro-4-methoxyphenyl)-pyridazine

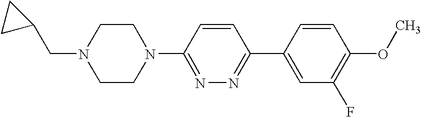

This compound was prepared as described in Example 6, starting from 1-cyclopropylmethylpiperazine and 3-chloro-6-(3-fluoro-4-methoxyphenyl)-pyridazine, prepared as described in J. Heterocycl. Chem., 15, 881 (1978). The title compound was obtained as the free base.

¹H NMR (DMSO-d₆): δ0.40 (m, 2H), 0.65 (m, 2H), 1.15 (m, 1H), 2.8-3.7 (m, 10H), 3.90 (s, 3H), 7.29 (t, 1H), 7.47 (d, 1H), 7.88 (d, 1H), 7.93 (d, 1H), 8.06 (d, 1H); HPLC-MS: m/z=343 (M+1); Rₜ=1.90 min.

### Example 74

### General Procedure (B)

### 3-(3-Fluoro-4-methoxyphenyl)-6-(4-isopropylpiperazin-1-yl)-pyridazine

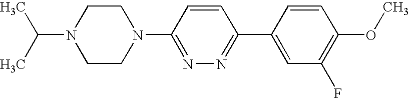

This compound was prepared as described in Example 6, starting from 1-isopropylpiperazine and 3-chloro-6-(3-fluoro-4-methoxyphenyl)-pyridazine, prepared as described in J. Heterocycl. Chem., 15, 881 (1978). The title compound was obtained as the free base.

¹H NMR (CDCl₃): δ1.11 (d, 6H), 2.70 (m, 4H), 2.80 (q, 1H), 3.74 (m, 4H), 3.94 (s, 3H), 6.96 (d, 1H), 7.04 (t, 1H), 7.57 (d, 1H), 7.72 (d, 1H), 7.78 (m, 1H); HPLC-MS: m/z=331 (M+1); Rₜ=1.57 min.

### Example 75

### General Procedure (B)

### 3-(3,4-Dimethoxyphenyl)-6-(4-isopropylpiperazin-1-yl)-pyridazine, dihydrochloride

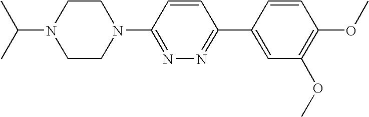

This compound was prepared as described in Example 6, starting from 1-isopropylpiperazine and 3-chloro-6-(3,4-dimethoxyphenyl)-pyridazine, prepared as described in J. Heterocycl. Chem., 15, 881 (1978).

¹H NMR (DMSO-d₆): δ1.32 (d, 6H), 3.17 (q, 1H), 3.3-4.1 (m, 6H), 3.84 (s, 3H), 3.87 (s, 3H), 4.56 (d, 2H), 7.09 (d, 1H), 7.62 (d, 1H), 7.68-7.73 (m, 2H), 8.23 (d, 1H), 11.35 (s, 1H); HPLC-MS: m/z=343 (M+1); Rₜ=1.50 min.

### Example 76

### General Procedure (B)

### (9a-R)-2-(6-Trifluoromethoxyquinolin-2-yl)octahydropyrido[1,2-a]pyrazine hydrochloride

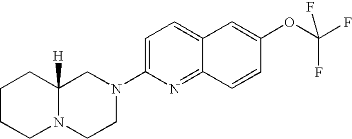

This compound was prepared using the General Procedure (B) from (9a-R)-octahydropyrido[1,2-a]pyrazine and 2-chloro-6-trifluoromethoxyquinoline.

¹H NMR (DMSO-d₆) δ1.40-1.55 (m, 1H), 1.65-2.08 (m, 5H), 2.93 (m, 1H), 3.18 (m, 1H), 3.25-3.55 (m, 4H), 3.71 (m, 1H), 4.85 (m, 2H), 7.61 (d, J=8 Hz, 1H), 7.70 (d, J=8 Hz, 1H), 7.93 (s, 1H), 8.19 (br s, 1H), 8.39 (d, J=8 Hz, 1H), 11.60 (br s, 1H); HPLC-MS: m/z 352 (MH⁺); Rt=2.67 min.

### Example 77

### General Procedure (B)

### 7-Fluoro-2-(4-isopropylpiperazin-1-yl)-6-methylquinoline hydrochloride

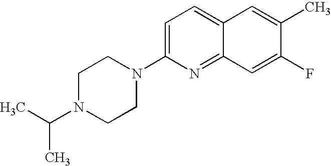

This compound was prepared using the General Procedure (B) from 1-isopropylpiperazine and 2-chloro-7-fluoro-6-methylquinoline.

¹H NMR (DMSO-d₆) δ1.32 (d, J=7 Hz, 6H), 2.36 (s, 3H), 3.26 (m, 2H), 3.54 (m, 3H), 3.83 (m, 2H), 4.88 (br s, 2H), 7.49 (d, J=8 Hz, 1H), 7.85 (d, J=8 Hz, 1H), 8.09 (br s, 1H), 8.35 (d, J=8 Hz, 1H), 11.57 (br s, 1H); HPLC-MS: m/z 287 (MH⁺); Rt=1.47 min.

### Example 78

### General Procedure (B)

### 7-Chloro-2-(4-isopropylpiperazin-1-yl)quinoline hydrochloride

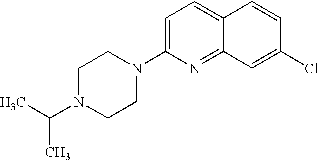

This compound was prepared using the General Procedure (B) from 1-isopropylpiperazine and 2,7-dichloroquinoline.

¹H NMR (DMSO-d₆) δ1.31 (d, J=7 Hz, 6H), 3.23 (m, 2H), 3.53 (m, 3H), 3.79 (m, 2H), 4.87 (br s, 2H), 7.48 (d, J=8 Hz, 1H), 7.54 (d, J=8 Hz, 1H), 7.92 (d, J=8 Hz, 1H), 8.26 (br s, 1H), 8.38 (d, J=8 Hz, 1H), 11.50 (br s, 1H); HPLC-MS: m/z 289 (MH⁺); Rt=1.61 min.

### Example 79

### General Procedure (B)

### 6-Fluoro-2-(4-isopropylpiperazin-1-yl)quinoline hydrochloride

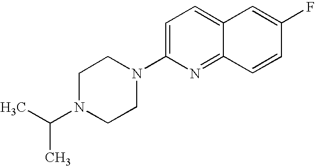

This compound was prepared using the General Procedure (B) from 1-isopropylpiperazine and 2-chloro-6-fluoroquinoline.

¹H NMR (DMSO-d₆) δ1.31 (d, J=7 Hz, 6H), 3.22 (m, 2H), 3.53 (m, 3H), 3.79 (m, 2H), 4.85 (br s, 2H), 7.55-7.70 (m, 2H), 7.45 (d, J=8 Hz, 1H), 8.26 (br s, 1H), 8.36 (J=8 Hz, 1H), 11.52 (br s, 1H); HPLC-MS: m/z 274 (MH⁺); Rt=1.21 min.

### Example 80

### General Procedure (B)

### 2-(4-Cyclopropylpiperazin-1-yl)-7-fluoro-6-methylquinoline hydrochloride

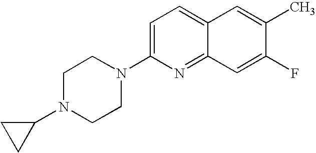

This compound was prepared using the General Procedure (B) from 1-cyclopropylpiperazine and 2-chloro-7-fluoro-6-methylquinoline.

¹H NMR (DMSO-d₆) δ0.82 (m, 2H), 1.20 (m, 2H), 2.36 (s, 3H), 2.87 (m, 1H), 3.25-4.15 (m, 6H), 4.74 (br s, 2H), 7.43 (d, J=8 Hz, 1H), 7.80 (m, 2H), 8.28 (d, J=8 Hz, 1H), 11.36 (br s, 1H); HPLC-MS: m/z 287 (MH⁺); Rt=1.47 min.

### Example 81

### General Procedure (B)

### 2-(4-Cyclopropylpiperazin-1-yl)-7-fluoro-6-methoxyquinoline hydrochloride

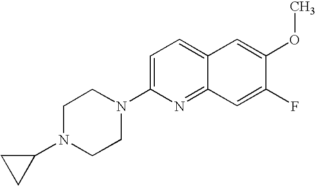

This compound was prepared using the General Procedure (B) from 1-cyclopropylpiperazine and 2-chloro-7-fluoro-6-methoxyquinoline.

¹H NMR (DMSO-d₆) δ0.82 (m, 2H), 1.18 (m, 2H), 2.88 (m, 1H), 3.25-4.10 (m, 6H), 3.93 (s, 3H), 4.64 (br s, 2H), 7.40 (d, J=8 Hz, 1H), 7.55 (d, J=8 Hz, 1H), 7.73 (br s, 1H), 8.24 (d, J=8 Hz, 1H), 11.11 (br s, 1H); HPLC-MS: m/z 301 (MH⁺); Rt=1.37 min.

### Example 82

### General Procedure (B)

### 7-Fluoro-2-(4-isopropylpiperazin-1-yl)-6-methoxyquinoline hydrochloride

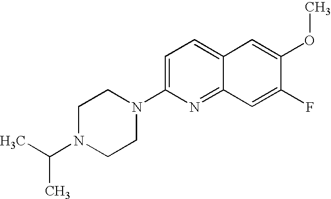

This compound was prepared using the General Procedure (B) from 1-isopropylpiperazine and 2-chloro-7-fluoro-6-methoxyquinoline.

¹H NMR (DMSO-d₆) δ1.31 (d, J=7 Hz, 6H), 3.16 (m, 2H), 3.45-4.05 (m, 5H), 3.93 (s, 3H), 4.72 (m, 2H), 7.42 (d, J=8 Hz, 1H), 7.56 (d, J=8 Hz, 1H), 7.83 (br s, 1H), 8.25 (d, J=8 Hz, 1H), 11.13 (br s, 1H); HPLC-MS: m/z 303 (MH⁺); Rt=1.41 min.

### Example 83

### General Procedure (B)

### (9a-R)-2-(7-Fluoro-6-methoxyquinolin-2-yl)octahydropyrido[1,2-a]pyrazine hydrochloride

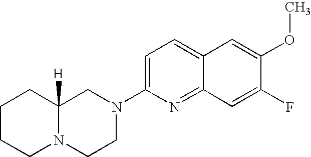

This compound was prepared using the General Procedure (B) from (9a-R)-octahydropyrido[1,2-a]pyrazine and 2-chloro-7-fluoro-6-methoxyquinoline.

¹H NMR (DMSO-d₆) δ1.40-1.55 (m, 1H), 1.65-2.08 (m, 5H), 2.93 (m, 1H), 3.20 (m, 1H), 3.25-3.55 (m, 4H), 3.73 (m, 1H), 4.81 (m, 2H), 7.50 (d, J=8 Hz, 1H), 7.62 (d, J=8 Hz, 1H), 8.13 (br s, 1H), 8.34 (d, J=8 Hz, 1H), 11.59 (br s, 1H); HPLC-MS: m/z 315 (MH⁺); Rt=1.41 min.

### Example 84

### General Procedure (B)

### (9a-R)-2-(6-Trifluoromethylquinolin-2-yl)octahydropyrido[1,2-]pyrazine hydrochloride

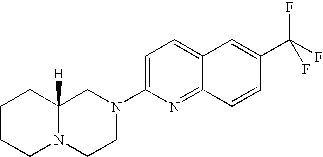

This compound was prepared using the General Procedure (B) from (9a-R)-octahydropyrido[1,2-a]pyrazine and 2-chloro-6-trifluoromethylquinoline.

¹H NMR (DMSO-d₆) δ1.40-1.55 (m, 1H), 1.65-2.05 (m, 5H), 2.92 (m, 1H), 3.20 (m, 1H), 3.30-3.55 (m, 4H), 3.70 (m, 1H), 4.88 (m, 2H), 7.61 (d, J=8 Hz, 1H), 7.93 (d, J=8 Hz, 1H), 8.15 (br s, 1H), 8.31 (s, 1H), 8.43 (d, J=8 Hz, 1H), 11.60 (br s, 1H); HPLC-MS: m/z 335 (MH⁺); Rt=2.27 min.

### Example 85

### General Procedure (B)

### 7-Fluoro-2-(4-isopropylpiperazin-1-yl)quinoline hydrochloride

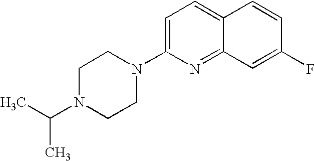

This compound was prepared using the General Procedure (B) from 1-isopropylpiperazine and 2-chloro-7-fluoroquinoline.

¹H NMR (DMSO-d₆) δ1.31 (d, J=7 Hz, 6H), 3.19 (m, 2H), 3.45-4.20 (m, 5H), 4.84 (m, 2H), 7.32 (m, 1H), 7.45 (d, J=8 Hz, 1H), 7.83 (br s, 1H), 7.95 (m, 1H), 8.35 (m, 1H), 11.35 (br s, 1H); HPLC-MS: m/z 274 (MH⁺); Rt=1.31 min.

### Example 86

### General Procedure (B)

### 6-Chloro-2-(4-isopropylpiperazin-1-yl)quinoline hydrochloride

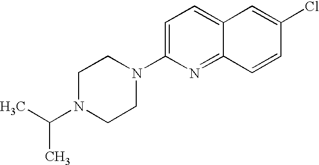

This compound was prepared using the General Procedure (B) from 1-isopropylpiperazine and 2,6-dichloroquinoline.

¹H NMR (DMSO-d₆) δ1.31 (d, J=7 Hz, 6H), 3.19 (m, 2H), 3.45-3.80 (m, 5H), 4.82 (m, 2H), 7.55 (d, J=8 Hz, 1H), 7.72 (d, J=8 Hz, 1H), 7.99 (s, 1H), 8.07 (br s, 1H), 8.29 (d, J=8 Hz, 1H), 11.38 (br s, 1H); HPLC-MS: m/z 290 (MH⁺); Rt=1.64 min.

### Example 87

### General Procedure (B)

### 6-Isopropyl-2-(4-isopropylpiperazin-1-yl)quinoline hydrochloride

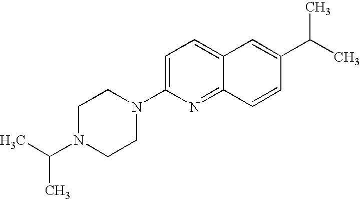

This compound was prepared using the General Procedure (B) from 1-isopropylpiperazine and 2-chloro-6-isopropylquinoline.

¹H NMR (DMSO-d₆) δ1.27 (d, J=7 Hz, 6H), 1.32 (d, J=7 Hz, 6H), 3.05 (sept, J=7 Hz, 1H), 3.26 (m, 2H), 3.40-3.95 (m, 5H), 4.86 (m, 2H), 7.55 (d, J=8 Hz, 1H), 7.72 (d, J=8 Hz, 1H), 7.77 (s, 1H), 8.21 (br s, 1H), 8.42 (d, J=8 Hz, 1H), 11.55 (br s, 1H); HPLC-MS: m/z 298 (MH⁺); Rt=1.87 min.

### Example 88

### General Procedure (B)

### 2-(4-Cyclopropylpiperazin-1-yl)-6-isopropylquinoline hydrochloride

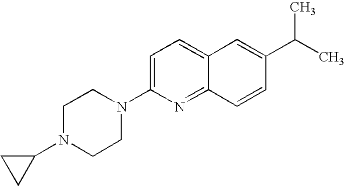

This compound was prepared using the General Procedure (B) from 1-cyclopropylpiperazine and 2-chloro-6-isopropylquinoline.

¹H NMR (DMSO-d₆) δ0.83 (m, 2H), 1.20 (m, 2H), 1.27 (d, J=7 Hz, 6H), 2.86 (br s, 1H), 3.04 (sept, J=7 Hz, 1H), 3.25-3.85 (m, 5H), 4.15 (br s, 1H), 4.74 (m, 2H), 7.53 (m, 1H), 7.70 (d, J=8 Hz, 1H), 7.75 (s, 1H), 8.06 (br s, 1H), 8.38 (m, 1H), 11.41 (br s, 1H); HPLC-MS: m/z (MH⁺); Rt=min.

### Example 89

### General Procedure (B)

### 2-(4-Cyclopropylpiperazin-1-yl)quinoline hydrochloride

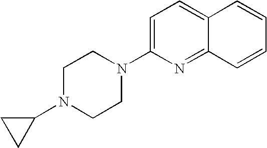

This compound was prepared using the General Procedure (B) from 1-cyclopropylpiperazine and 2-chloroquinoline.

¹H NMR (DMSO-d₆) δ0.83 (m, 2H), 1.19 (m, 2H), 2.87 (br s, 1H), 3.30-3.80 (m, 6H), 4.74 (m, 2H), 7.45 (m, 1H), 7.52 (d, J=8 Hz, 1H), 7.73 (m, 1H), 7.89 (d, J=8 Hz, 1H), 8.02 (br s, 1H), 8.38 (m, 1H), 11.22 (br s, 1H); HPLC-MS: m/z (MH⁺); Rt=min.

### Example 90

### General Procedure (B)

### 2-(4-Cyclopropylpiperazin-1-yl)-6,7-dimethoxyquinoline hydrochloride

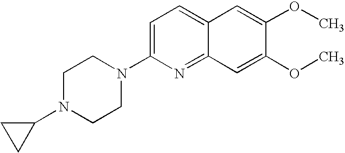

This compound was prepared using the General Procedure (B) from 1-cyclopropylpiperazine and 2-chloro-6,7-dimethoxyquinoline.

¹H NMR (DMSO-d₆) δ0.82 (m, 2H), 1.20 (m, 2H), 2.87 (br s, 1H), 3.25-3.75 (m, 6H), 3.86 (s, 3H), 3.90 (s, 3H), 4.66 (m, 2H), 7.25-7.50 (m, 3H), 8.24 (br s, 1H), 11.38 (br s, 1H); HPLC-MS: m/z 314 (MH⁺); Rt=1.27 min.

### Example 91

### General Procedure (B)

### 2-(4-Isopropylpiperazin-1-yl)-6,7-dimethoxyquinoline hydrochloride

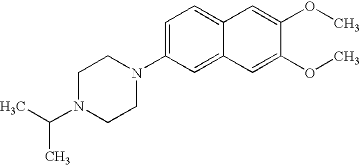

This compound was prepared using the General Procedure (B) from 1-isopropylpiperazine and 2-chloro-6,7-dimethoxyquinoline.

¹H NMR (DMSO-d₆) δ1.32 (d, J=7 Hz, 6H), 3.25 (m, 2H), 3.45-4.00 (m, 5H), 3.87 (s, 3H), 3.91 (s, 3H), 4.80 (m, 2H), 7.39 (m, 2H), 7.96 (br s, 1H), 8.34 (br s, 1H), 11.50 (br s, 1H); HPLC-MS: m/z 316 (MH⁺); Rt=1.27 min.

### Example 92

### General Procedure (B)

### 2-(4-Cyclopropylpiperazin-1-yl)-7-fluoroquinoline hydrochloride

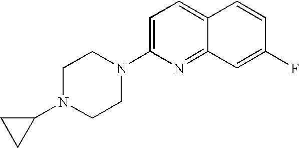

This compound was prepared using the General Procedure (B) from 1-cyclopropylpiperazine and 2-chloro-7-fluoroquinoline.

¹H NMR (DMSO-d₆) δ0.82 (m, 2H), 1.22 (m, 2H), 2.87 (br s, 1H), 3.30-3.80 (m, 6H), 4.79 (m, 2H), 7.34 (m, 1H), 7.47 (d, J=8 Hz, 1H), 7.85 (br s, 1H), 7.96 (m, 1H), 8.37 (m, 1H), 11.55 (br s, 1H); HPLC-MS: m/z 271 (MH⁺); Rt=1.24 min.

### Example 93

### General Procedure (B)

### 2-(4-Cyclopropylpiperazin-1-yl)6,8-difluoroquinoline hydrochloride

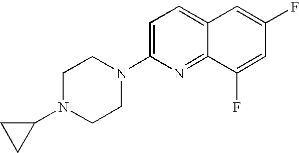

This compound was prepared using the General Procedure (B) from 1-cyclopropylpiperazine and 2-chloro-6,8-difluoroquinoline.

¹H NMR (DMSO-d₆) δ0.82 (m, 2H), 1.19 (m, 2H), 2.87 (br s, 1H), 3.20-3.70 (m, 6H), 4.63 (m, 2H), 7.48 (m, 3H), 8.19 (d, J=8 Hz, 1H), 11.11 (br s, 1H); HPLC-MS: m/z 290 (MH⁺); Rt=2.27 min.

### Example 94

### General Procedure (B)

### 2-(4-Cyclopropylpiperazin-1-yl)-6-fluoroquinoline hydrochloride

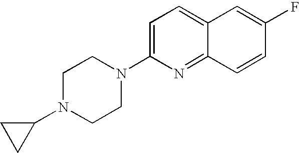

This compound was prepared using the General Procedure (B) from 1-cyclopropylpiperazine and 2-chloro-6-fluoroquinoline.

¹H NMR (DMSO-d₆) δ0.82 (m, 2H), 1.21 (m, 2H), 2.87 (br s, 1H), 3.30-3.80 (m, 6H), 4.73 (m, 2H), 7.56 (d, J=8 Hz, 1H), 7.62 (m, 1H), 7.71 (d, J=8 Hz, 1H), 8.09 (br s, 1H), 8.33 (d, J=8 Hz, 1H), 11.42 (br s, 1H); HPLC-MS: m/z 272 (MH⁺); Rt=1.27 min.

### Example 95

### General Procedure (B)

### 7-Chloro-2-(4-cyclopropylpiperazin-1-yl)quinoline hydrochloride

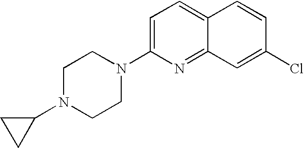

This compound was prepared using the General Procedure (B) from 1-cyclopropylpiperazine and 2,7-dichloroquinoline.

¹H NMR (DMSO-d₆) δ0.82 (m, 2H), 1.20 (m, 2H), 2.87 (br s, 1H), 3.25-3.75 (m, 6H), 4.7 (m, 2H), 7.40 (d, J=8 Hz, 1H), 7.46 (d, J=8 Hz, 1H), 7.87 (d, J=8 Hz, 1H), 7.94 (br s, 1H), 8.29 (d, J=8 Hz, 1H), 11.29 (br s, 1H); HPLC-MS: m/z 288 (MH⁺); Rt=1.71 min.

### Example 96

### General Procedure (B)

### (9a-R)-2-Quinolin-2-yl-octahydropyrido[1,2-a]pyrazine hydrochloride

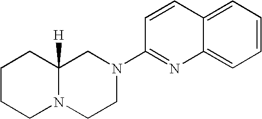

This compound was prepared using the General Procedure (B) from (9a-R)-octahydropyrido[1,2-a]pyrazine and 2-chloroquinoline.

¹H NMR (DMSO-d₆) δ1.40-1.55 (m, 1H), 1.65-2.10 (m, 5H), 2.92 (m, 1H), 3.25 (m, 1H), 3.35-3.90 (m, 5H), 4.90 (m, 2H), 7.49 (m, 1H), 7.59 (d, J=8 Hz, 1H), 7.77 (m, 1H), 7.92 (d, J=8 Hz, 1H), 8.32 (br s, 1H), 8.46 (m, 1H), 11.69 (br s, 1H); HPLC-MS: m/z 268 (MH⁺); Rt=1.07 min.

### Example 97

### General Procedure (B)

### (9a-R)-2-(6-Chloroquinolin-2-yl)octahydropyrido[1,2-a]pyrazine hydrochloride

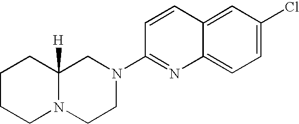

This compound was prepared using the General Procedure (B) from (9a-R)-octahydropyrido[1,2-a]pyrazine and 2,6-dichloroquinoline.

¹H NMR (DMSO-d₆) δ1.40-1.55 (m, 1H), 1.65-2.05 (m, 5H), 2.93 (m, 1H), 3.18 (m, 1H), 3.25-3.55 (m, 4H), 3.68 (m, 1H), 4.90 (m, 2H), 7.56 (d, J=8 Hz, 1H), 7.71 (d, J=8 Hz, 1H), 7.98 (s, 1H), 8.07 (br s, 1H), 8.29 (d, J=8 Hz, 1H), 11.47 (br s, 1H); HPLC-MS: m/z 302 (MH⁺); Rt=1.81 min.

### Example 98

### General Procedure (B)

### (9a-R)-2-(7-Fluoro-6-methylquinolin-2-yl)octahydropyrido[1,2-a]pyrazine hydrochloride

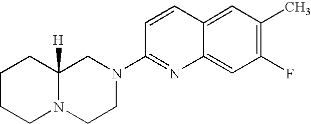

This compound was prepared using the General Procedure (B) from (9a-R)-octahydropyrido[1,2-a]pyrazine and 2-chloro-7-fluoro-6-methylquinoline.

¹H NMR (DMSO-d₆) δ1.40-1.55 (m, 1H), 1.65-2.10 (m, 5H), 2.36 (s, 3H), 2.92 (m, 1H), 3.23 (m, 1H), 3.30-3.60 (m, 4H), 3.80 (m, 1H), 4.87 (m, 2H), 7.51 (d, J=8 Hz, 1H), 7.84 (d, J=8 Hz, 1H), 8.13 (br s, 1H), 8.36 (d, J=8 Hz, 1H), 11.77 (br s, 1H); HPLC-MS: m/z 300 (MH⁺); Rt=1.54 min.

### Example 99

### General Procedure (B)

### (9a-R)-2-(6-Propylquinolin-2-yl)octahydropyrido[1,2-a]pyrazine hydrochloride

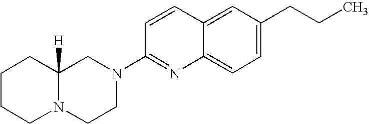

This compound was prepared using the General Procedure (B) from (9a-R)-octahydropyrido[1,2-a]pyrazine and 2-chloro-6-propylquinoline.

¹H NMR (DMSO-d₆) δ0.91 (t, J=7 Hz, 3H), 1.40-1.55 (m, 1H), 1.60-2.10 (m, 7H), 2.70 (t, J=7 Hz, 2H), 2.92 (m, 1H), 3.24 (m, 1H), 3.30-3.85 (m, 5H), 4.88 (m, 2H), 7.57 (d, J=8 Hz, 1H), 7.65 (d, J=8 Hz, 1H), 7.72 (s, 1H), 8.28 (br s, 1H), 8.41 (m, 1H), 11.69 (br s, 1H); HPLC-MS: m/z 309 (MH⁺); Rt=2.27 min.

### Example 100

### General Procedure (B)

### 2-(4-Isopropylpiperazin-1-yl)quinoxaline hydrochloride

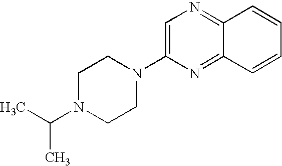

This compound was prepared using the General Procedure (B) from 1-isopropylpiperazine and 2-chloroquinoxaline.

¹H NMR (DMSO-d₆) δ1.31 (d, J=7 Hz, 6H), 3.13 (m, 2H), 3.45-3.65 (m, 5H), 4.75 (m, 2H), 7.48 (m, 1H), 7.66 (m, 2H), 7.88 (d, J=8 Hz, 1H), 8.91 (s, 1H), 11.15 (br s, 1H); HPLC-MS: m/z 257 (MH⁺); Rt=1.25 min.

### Example 101

### General Procedure (B)

### [4-(4-Cyclopropylpiperazin-1-yl)phenyl]phenylmethanone hydrochloride

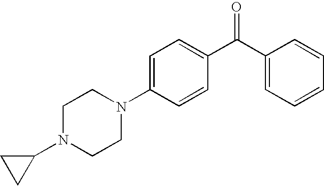

This compound was prepared using the General Procedure (B) from 1-cyclopropylpiperazine and 4-fluorobenzophenone.

¹H NMR (DMSO-d₆) δ0.82 (m, 2H), 1.16 (m, 2H), 2.92 (br s, 1H), 3.30-3.40 (m, 4H), 3.56 (br s, 2H), 4.09 (m, 2H), 7.12 (d, J=8 Hz, 2H), 7.54 (m, 2H), 7.60-7.75 (m, 5H), 10.82 (br s, 1H); HPLC-MS: m/z 307 (MH⁺); Rt=2.00 min.

### Example 102

### General Procedure (B)

### [4-(4-Cyclopropylpiperazin-1-yl)-3,5-difluorophenyl]phenylmethanone hydrochloride

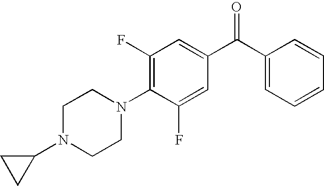

This compound was prepared using the General Procedure (B) from 1-cyclopropylpiperazine and 3,4,5-trifluorobenzophenone.

¹H NMR (DMSO-d₆) δ0.82 (m, 2H), 1.16 (m, 2H), 2.96 (br s, 1H), 3.30-3.75 (m, 8H), 7.43 (d, J=8 Hz, 2H), 7.58 (t, J=8 Hz, 2H), 7.65-7.78 (m, 3H), 10.90 (br s, 1H); HPLC-MS: m/z 343 (MH⁺); Rt=2.29 min.

### Example 103

### General Procedure (B)

### 2-(4-Isopropylpiperazin-1-yl)-5,6,7-trimethoxyquinoline hydrochloride

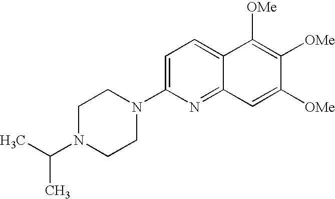

This compound was prepared using the General Procedure (B) from 1-isopropylpiperazine and 2-chloro-5,6,7-trimethoxyquinoline.

¹H NMR (DMSO-d₆) δ1.31 (d, J=7 Hz, 6H), 3.21 (m, 2H), 3.45-3.85 (m, 5H), 3.82 (s, 3H), 3.93 (s, 3H), 3.99 (s, 3H), 4.78 (m, 2H), 7.31 (m, 1H), 7.56 (m, 1H), 8.31 m, 1H), 11.36 (br s, 1H); HPLC-MS: m/z 346 (MH⁺); Rt=1.22 min.

### Example 104

### General Procedure (B)

### 2-(4-Cyclopropylpiperazin-1-yl)-5,6,7-trimethoxyquinoline hydrochloride

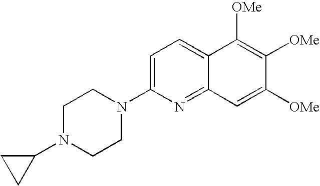

This compound was prepared using the General Procedure (B) from 1-cyclopropylpiperazine and 2-chloro-5,6,7-trimethoxyquinoline.

¹H NMR (DMSO-d₆) δ0.82 (m, 2H), 1.22 (m, 2H), 2.87 (br s, 1H), 3.30-4.10 (m, 6H), 3.83 (s, 3H), 3.93 (s, 3H), 4.00 (s, 3H), 4.76 (m, 2H), 7.33 (m, 1H), 7.65 (br s, 1H), 8.34 (m, 1H), 11.62 (br s, 1H); HPLC-MS: m/z 344 (MH⁺); Rt=1.46 min.

### Example 105

### General Procedure (B)

### 2-(4-Cyclopropylpiperazin-1-yl)-6-trifluoromethylsulfanylquinoline hydrochloride

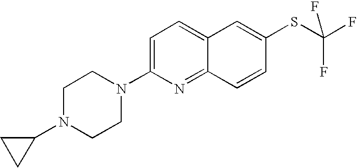

This compound was prepared using the General Procedure (B) from 1-cyclopropylpiperazine and 2-chloro-6-trifluoromethylsulfanylquinoline.

¹H NMR (DMSO-d₆) δ0.82 (m, 2H), 1.20 (m, 2H), 2.86 (br s, 1H), 3.20-3.70 (m, 6H), 4.75 (m, 2H), 7.53 (m, 1H), 7.86 (m, 2H), 8.20-8.40 (m, 2H), 11.28 (br s, 1H); HPLC-MS: m/z 354 (MH⁺); Rt=2.61 min.

### Example 106

### General Procedure (B)

### 7-Chloro-2-(4-cyclopropylpiperazin-1-yl)-6-methoxyquinoline hydrochloride

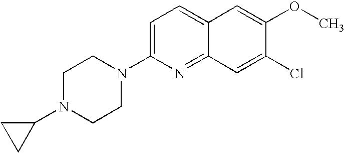

This compound was prepared using the General Procedure (B) from 1-cyclopropylpiperazine and 2,7-dichloro-6-methoxyquinoline.

¹H NMR (DMSO-d₆) δ0.82 (m, 2H), 1.16 (m, 2H), 2.88 (m, 1H), 3.25-3.70 (m, 6H), 3.94 (s, 3H), 4.62 (m, 2H), 7.45 (d, J=8 Hz, 1H), 7.51 (s, 1H), 7.96 (br s, 1H), 8.23 (d, J=8 Hz, 1H), 10.98 (br s, 1H); HPLC-MS: m/z 318 (MH⁺); Rt=1.80 min.

### Example 107

### General Procedure (B)

### 5,7-Dichloro-2-(4-cyclopropylpiperazin-1-yl)quinoline hydrochloride

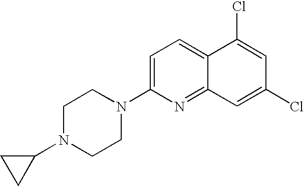

This compound was prepared using the General Procedure (B) from 1-cyclopropylpiperazine and 2,5,7-trichloroquinoline.

¹H NMR (DMSO-d₆) δ0.82 (m, 2H), 1.19 (m, 2H), 2.86 (m, 1H), 3.20-3.65 (m, 6H), 4.69 (m, 2H), 7.51 (d, J=8 Hz, 1H), 7.56 (s, 1H), 7.68 (s, 1H), 8.30 (d, J=8 Hz, 1H), 11.24 (br s, 1H); HPLC-MS: m/z 322 (MH⁺); Rt=2.78 min.

### Example 108

### General Procedure (B)

### 1-Cyclopropyl-4-[5-(4-trifluoromethylphenyl)pyridin-2-yl]piperazine hydrochloride

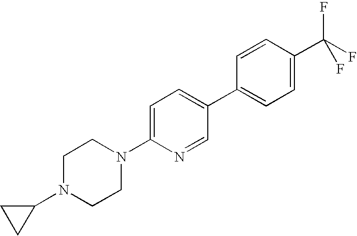

The required 2-chloro-5-(4-trifluorophenyl)pyridine was prepared as described by R. Church, R. Trust, J. D. Albright, and D. Powell, J. Org. Chem. 1995, 60, 3750-3758, in the following way:

To a solution of Vilsmeier reagent prepared from dimethylformamide (5.98 g, 0.082 mol) and phosphorus oxychloride (22.5 g, 0.146 mol) at 10° C., 4-(trifluoromethyl)phenyl acetic acid (6.64 g, 0.033 mol) was added. The mixture was stirred at 70° C. for 8 h. After cooling to ambient temperature, the mixture was added slowly to a mixture of ice and water (temperature was <10° C.) and then the solution of Na₂CO₃ was added slowly until pH 11 was reached. Toluene (125 ml) was added to the alkaline mixture and the resulting mixture was refluxed for 1.5 h. After cooling to ambient temperature the separated water layer was extracted with toluene (100 ml). The combined organic layers were washed with water, dried (Na₂SO₄), and concentrated under reduced pressure.The resulting solid was recrystallized from a mixture of dichloromethane and heptane to yield 6.98 g (87%) of 3-dimethylamino-2-(4-trifluoromethylphenyl)propenal as yellow crystals, mp. 97° C.

¹H NMR spectrum (CDCl₃) δ9.10 (s, 1H), 7.57 (m, 2H), 7.32 (m, 2H), 6.95 (s, 1H), 2.85 (s, 6H); R_{F} (SiO₂, chloroform/methanol 95:5) 0.34.

To a solution of sodium methoxide (3.64 g, 0.068 mol) in methanol (68 ml) cyanoacetamide (6.95 g, 0.082 mol) and the previous product (6.98 g, 0.029 mol) were added. The mixture was stirred at ambient temperature for 1.5 h and then refluxed for 8 h. During this time a yellow solid precipitated. The reaction mixture was diluted with water (75 ml), and acidified with 10% aqueous hydrochloric acid. The yellow solid was filtered off, washed with water, ethanol, diethyl ether, and then with hexane. This afforded 2-hydroxy-5-(4-trifluoromethylphenyl)nicotinonitrile (6.66 g, 87%) as a yellow solid, mp. 235-242° C.

¹H NMR (DMSO-d₆) δ8.42 (m, 1H), 7.91 (m, 1H), 7.66 (m, 4H); R_{F} (SiO₂, chloroform/methanol 95:5) 0.18.

The previous product (6.66 g, 0.025 mol) was added to a mixture of acetic acid (100 ml) and concentrated hydrochloric acid (70 ml). The reaction mixture was refluxed for 18 h, diluted with water (200 ml), and allowed to cool to room temperature while stirring. The solid was filtered off, washed with water and then with 50% aqueous ethanol, to yield 5.42 g (77%) of 2-hydroxy-5-(4-trifluoromethylphenyl)nicotinic acid as a light-gray solid, mp. 305-315° C.

¹H NMR (DMSO-d₆) δ8.71 (d, 1H), 8.43 (d, 1H), 7.96 (m, 2H), 7.81 (m, 2H); R_{F} (SiO₂, chloroform/methanol 95:5): 0.13.

A mixture of the previous product (5.42 g, 0.019 mol) and freshly distilled quinoline (50 ml) was stirred at 215° C. for 12 h. The reaction mixture was cooled to ambient temperature and heptane (250 ml) was added. The solid was filtered off, washed with heptane, and recrystallized from a mixture of dichloromethane and heptane to yield 3.92 g (86%) of 2-hydroxy-5-(4-trifluoromethylphenyl)pyridine, mp. 178-182° C.

¹H NMR (CDCl₃) δ7.79 (dd, 2H), 7.68 (d, 3H), 7.52 (d, 2H), 6.73 (d, 1H); R_{F} (SiO₂, chloroform/methanol 95:5) 0.23.

A mixture of phosphorus oxychloride (27.6 g, 0.18 mol) and the previous product (3.92 g, 0.016 mol) was stirred at 105° C. for 10 h. The excess phosphorus oxychloride was evaporated under reduced pressure, and the residue was stripped once with toluene (75 ml). Water 75 (ml) and dichloromethane (75 ml) were added to the residue, the dichloromethane layer was separated and the aqueous phase was extracted with dichloromethane (75 ml). The combined extracts were washed with water, then with solution of sodium hydrogen carbonate, dried (MgSO₄), and concentrated under reduced pressure, to yield 2.95 g (72%) of 2-chloro-5-(4-trifluoromethylphenyl)pyridine as light-brown crystals, mp 98-101° C.

¹H NMR (CDCl₃) δ8.62 (dd, 1H), 7.86 (dd, 1H), 7.44 (dd, 1H), 7.66 (m, 2H), 7.75 (m, 2H); R_{F} (SiO₂, chloroform/methanol 95:5) 0.94.

This product was treated with 1-cyclopropylpiperazine as described in General Procedure (B) to yield the title compound.

¹H NMR (DMSO-d₆) δ0.83 (m, 2H), 1.13 (m, 2H), 2.85 (m, 1H), 3.25-3.75 (m, 6H), 4.51 (m, 2H), 7.12 (d, J=8 Hz, 1H), 7.79 (d, J=8 Hz, 2H), 7.89 (d, J=8 Hz, 2H), 8.05 (m, 1H), 8.59 (m, 1H), 10.56 (br s, 1H); HPLC-MS: m/z 348 (MH⁺); Rt=2.77 min.

### Example 109

### General Procedure (B)

### 3-(4-Cyclopropylpiperazin-1-yl)-6-(4-trifluoromethylphenyl)pyridazine hydrochloride

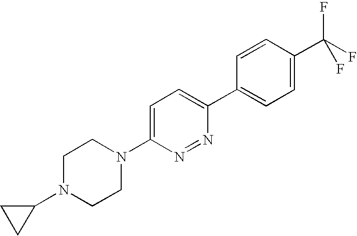

This compound was prepared using the General Procedure (B) from 1-cyclopropylpiperazine and 3-chloro-6-(4-trifluoromethylphenyl)pyridazine.

¹H NMR (DMSO-d₆) δ0.83 (m, 2H), 1.19 (m, 2H), 2.89 (m, 1H), 3.30-3.70 (m, 6H), 4.61 (m, 2H), 7.61 (d, J=8 Hz, 1H), 7.88 (d, J=8 Hz, 2H), 8.22 (d, J=8 Hz, 1H), 8.29 (d, J=8 Hz, 2H), 11.11 (br s, 1H); HPLC-MS: m/z 349 (MH⁺); Rt=2.40 min.

### Example 110

### General Procedure (B)

### 6-(4-Cyclopropylpiperazin-1-yl)-[1,3]dioxolo[4,5-g]quinoline hydrochloride

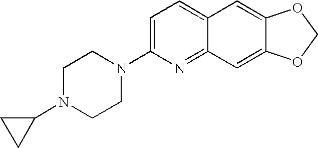

This compound was prepared using the General Procedure (B) from 1-cyclopropylpiperazine and 6-chloro[1,3]dioxolo[4,5-g]quinoline.

¹H NMR (DMSO-d₆) δ0.83 (m, 2H), 1.15 (m, 2H), 2.88 (m, 1H), 3.20-3.70 (m, 6H), 4.59 (m, 2H), 6.17 (s, 2H), 7.29 (m, 3H), 8.13 (m, 1H), 10.80 (br s, 1H); HPLC-MS: m/z 298 (MH⁺); Rt=0.68 min.

### Example 111

### General Procedure (B)

### 6-Cyclohexyl-2-(4-cyclopropylpiperazin-1-yl)quinoline hydrochloride

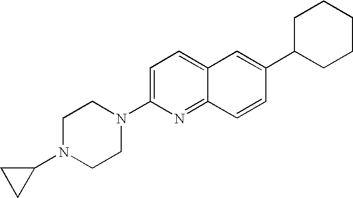

This compound was prepared using the General Procedure (B) from 1-cyclopropylpiperazine and 2-chloro-6-cyclohexylquinoline.

¹H NMR (DMSO-d₆) δ0.83 (m, 2H), 1.10-1.55 (m, 8H), 1.70-1.93 (m, 4H), 2.65 (m, 1H), 2.86 (m, 1H), 3.30-3.70 (m, 6H), 4.72 (m, 2H), 7.50 (m, 1H), 7.66 (m, 1H), 7.72 (m, 1H), 7.99 (br s, 1H), 8.35 (br s, 1H), 11.29 (br s, 1H); HPLC-MS: m/z 336 (MH⁺); Rt=2.55 min.

### Example 112

### General Procedure (B)

### 6-Cyclohexyl-2-(4-isopropylpiperazin-1-yl)quinoline hydrochloride

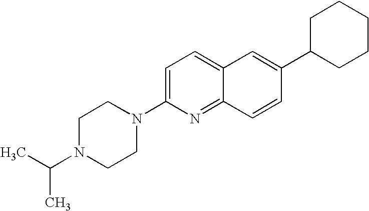

This compound was prepared using the General Procedure (B) from 1-isopropylpiperazine and 2-chloro-6-cyclohexylquinoline.

¹H NMR (DMSO-d₆) δ1.10-1.55 (m, 8H), 1.31 (d, J=7 Hz, 6H), 1.70-1.93 (m, 4H), 2.65 (m, 1H), 3.23 (m, 2H), 3.50-3.85 (m, 3H), 4.80 (m, 2H), 7.51 (m, 1H), 7.67 (m, 1H), 7.73 (m, 1H), 8.08 (br s, 1H), 8.36 (br s, 1H), 11.31 (br s, 1H); HPLC-MS: m/z 338 (MH⁺); Rt=2.50 min.

### Example 113

### General Procedure (B)

### 2-(4-Cyclopropylpiperazin-1-yl)-6,7-dimethoxy-3-methylquinoline hydrochloride

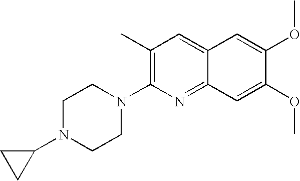

The required 2-chloro-6,7-dimethoxy-3-methylquinoline was prepared according to the procedure published in Tetrahedron Letters 1979, 4885, in the following way:

To a solution of 3,4-dimethoxyaniline (4.70 g, 30.7 mmol) in dichloromethane (50 ml) were added pyridine (8.0 ml, 3 equivalents) and then dropwise propionyl chloride (3.5 ml, 40.5 mmol). After stirring at room temperature for 1 h and 50 min the mixture was poured into a mixture of water (200 ml) and concentrated hydrochloric acid (8 ml). The phases were separated, and the aqueous phase was extracted once with dichloromethane. Washing of the combined organic phases with brine, drying with magnesium sulfate, and concentration yielded 6.89 g of an oil which crystallized after a few minutes. Recrystallization from a mixture of ethyl acetate and heptane yielded 3.60 g (49%) of N-(3,4-dimethoxyphenyl)propionamide as dark crystals.

This anilide (2.1 g, 10.0 mmol) was mixed with DMF (1.1 ml, 15 mmol), and to this mixture POCl₃ (6.5 ml, 70 mmol) was dropwise added at room temperature. When the addition is finished the mixture is stirred at 75° C. for 2 h. The mixture was poured into ice-water (100 ml), stirred for 30 min, and filtered. The solid was stripped with toluene and acetonitrile, to yield 1.60 g (67%) of 2-chloro-6,7-dimethoxy-3-methylquinoline as a solid. This product was treated with 1-cyclopropylpiperazine as described in General Procedure (B) to yield the title compound.

¹H NMR (DMSO-d₆) δ0.83 (m, 2H), 1.23 (m, 2H), 2.42 (s, 3H), 2.94 (m, 1H), 3.40-4.50 (m, 8H), 3.88 (s, 3H), 3.91 (s, 3H), 7.29 (s, 1H), 7.48 (br s, 1H), 8.12 (br s, 1H), 11.24 (br s, 1H); HPLC-MS: m/z 328 (MH⁺); Rt=1.63 min.

### Example 114

### General Procedure (B)

### 6-(4-Isopropylpiperazin-1-yl)-[1,3]dioxolo[4,5-g]quinoline hydrochloride

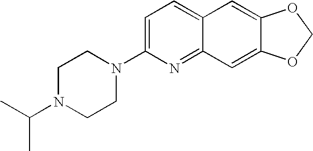

This compound was prepared using the General Procedure (B) from 1-isopropylpiperazine and 6-chloro[1,3]dioxolo[4,5-g]quinoline.

¹H NMR (DMSO-d₆) δ1.31 (d, J=7 Hz, 6H), 3.40 (m, 2H), 3.45-4.00 (m, 5H), 4.72 (m, 2H), 6.21 (s, 2H), 7.34 (m, 2H), 7.62 (br s, 1H), 8.21 (m, 1H), 11.12 (br s, 1H); HPLC-MS: m/z 300 (MH⁺); Rt=0.65 min.

### Example 115

### General Procedure (B)

### [3,5-Difluoro-4-(4-isopropyl-piperazin-1-yl)-phenyl]-piperidin-1-yl-methanone hydrochloride

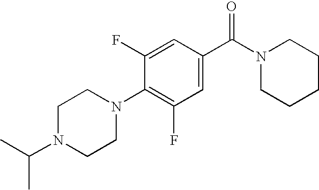

This compound was prepared using the General Procedure (B) by reaction of 1-isopropylpiperazine with 3,4,5-trifluorobenzoic acid piperidide. This reaction yielded two products, namely the current example and example 121.

¹H NMR (DMSO-d₆) δ1.31 (d, J=7 Hz, 6H), 1.40-1.62 (m, 6H), 3.09 (m, 2H), 3.20-3.65 (m, 11H), 7.12 (m, 2H), 10.42 (br s, 1H); HPLC-MS: m/z 352 (MH⁺); Rt=3.75 min.

### Example 116

### General Procedure (B)

### (9a-R)-2-(6,7-Dimethoxyquinolin-2-yl)octahydropyrido[1,2-a]pyrazine hydrochloride

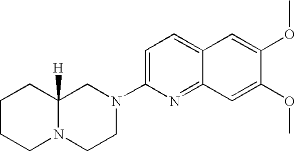

This compound was prepared using the General Procedure (B) from (9a-R)-octahydropyrido[1,2-a]pyrazine and 2-chloro-6,7-dimethoxyquinoline.

¹H NMR (DMSO-d₆) δ1.40-1.55 (m, 1H), 1.65-2.05 (m, 5H), 2.93 (m, 1H), 3.22 (m, 1H), 3.25-3.60 (m, 4H), 3.65-4.20 (m, 1H), 3.87 (s, 3H), 3.90 (s, 3H), 4.79 (m, 2H), 7.40 (m, 2H), 8.00 (br s, 1H), 8.33 (br s, 1H), 11.55 (br s, 1H); HPLC-MS: m/z 328 (MH⁺); Rt=0.97 min.

### Example 117

### General Procedure (B)

### 2-(4-Cyclopropylpiperazin-1-yl)-5,6,7,8-tetrahydroquinoline hydrochloride

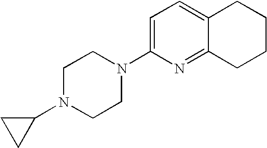

This compound was prepared using the General Procedure (B) from 1-cyclopropylpiperazine and 2-chloro-5,6,7,8-tetrahydroquinoline.

¹H NMR (DMSO-d₆) δ0.81 (m, 2H), 1.17 (m, 2H), 1.67-1.82 (m, 4H), 2.62 (m, 2H), 2.75-2.95 (m, 3H), 3.20-3.65 (m, 6H), 4.37 (m, 2H), 7.02 (br s, 1H), 7.63 (br s, 1H), 11.10 (br s, 1H); HPLC-MS: m/z 258 (MH⁺); Rt=0.62 min.

### Example 118

### General Procedure (B)

### [5-Chloro-6-(4-cyclopropylpiperazin-1-yl)pyridin-3-yl]piperidin-1-ylmethanone hydrochloride

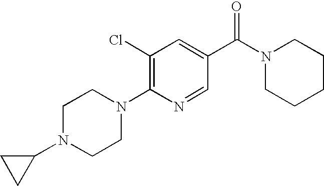

This compound was prepared using the General Procedure (B) from 1-cyclopropylpiperazine and 5,6-dichloronicotinic acid piperidide.

¹H NMR (DMSO-d₆) δ0.82 (m, 2H), 1.12 (m, 2H), 1.45-1.65 (m, 6H), 2.96 (m, 1H), 3.30-3.60 (m, 10H), 3.96 (m, 2H), 7.89 (s, 1H), 8.27 (s, 1H), 10.68 (br s, 1H); HPLC-MS: m/z 349 (MH⁺); Rt=1.50 min.

### Example 119

### General Procedure (B)

### (9a-R)-[6-(Octahydropyrido[1,2-a]pyrazin-2-yl)pyridin-3-yl]piperidin-1-ylmethanone hydrochloride

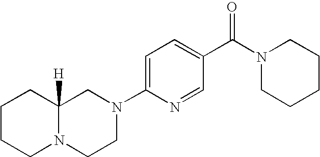

This compound was prepared using the General Procedure (B) from (9a-R)-octahydropyrido[1,2-a]pyrazine and 6-chloronicotinic acid piperidide.

¹H NMR (DMSO-d₆) δ1.51 (m, 3H), 1.60 (m, 2H), 1.78-1.98 (m, 3H), 2.90-3.25 (m, 4H), 3.30-3.50 (m, 5H), 3.74 (m, 6H), 4.50 (m, 2H), 7.02 (d, J=7 Hz, 1H), 7.66 (d, J=7 Hz, 1H), 8.19 (s, 1H), 10.86 (br s, 1H); HPLC-MS: m/z 329 (MH⁺); Rt=1.31 min.

### Example 120

### General Procedure (B)

### [6-(4-Cyclopropylpiperazin-1-yl)pyridin-3-yl]piperidin-1-ylmethanone hydrochloride

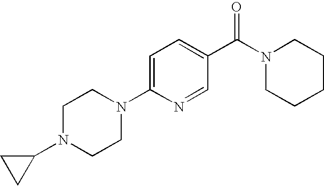

This compound was prepared using the General Procedure (B) from 1-cyclopropylpiperazine and 6-chloronicotinic acid piperidide.

¹H NMR (DMSO-d₆) δ0.81 (m, 2H), 1.17 (m, 2H), 1.51 (m, 4H), 1.60 (m, 2H), 2.87 (m, 1H), 3.20-3.60 (m, 10H), 4.47 (m, 2H), 7.01 (d, J=7 Hz, 1H), 7.67 (d, J=7 Hz, 1H), 8.21 (s, 1H), 11.04 (br s, 1H); HPLC-MS: m/z 315 (MH⁺); Rt=1.22 min.

### Example 121

### General Procedure (B)

### [3,4-Difluoro-5-(4-isopropyl-piperazin-1-yl)-phenyl]-piperidin-1-yl-methanone hydrochloride

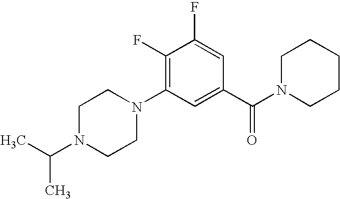

This compound was prepared using the General Procedure (B) by reaction of 1-isopropylpiperazine with 3,4,5-trifluorobenzoic acid piperidide. This reaction yielded two products, namely the current example and example 115

¹H NMR (DMSO-d₆) δ1.31 (d, J=7 Hz, 6H), 1.40-1.65 (m, 6H), 3.12-3.62 (m, 13H), 6.89 (d, J=8 Hz, 1H), 7.09 (m, 1H), 11.09 (br s, 1H); HPLC-MS: m/z 352 (MH⁺); Rt=3.95 min.

### Example 122

### General Procedure (B)

### [4-(4-Isopropyl-piperazin-1-yl)-3-trifluoromethyl-phenyl]-piperidin-1-yl-methanone hydrochloride

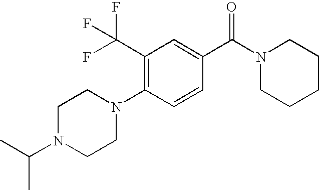

This compound was prepared using the General Procedure (B) by reaction of 1-isopropylpiperazine with 4-fluoro-3-trifluoromethylbenzoic acid piperidide.

¹H NMR (DMSO-d₆) δ1.31 (d, J=7 Hz, 6H), 1.40-1.65 (m, 6H), 3.05 (m, 1H), 3.15 (m, 1H), 3.28 (m, 8H), 3.52 (m, 3H), 7.57 (br d, J=8 Hz, 1H), 7.68 (br s, 1H), 7.71 (br d, J=8 Hz, 1H), 10.39 (br s, 1H); HPLC-MS: m/z 384 (MH⁺); Rt=4.21 min.

### Example 123

### 2-(4-Cyclopropyl-3-methyl-piperazin-1-yl)-6,7-dimethoxy-quinoline hydrochloride

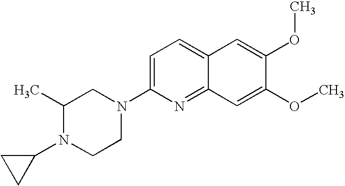

This compound was prepared by reductive cyclopropylation of 6,7-dimethoxy-2-(3-methylpiperazin-1-yl)quinoline as described by Gillaspy, M. L.; Lefker, B. A.; Hada, W. A.; and Hoover, D. J. in Tetrahedron Lett. 1995, 36 (41), 7399-7402.

¹H NMR (DMSO-d₆) δ0.82 (m, 1H), 0.99 (m, 2H), 1.40 (m, 1H), 1.55 (d, J=7 Hz, 3H), 2.76 (m, 1H), 3.30-3.80 (m, 5H), 3.87 (s, 3H), 3.90 (s, 3H), 4.65-4.83 (m, 2H), 7.38 (br s, 2H), 7.82 (br s, 1H), 8.31 (br s, 1H), 11.30 (br s, 1H); HPLC-MS: m/z 328 (MH⁺); Rt=3.09 min.

### Example 124

### General Procedure (B)

### [6-(4-Cyclopropylpiperazin-1-yl)pyridin-3-yl]pyrrolidin-1-yl-methanone hydrochloride

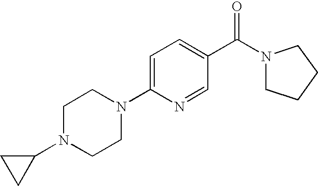

This compound was prepared using the General Procedure (B) from 1-cyclopropylpiperazine and 6-chloronicotinic acid pyrrolidide.

¹H NMR (DMSO-d₆) δ0.81 (m, 2H), 1.15 (m, 2H), 1.84 (m, 4H), 2.88 (m, 1H), 3.15-3.60 (m, 10H), 4.49 (m, 2H), 7.00 (d, J=7 Hz, 1H), 7.83 (d, J=7 Hz, 1H), 8.38 (s, 1H), 10.91 (br s, 1H); HPLC-MS: m/z 301 (MH⁺); Rt=1.22 min.

### Example 125

### General Procedure (B)

### 2-(4-Isopropylpiperazin-1-yl)quinoline-6-carbonitrile hydrochloride

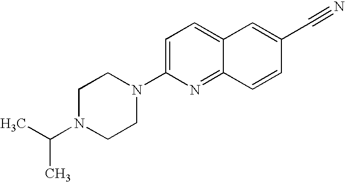

To a mixture of ethyl 3,3-diethoxypropionate (62 g, 326 mmol) and water (100 ml) was added NaOH (16.0 g) while stirring. Stirring at 110° C. (open flask). After 40 min the mixture was homogeneous, heating was interrupted, and the mixture was allowed to cool to room temperature. The mixture was acidified (approx 35 ml conc. HCl, pH 3-2) and extracted (4×dichloromethane). The combined extracts were washed with brine (1×50 ml), dried (magnesium sulfate), and concentrated. 48 g of an oil was obtained.

To the oil was dropwise added thionyl chloride (80 ml). The mixture was stirred at reflux (80° C.) for 1 h 30 min. After careful concentration the residue weighted 48 g (theoretical wheight should be 43 g). The acid chloride was kept overnight at −20° C.

This product is mixed with dichloromethane (70 ml) and 5/7 of this solution (approx 230 mmol) was added to a solution of 4-bromoaniline (34.5 g, 201 mmol) and pyridine (50 ml) in dichloromethane (150 ml), and the mixture was shaken at room temperature over night. The mixture was filtered, and the resulting solid was washed with dichloromethane, and dried, to yield 21 g of N-(4-bromophenyl)-3-ethoxyacrylamide as colorless solid. To the filtrate was added a mixture of water (700 ml) and concentrated hydrochloric acid (50 ml). A solid precipitated upon shaking, and was filtered off, washed with dichloromethane and AcOEt, and dried. Additional 14.4 g of product were obtained.

The filtrates were extracted (3×dichloromethane), washed once with brine, dried, and concentrated. Additional 18 g of product resulted. Total yield: 53.4 g.

This product (58.8 g; 218 mmol) was mixed with ice-cold concentrated sulfuric acid (390 ml) and the mixture was stirred first at 0° C. for 15 min (until almost all acrylamide had dissolved) and then at room temperature for 4 h. The mixture was then poured into ice water (3 l) and allowed to stand overnight. The mixture was filtered, and the solid was washed with water. The solid was transferred into a flask with the aid of acetonitrile, ethanol, and dichloromethane, and the suspension was concentrated under reduced pressure. The residue was resuspended in acetonitrile (300 ml), heated to reflux, and allowed to stand at room temperature overnight. Filtration and drying of the solid under reduced pressure yielded 31.3 g (64%) of 6-bromo-2-quinolone as a yellow solid.

This quinolone (6.28 g, 28.0 mmol) was mixed with copper(I) cyanide (5.02 g, 56.1 mmol) and NMP (15 ml), and the mixture was stirred under reflux (202° C.) for 6 h, and then at room temperature overnight. Water (150 ml) was added, the mixture was filtered, and the solid was washed with water. The solid was resuspended in 1 N hydrochloric acid (200 ml) and iron(III) chloride hexahydrate (17.8 g) was added. The resulting mixture was stirred at room temperature for 3 d, filtered, and the solid was washed once with water, stripped with ethanol, and dried under reduced pressure, to yield 4.33 g (91%) of 6-cyano-2-quinolone as a gray solid. Treatment of the product with POCl₃ and then with 1-isopropylpiperazine as described in the General Procedure (B) yielded the title compound.

¹H NMR (DMSO-d₆) δ1.31 (d, J=7 Hz, 6H), 3.10 (m, 2H), 3.52 (m, 5H), 4.79 (m, 2H), 7.49 (d, J=8 Hz, 1H), 7.71 (d, J=8 Hz, 1H), 7.86 (d, J=8 Hz, 1H), 8.23 (d, J=8 Hz, 1H), 8.35 (s, 1H), 10.73 (br s, 1H); HPLC-MS: m/z 281 (MH⁺); Rt=1.62 min.

### Example 126

### General Procedure (B)

### 3-(4-Cyclopropylpiperazin-1-yl)-6-phenylpyridazine hydrochloride

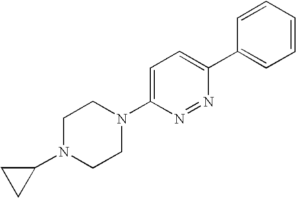

This compound was prepared using the General Procedure (B) from 1-cyclopropylpiperazine and 3-chloro-6-phenylpyridazine.

¹H NMR (DMSO-d₆) δ0.82 (m, 2H), 1.22 (m, 2H), 2.88 (br s, 1H), 3.37 (m, 2H), 3.59 (m, 4H), 4.57 (m, 2H), 7.53 (m, 3H), 7.80 (d, J=8 Hz, 1H), 8.06 (m, 2H), 8.29 (d, J=8 Hz, 1H), 11.47 (br s, 1H); HPLC-MS: m/z 281 (MH⁺); Rt=1.43 min.

### Example 127

### General Procedure (B)

### 3-(4-Cyclopropylpiperazin-1-yl)-6-(3,4-dimethoxyphenyl)pyridazine hydrochloride

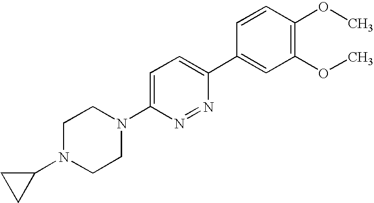

This compound was prepared using the General Procedure (B) from 1-cyclopropylpiperazine and 3-chloro-6-(3,4-dimethoxyphenyl)pyridazine.

¹H NMR (DMSO-d₆) δ0.83 (m, 2H), 1.22 (m, 2H), 2.88 (br s, 1H), 3.36 (m, 2H), 3.60 (m, 4H), 3.84 (s, 3H), 3.87 (s, 3H), 4.53 (m, 2H), 7.14 (d, J=8 Hz, 1H), 7.66 (m, 2H), 7.86 (d, J=8 Hz, 1H), 8.37 (d, J=8 Hz, 1H), 11.43 (br s, 1H); HPLC-MS: m/z 341 (MH⁺); Rt=1.45 min.

### Example 128

### General Procedure (B)

### 7-(4-Isopropylpiperazin-1-yl)-2,3-dihydro-[1,4]dioxino[2,3-g]quinoline

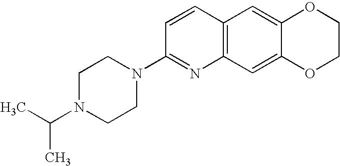

This compound was prepared using the General Procedure (B) from 1-isopropylpiperazine and 7-chloro-2,3-dihydro[1,4,]dioxino[2,3-g]quinoline.

¹H NMR (DMSO-d₆) δ1.00 (d, J=7 Hz, 6H), 2.67 (hept, J=7 Hz, 1H), 3.33 (s, 4H), 3.56 (m, 4H), 4.29 (m, 4H), 6.95 (s, 1H), 7.02 (d, J=8 Hz, 1H), 7.13 (s, 1H), 7.84 (d, J=8 Hz, 1H); HPLC-MS: m/z 314 (MH⁺); Rt=1.14 min.

### Example 129

### 2-(4-Cyclopropyl-3-methylpiperazin-1-yl)quinoline hydrochloride

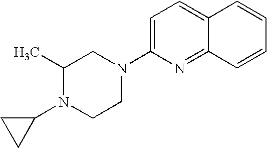

This compound was prepared by reductive cyclopropylation of 2-(3-methylpiperazin-1-yl)quinoline as described by Gillaspy, M. L.; Lefker, B. A.; Hada, W. A.; and Hoover, D. J. in Tetrahedron Lett 1995, 36 (41), 7399-7402.

¹H NMR (DMSO-d₆) δ0.82 (m, 1H), 1.00 (m, 2H), 1.41 (m, 1H), 1.56 (d, J=7 Hz, 3H), 2.76 (m, 1H), 3.25-3.80 (m, 5H), 4.81 (m, 2H), 7.44 (br s, 1H), 7.55 (m, 1H), 7.72 (m, 1H), 7.88 (m, 1H), 8.11 (br s, 1H), 8.37 (br s, 1H), 11.40 (br s, 1H); HPLC-MS: m/z 268 (MH⁺); Rt=1.18 min.

### Example 130

### General Procedure (B)

### 6-Cyclopropylmethoxy-2-(4-cyclopropylpiperazin-1-yl)quinoline hydrochloride

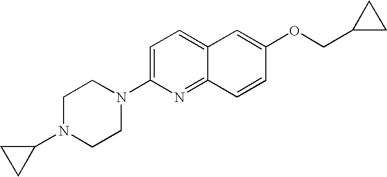

This compound was prepared using the General Procedure (B) from 1-isopropylpiperazine and 2-chloro-6-(cyclopropylmethyloxy)quinoline. The required 2-chloro-6-(cyclopropylmethyloxy)quinoline was prepared by treatment of 6-(cyclopropylmethyloxy)-2-quinolone with POCl₃. 6-(Cyclopropylmethyloxy)-2-quinolone was prepared from the corresponding 6-hydroxyquinolone by treatment with (bromomethyl)cycylopropane and potassium carbonate in dimethyl formamide in the presence of catalytic amounts of sodium iodide.

¹H NMR (DMSO-d₆) δ0.35 (m, 2H), 0.60 (m, 2H), 0.82 (m, 2H), 1.20 (m, 2H), 1.28 (m, 1H), 2.87 (br s, 1H), 3.25-4.20 (m, 6H), 3.92 (d, J=7 Hz, 2H), 4.70 (m, 2H), 7.30-7.60 (m, 3H), 8.08 (br s, 1H), 8.32 (br s, 1H), 11.37 (br s, 1H); HPLC-MS: m/z 324 (MH⁺); Rt=1.94 min.

### Example 131

### General Procedure (B)

### 2-(4-Isopropylpiperazin-1-yl)-6-pyrazol-1-ylquinoline hydrochloride

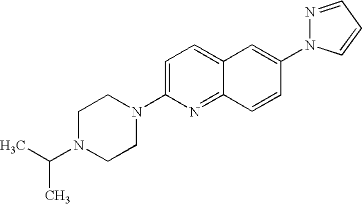

This compound was prepared using the General Procedure (B) from 1-isopropylpiperazine and 2-chloro-6-(1-pyrazolyl)quinoline. The required 2-chloro-6-(1-pyrazolyl)quinoline was prepared by treatment of 6-(1-pyrazolyl)-2-quinolone with POCl₃. 6-(1-Pyrazolyl)-2-quinolone was prepared in the following way:

A mixture of 6-bromo-2-quinolone (3.58 g, 16.0 mmol), DMF (15 ml), pyrazole (1.66 g, 24.4 mmol), potassium carbonate (3.33 g, 24.1 mmol), and copper(I) iodide (0.76 g, 3.99 mmol) was stirred at 160° C. for 22 h. Water (300 ml) was added, and after thorough trituration the mixture was filtered, and the solid was washed with water. The solid was stripped with EtOH, resuspended in a mixture of acetonitrile and ethanol (100 ml, 1:1), heated to reflux, and kept at room temperature overnight. Filtration, washing with a bit of acetonitrile, and drying under reduced pressure yielded 1.7 g (50%) of 6-(1-pyrazolyl)-2-quinolone as a metallic-green solid.

¹H NMR (DMSO-d₆) δ1.32 (d, J=7 Hz, 6H), 3.10-3.70 (m, 7H), 4.81 (m, 2H), 6.60 (m, 1H), 7.56 (d, J=8 Hz, 1H), 7.81 (s, 1H), 8.09 (m, 1H), 8.22 (d, J=8 Hz, 1H), 8.32 (s, 1H), 8.38 (d, J=8 Hz, 1H), 8.58 (m, 1H), 11.11 (br s, 1H); HPLC-MS: m/z 322 (MH⁺); Rt=1.67 min.

### Example 132

### General Procedure (B)

### 2-(4-Isopropylpiperazin-1-yl)quinolin-6-ol hydrochloride

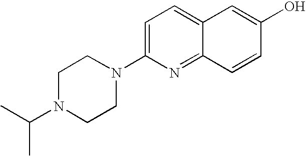

This compound was prepared using the General Procedure (B) from 1-isopropylpiperazine and 2-chloro-6-hydroxyquinoline.

¹H NMR (DMSO-d₆) δ1.31 (d, J=7 Hz, 6H), 3.15-3.90 (m, 7H), 4.79 (m, 2H), 7.22 (m, 1H), 7.33 (d, J=8 Hz, 1H), 7.53 (d, J=8 Hz, 1H), 8.13 (br s, 1H), 8.35 (d, J=8 Hz, 1H), 10.17 (br s, 1H), 11.41 (br s, 1H); HPLC-MS: m/z 272 (MH⁺); Rt=0.40 min.

### Example 133

### General Procedure (B)

### 2-(4-Cyclopropylpiperazin-1-yl)quinoline-6-carbonitrile hydrochloride

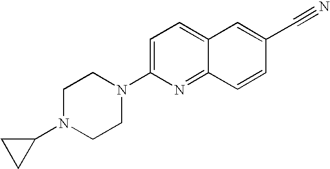

This compound was prepared using the General Procedure (B) from 1-cyclopropylpiperazine and 2-chloro-6-cyanoquinoline.

¹H NMR (DMSO-d₆) δ0.83 (m, 2H), 1.15 (m, 2H), 2.87 (br s, 1H), 3.20-3.65 (m, 6H), 4.74 (m, 2H), 7.49 (d, J=8 Hz, 1H), 7.72 (d, J=8 Hz, 1H), 7.86 (d, J=8 Hz, 1H), 8.24 (d, J=8 Hz, 1H), 8.35 (s, 1H), 10.88 (br s, 1H); HPLC-MS: m/z 279 (MH⁺); Rt=1.43 min.

### Example 134

### General Procedure (B)

### (9a-R)-2-(Octahydropyrido[1,2-a]pyrazin-2-yl)quinoline-6-carbonitrile hydrochloride

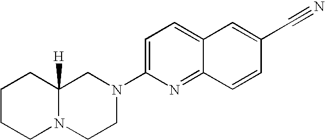

This compound was prepared using the General Procedure (B) from (9a-R)-octahydropyrido[1,2-a]pyrazine and 2-chloro-6-cyanoquinoline.

¹H NMR (DMSO-d₆) δ1.40-1.60 (m, 1H), 1.65-2.00 (m, 5H), 2.85-3.65 (m, 7H), 4.80 (m, 2H), 7.53 (d, J=8 Hz, 1H), 7.79 (d, J=8 Hz, 1H), 7.89 (d, J=8 Hz, 1H), 8.26 (d, J=8 Hz, 1H), 8.37 (s, 1H), 11.31 (br s, 1H); HPLC-MS: m/z 293 (MH⁺); Rt=1.72 min.

### Example 135

### General Procedure (B)

### 4-Chloro-6-(4-isopropylpiperazin-1-yl)-3-phenylpyridazine hydrochloride

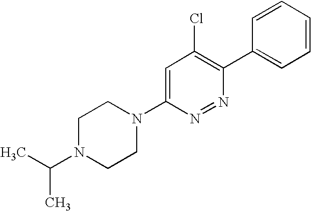

This compound was prepared using the General Procedure (B) from 1-isopropylpiperazine and 4,6-dichloro-3-phenylpyridazine.

¹H NMR (DMSO-d₆) δ1.31 (d, J=7 Hz, 6H), 3.13 (m, 2H), 3.40-3.65 (m, 5H), 4.63 (m, 2H), 7.50 (m, 3H), 7.63 (m, 2H), 7.78 (s, 1H), 11.01 (br s, 1H); HPLC-MS: m/z 317 (MH⁺); Rt=2.11 min.

### Example 136

### General Procedure (B)

### 3-(4-Cyclopropylpiperazin-1-yl)-6-(3-trifluoromethylphenyl)pyridazine hydrochloride

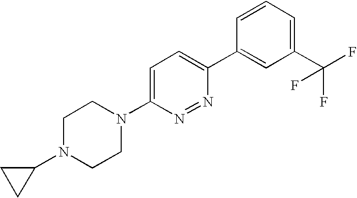

This compound was prepared using the General Procedure (B) from 1-cyclopropylpiperazine and 3-chloro-6-(3-trifluoromethylphenyl)pyridazine.

¹H NMR (DMSO-d₆) δ0.83 (m, 2H), 1.22 (m, 2H), 2.89 (br s, 1H), 3.35 (m, 2H), 3.59 (m, 4H), 4.63 (m, 2H), 7.68 (d, J=8 Hz, 1H), 7.77 (m, 1H), 7.84 (m, 1H), 8.32 (d, J=8 Hz, 1H), 8.40 (m, 2H), 11.40 (br s, 1H); HPLC-MS: m/z 349 (MH⁺); Rt=2.43 min.

### Example 137

### 1-[2-(4-Isopropylpiperazin-1-yl)quinolin-6-yl]ethanone hydrochloride

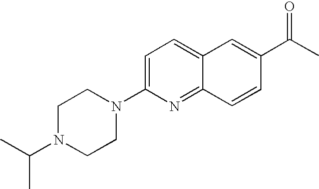

This product was prepared by treatment of 2-(4-isopropylpiperazin-1-yl)quinoline-6-carbonitrile with an excess of methylmagnesium bromide in tetrahydrofuran.

¹H NMR (DMSO-d₆) δ1.31 (d, J=7 Hz, 6H), 2.65 (s, 3H), 3.18 (m, 2H), 3.40-3.80 (m, 5H), 4.82 (m, 2H), 7.52 (d, J=8 Hz, 1H), 7.91 (br s, 1H), 8.15 (d, J=8 Hz, 1H), 8.40 (d, J=8 Hz, 1H), 8.54 (s, 1H), 11.16 (br s, 1H); HPLC-MS: m/z 298 (MH⁺); Rt=1.47 min.

### Example 138

### General Procedure (B)

### 3-(4-Isopropylpiperazin-1-yl)-6-phenylpyridazine-4-carbonitrile hydrochloride

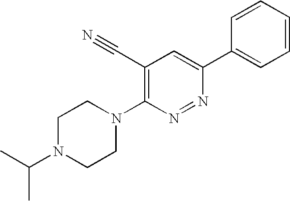

This compound was prepared using the General Procedure (B) from 1-isopropylpiperazine and 3-chloro-6-phenylpyridazine-4-carbonitrile.

¹H NMR (DMSO-d₆) δ1.32 (d, J=7 Hz, 6H), 3.23 (m, 2H), 3.50-3.75 (m, 5H), 4.46 (m, 2H), 7.55 (m, 3H), 8.13 (d, J=7 Hz, 2H), 8.72 (s, 1H), 10.67 (br s, 1H); HPLC-MS: m/z 308 (MH⁺); Rt=2.16 min.

### Example 139

### General Procedure (B)

### 6-Bromo-2-(4-isopropylpiperazin-1-yl)quinoline

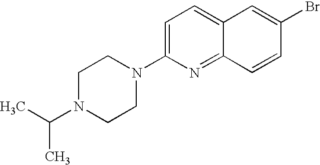

This compound was prepared using the General Procedure (B) from 1-isopropylpiperazine and 6-bromo-2-chloroquinoline.

¹H NMR (DMSO-d₆) δ1.00 (d, J=7 Hz, 6H), 2.52 (m, 4H), 2.69 (sept, J=7 Hz, 1H), 3.68 (m, 4H), 7.28 (d, J=8 Hz, 1H), 7.48 (d, J=8 Hz, 1H), 7.60 (dd, J=1 Hz, 8 Hz, 1H), 7.93 (d, J=1 Hz, 1H), 8.00 (d, J=8 Hz, 1H).

### Example 140

### General Procedure (B)

### 2-(4-Cyclopropylpiperazin-1-yl)-6-pyrazol-1-ylquinoline hydrochloride

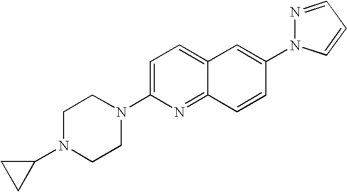

This compound was prepared using the General Procedure (B) from 1-cyclopropylpiperazine and 2-chloro-6-pyrazol-1-ylquinoline.

¹H NMR (DMSO-d₆) δ0.83 (m, 2H), 1.22 (m, 2H), 2.88 (m, 1H), 3.30-3.80 (m, 6H), 4.79 (m, 2H), 6.61 (m, 1H), 7.59 (d, J=6 Hz, 1H), 7.81 (s, 1H), 8.10-8.30 (m, 2H), 8.34 (s, 1H), 8.41 (d, J=6 Hz, 1H), 8.59 (s, 1H), 11.47 (br s, 1H); HPLC-MS: m/z 320 (MH⁺); Rt=1.64 min.

### Example 141

### General Procedure (B)

### 7-Chloro-2-(4-cyclopropylpiperazin-1-yl)quinolin-6-ol hydrochloride

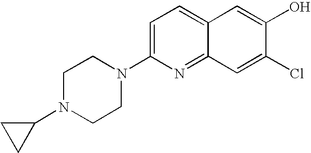

This compound was prepared using the General Procedure (B) from 1-cyclopropylpiperazine and 2,7-dichloro-6-hydroxyquinoline. The required 2,7-dichloro-6-hydroxyquinoline was prepared by demethylation with boron tribromide in dichloromethane of 2,7-dichloro-6-methoxyquinoline, which was prepared from 3-chloro-4-methoxyaniline as described by F. Effenberger and W. Hartmann in Chemische Berichte 1969, 102, 3260-3267.

¹H NMR (DMSO-d₆) δ0.83 (m, 2H), 1.14 (m, 2H), 2.88 (m, 1H), 3.20-3.80 (m, 6H), 4.61 (m, 2H), 7.32 (s, 1H), 7.42 (m, 1H), 7.96 (m, 1H), 8.21 (m, 1H), 10.65 (br s, 1H), 10.84 (br s, 1H); HPLC-MS: m/z 304 (MH⁺); Rt=1.06 min.

### Example 142

### [2-(4-Cyclopropylpiperazin-1-yl)quinolin-6-yl]-(4-fluorophenyl)methanone hydrochloride

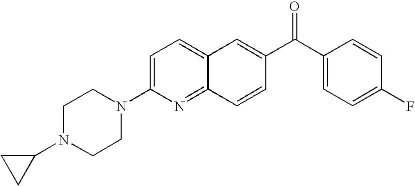

This product was prepared by treatment of 2-(4-cyclopropylpiperazin-1-yl)quinoline-6-carbonitrile with an excess of (4-fluorophenyl)magnesium bromide in tetrahydrofuran.

¹H NMR (DMSO-d₆) δ0.84 (m, 2H), 1.14 (m, 2H), 2.89 (m, 1H), 3.25-3.90 (m, 6H), 4.77 (m, 2H), 7.44 (m, 3H), 7.76 (m, 1H), 7.86 (m, 2H), 7.98 (m, 1H), 8.21 (s, 1H), 8.34 (d, J=8 Hz, 1H), 10.65 (br s, 1H); HPLC-MS: m/z 376 (MH⁺); Rt=2.61 min.

### Example 143

### General Procedure (B)

### Cyclopropyl-[2-(4-cyclopropylpiperazin-1-yl)quinolin-6-yl]methanone hydrochloride

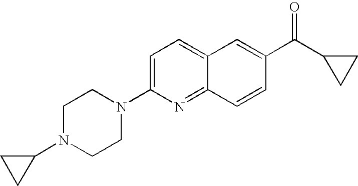

This product was prepared by treatment of 2-(4-cyclopropylpiperazin-1-yl)quinoline-6-carbonitrile with an excess of cyclopropylmagnesium bromide in tetrahydrofuran.

¹H NMR (DMSO-d₆) δ0.83 (m, 2H), 1.07 (m, 4H), 1.18 (m, 2H), 2.88 (m, 1H), 3.00 (quint, J=7 Hz, 1H), 3.30-3.70 (m, 6H), 4.76 (m, 2H), 7.50 (d, J=8 Hz, 1H), 7.82 (br s, 1H), 8.18 (d, J=8 Hz, 1H), 8.37 (d, J=8 Hz, 1H), 8.66 (s, 1H), 11.05 (br s, 1H); HPLC-MS: m/z 322 (MH⁺); Rt=1.98 min.

### Example 144

### General Procedure (B)

### 2-(4-Cyclopropylpiperazin-1-yl)-6-(4-fluorobenzyloxy)quinoline hydrochloride

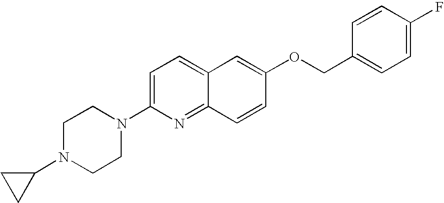

This compound was prepared using the General Procedure (B) from 1-cyclopropylpiperazine and 2-chloro-6-(4-fluorobenzyloxy)quinoline.

¹H NMR (DMSO-d₆) δ0.83 (m, 2H), 1.20 (m, 2H), 2.87 (m, 1H), 3.30-3.80 (m, 6H), 4.70 (m, 2H), 5.18 (s, 2H), 7.25 (t, J=8 Hz, 2H), 7.54 (m, 5H), 8.08 (br s, 1H), 8.33 (m, 1H), 11.34 (br s, 1H); HPLC-MS: m/z 378 (MH⁺); Rt=2.53 min.

### Example 145

### 6-Cyclohex-1-enyl-2-(4-isopropylpiperazin-1-yl)quinoline hydrochloride

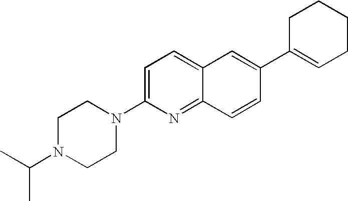

This product was prepared by treatment of 6-bromo-2-(4-isopropylpiperazin-1-yl)quinoline with butyllithium and then with cyclohexanone. Upon acidic workup the tertiary alcohol underwent elimination to yield the title compound.

¹H NMR (DMSO-d₆) δ1.31 (d, J=7 Hz, 6H), 1.60-1.80 (m, 4H), 2.22 (m, 2H), 2.45 (m, 2H), 3.23 (m, 2H), 3.45-4.00 (m, 5H), 4.78 (m, 2H), 6.34 (m, 1H), 7.50 (m, 1H), 7.86 (m, 2H), 7.98 (br s, 1H), 8.34 (m, 1H), 11.06 (br s, 1H); HPLC-MS: m/z 336 (MH⁺); Rt=2.50 min.

### Example 146

### General Procedure (C)

### 1-(Biphenyl-3-yl)-4-(cyclopentyl)piperazine

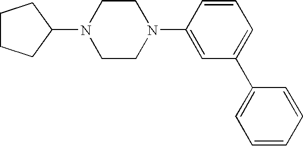

A mixture of 3-bromobiphenyl (300 mg, 1.28 mmol), 1-cyclopentylpiperazine (238 mg, 1.54 mmol), sodium tert.-butoxide (173 mg, 1.8 mmol), tris(dibenzylideneacetone)dipalladium (12 mg, 0.01 mmol) and racemic 2,2′-bis(diphenylphosphino)-1,1′binaphthyl (24 mg, 0.04 mmol) in toluene (11 ml) was mixed under nitrogen in a closed reaction vessel. The reaction mixture was stirred at 80° C. for 3 days in a closed reaction vessel. It was cooled to room temperature and washed with water (2×10 ml). The combined organic layers were extracted with 1 N hydrochloric acid (2×20 ml). The combined aqueous extracts were made basic with an 1 N aqueous sodium hydroxide solution and extracted with tert-butyl methyl ether (3×20 ml). The tert-butyl methyl ether layers were dried over magnesium sulphate. The solvent was removed in vacuo to give 220 mg of the title compound.

¹H-NMR (CDCl₃, two sets of signals, broad signals) δ1.35-1.85 (m, 6 H); 1.95 (m, 2 H); 2.55 (m, 1 H); 2.70 (m, 4 H); 3.30 (m, 4 H); 6.95 (d, 1 H); 7.05 (d, 1 H); 7.15 (s, 1 H); 7.35 (t, 2 H); 7.45 (t, 2 H); 7.60 (d, 2 H). HPLC method B: elution at 10.45 min.

The title compound was transferred into its hydrochloride salt, by dissolving it in ethyl acetate (5 ml). A 3.2 M solution of hydrogen chloride in ethyl acetate (5 ml) was added. The solvent was removed in vacuo. The residue was dissolved in ethanol (50 ml). The solvent was removed in vacuo.

### Example 147

### General Procedure (C)

### 1-Cyclopentyl-4-[4-(2-(pyrrolidin-1-yl)ethoxy)phenyl]piperazine

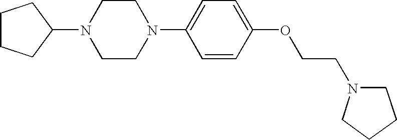

86 mg of the title compound were synthesized as described for 1-(biphenyl-3-yl)-4-(cyclopentyl)piperazine, using 1-(2-(4-bromophenoxy)ethyl)pyrrolidine instead of 3-bromobiphenyl.

¹H-NMR (DMSO-d₆) δ1.55 (m, 2 H); 1.75 (m, 2 H); 1.85 (m, 4 H); 2.00 (m, 4 H); 3.10 (m, 6 H); 3.40-3.70 (m, 9 H); 4.30 (t, 2 H); 7.00 (AB, 4 H); 10.80 (br, 1 H); 11.10 (br, 1 H). HPLC method C: elution at 2.24 min. MS: calc. for [M+H]⁺: 344; found: 344.

The title compound was transferred into its hydrochloride salt, by dissolving it in ethyl acetate (5 ml). A 3.2 M solution of hydrogen chloride in ethyl acetate (5 ml) was added. The solvent was removed in vacuo. The residue was dissolved in ethanol (50 ml). The solvent was removed in vacuo.

### Example 148

### General Procedure (C)

### 1-(Biphenyl-4-yl)-4-(cyclopentyl)piperazine

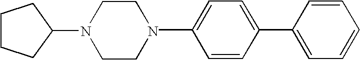

180 mg of the title compound were synthesized as described for 1-(biphenyl-3-yl)-4-(cyclopentyl)piperazine, using 4-bromobiphenyl instead of 3-bromobiphenyl.

¹H-NMR (CDCl₃) δ1.80-2.00 (m, 8 H); 2.55 (m, 1 H); 2.70 (m, 4 H); 3.20 (m, 4 H); 7.00 (d, 2 H); 7.30 (m, 1 H); 7.40 (t, 2 H); and 7.55 (m, together 4 H). HPLC method C: elution at 4.52 min.

The title compound was transferred into its hydrochloride salt, by dissolving it in ethyl acetate (5 ml). A 3.2 M solution of hydrogen chloride in ethyl acetate (5 ml) was added. The solvent was removed in vacuo. The residue was dissolved in ethanol (50 ml). The solvent was removed in vacuo.

### Example 149

### General Procedure (C)

### [3-(4-(Cyclopentyl)piperazin-1-yl)phenyl]-(4-fluorophenyl)methanone

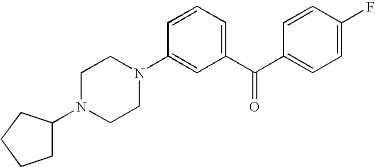

300 mg of the title compound were synthesized as described for 1-(biphenyl-3-yl)-4-(cyclopentyl)piperazine, using 3-bromo-4′-fluorobenzophenone instead of 3-bromobiphenyl.

¹H-NMR (CDCl₃) δ1.45 (m, 2 H); 1.60 (m, 2 H); 1.75 (m, 2 H); 1.90 (m, 2 H); 2.55 (quintet, 1 H); 2.70 (m, 4 H); 3.30 (m, 4 H); 7.15 (m, 4 H); 7.35 (m, 2 H); 7.85 (m, 2 H). HPLC method C: elution at 4.22 min. MS: calc. for [M+H]⁺: 353; found: 353.

The title compound was transferred into its hydrochloride salt, by dissolving it in ethyl acetate (5 ml). A 3.2 M solution of hydrogen chloride in ethyl acetate (5 ml) was added. The solvent was removed in vacuo. The residue was dissolved in ethanol (50 ml). The solvent was removed in vacuo.

The following compounds are also within the scope of the present invention:

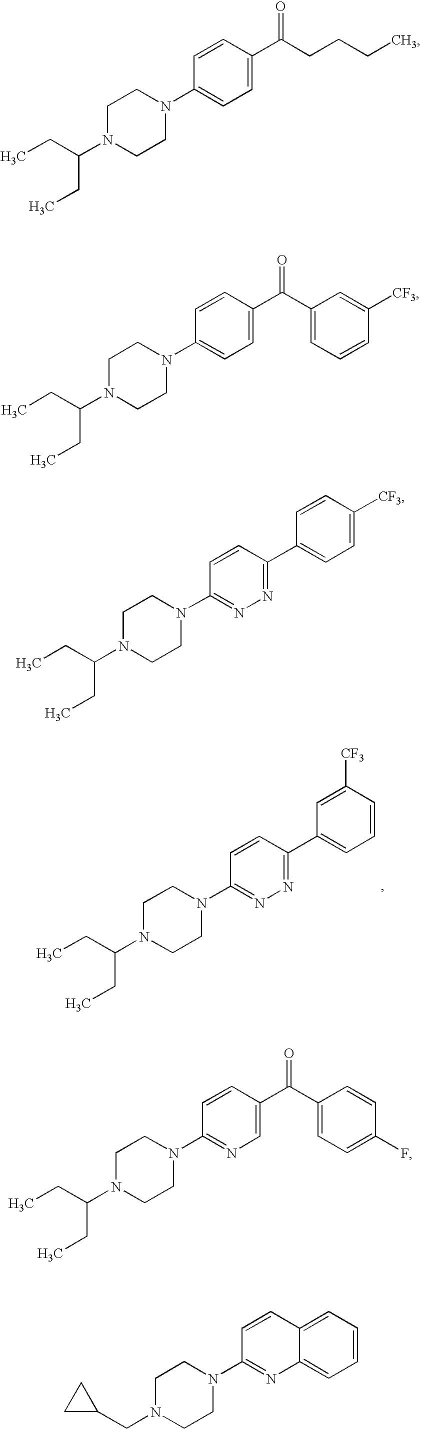

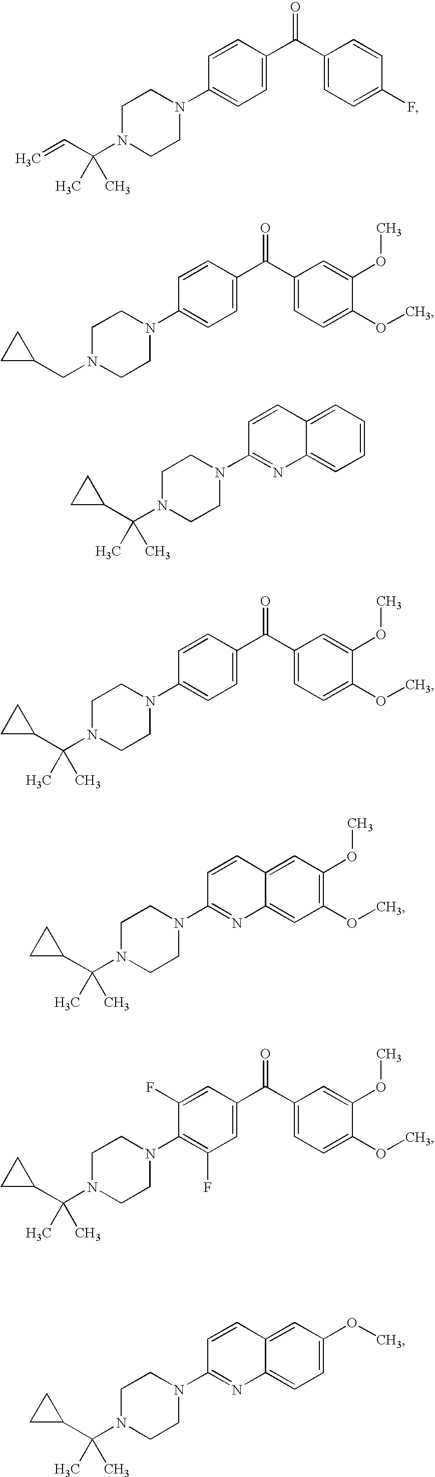

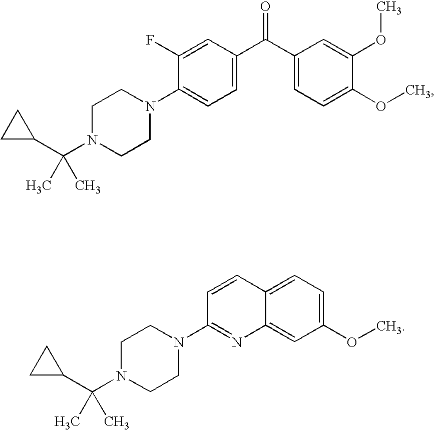

Pharmacological Methods

The ability of the compounds to interact with the histamine H3 receptor can be determined by the following in vitro binding assays.

Binding Assay I

Rat cerebral cortex is homogenized in ice cold K-Hepes, 5 mM MgCl₂ pH 7.1 buffer. After two differential centrifugations the last pellet is resuspended in fresh Hepes buffer containing 1 mg/ml bacitracin. Aliquots of the membrane suspension (400 μg/ml) are incubated for 60 min at 25° C. with 30 pM [¹²⁵I]-iodoproxifan, a known histamine H3 receptor antagonist, and the test compound at various concentrations. The incubation is stopped by dilution with ice-cold medium, followed by rapid filtration through Whatman GF/B filters pretreated for 1 hour with 0.5% polyethyleneimine. The radioactivity retained on the filters is counted using a Cobra II auto gamma counter. The radioactivity of the filters is indirectly proportional to the binding affinity of the tested compound. The results are analyzed by nonlinear regression analysis.

Binding Assay II

The H3-receptor agonist ligand R-(α-methyl[³H]histamine (RAMHA) is incubated with isolated rat cortex cell-membranes at 25° C. for 1 hour, followed by a filtration of the incubate through Whatman GF/B filters. Radioactivity retained on the filters is measured using a beta counter. Male Wistar rats (150-200 g) are decapitated and cerebral cortex is quickly dissected out and frozen immediately on dry ice. Tissue is kept at −80° C. until membrane preparation. During the membrane preparation the tissue is kept on ice all the time. Rat cerebral cortex is homogenized in 10 volumes (w/w) ice-cold Hepes buffer (20 mM Hepes, 5 mM MgCl₂ pH 7.1 (KOH)+1 mg/ml bacitracin) using an Ultra-Turrax homogenizer for 30 seconds. The homogenate is centrifuged at 140 g in 10 min. The supernatant is transferred to a new test tube and centrifuged for 30 min at 23 000 g. Pellet is resuspended in 5-10 ml Hepes buffer, homogenized and centrifuged for 10 min at 23 000 g. This short centrifugation step is repeated twice. After the last centrifugation the pellet is resuspended in 2-4 ml Hepes buffer and the protein concentration is determined. The membranes are diluted to a protein concentration of 5 mg/ml using Hepes buffer, aliquoted and stored at −80° C. until use.

50 μl test-compound, 100 μl membrane (200 μg/ml), 300 μl Hepes buffer and 50 μl R-α-methyl[³H]histamine (1 nM) are mixed in a test tube. The compounds to be tested are dissolved in DMSO and further diluted in H₂O to the desired concentrations. Radioligand and membranes are diluted in Hepes buffer+1 mg/ml bacitracin. The mixture is incubated for 60 min at 25° C. Incubation is terminated by adding 5 ml ice-cold 0.9% NaCl, followed by rapid filtration through Whatman GF/B filters pre-treated for 1 hour with 0.5% polyethyleneimine. The filters are washed with 2×5 ml ice-cold NaCl. To each filter a 3 ml scintillation cocktail is added and the radioactivity retained is measured with a Packard Tri-Carb beta counter. IC₅₀ values are calculated by non-linear regression analysis of binding curves (6 points minimum) using the windows program GraphPad Prism, GraphPad software, USA.

Binding Assay III

The human H3 receptor is cloned by PCR and subcloned into the pcDNA3 expression vector. Cells stably expressing the H3 receptor are generated by transfecting the H3-expression vectors into HEK 293 cells and using G418 to select for H3 clones. The human H3-HEK 293 clones are cultured in DMEM (GIBCO-BRL) with glutamax, 10% foetal calf serum, 1% penicillin/streptavidin and 1 mg/ml G 418 at 37° C. and 5% CO₂. Before harvesting, the confluent cells are rinsed with PBS and incubated with Versene (proteinase, GIBCO-BRL) for approximately 5 min. The cells are flushed with PBS and DMEM and the cell suspension collected in a tube and centrifuged for 5-10 min at 1500 rpm in a Heraeus Sepatech Megafuge 1.0. The pellet is resuspended in 10-20 vol. Hepes buffer (20 mM Hepes, 5 mM MgCl₂, pH 7.1 (KOH)) and homogenized for 10-20 seconds using an Ultra-Turrax homogenizer. The homogenate is centrifuged for 30 min at 23 000 g. The pellet is resuspended in 5-10 ml Hepes buffer, homogenized 5-10 seconds with the Ultra-Turrax and centrifuged for 10 min at 23 000 g. Following this centrifugation step, the membrane pellet is resuspended in 2-4 ml Hepes buffer, homogenized with a syringe or Teflon homogenizer, and the protein concentration determined. The membranes are diluted to a protein concentration of 1-5 mg/ml in Hepes buffer, aliquoted and kept at −80° C. until use.

Aliquots of the membrane suspension are incubated for 60 min at 25° C. with 30 pM [¹²⁵I]-iodoproxifan, a known compound with high affinity for the H3 receptor, and the test compound at various concentrations. The incubation is stopped by dilution with ice-cold medium, followed by rapid filtration through Whatman GF/B filters pretreated for 1 hour with 0.5% polyethyleneimine. The radioactivity retained on the filters is counted using a Cobra II auto gamma counter. The radioactivity of the filters is indirectly proportional to the binding affinity of the tested compound. The results are analysed by nonlinear regression analysis.

When tested, the present compounds of the formula (I) generally show a high binding affinity to the histamine H3 receptor.

Preferably, the compounds according to the invention have an IC₅₀ value as determined by one or more of the assays of less than 10 μM, more preferred of less than 1 μM, and even more preferred of less than 500 nM, such as of less than 100 nM.

Functional Assay I

The ability of the compounds to interact with the histamine H3 receptor as agonists, inverse agonists and/or antagonists, is determined by an in vitro functional assay utilizing membranes from HEK 293 cell expressing the human H3 receptors.

The H3 receptor is cloned by PCR and subcdoned into the pcDNA3 expression vector. Cells stably expressing the H3 receptor are generated by transfecting the H3-expression vectors into HEK 293 cells and using G418 to select for H3 clones. The human H3-HEK 293 clones are cultured in DMEM with glutamax, 10% foetal calf serum, 1% penicillin/streptavidin and 1 mg/ml G 418 at 37° C. and 5% CO₂.

The H3 receptor expressing cells are washed once with phosphate buffered saline (PBS) and harvested using versene (GIBCO-BRL). PBS is added and the cells are centrifuged for 5 min at 188 g. The cell pellet is resuspended in stimulation buffer to a concentration of 1×10⁶ cells/ml. cAMP accumulation is measured using the Flash Plate® cAMP assay (NEN• Life Science Products). The assay is generally performed as described by the manufacturer.

Briefly, 50 μl cell suspension is added to each well of the Flashplate which also contained 25 μl 40 μM isoprenaline, to stimulate cAMP generation, and 25 μl of test compound (either agonists or inverse agonists alone, or agonist and antagonist in combination). The assay can be run in “agonist-mode” which means that the test compound is added, in increasing concentration, on its own, to the cells, and cAMP is measured. If cAMP goes up, it is an inverse agonist; if cAMP does not change, it is a neutral antagonist, and if cAMP goes down, it is an agonist. The assay can also be run in the “antagonist-mode” which means that a test compound is added, in increasing concentrations, together with increasing concentrations of a known H3 agonist (eg RAMHA). If the compound is an antagonist, increasing concentrations of it cause a right-ward shift in the H3-agonist's dose-response curves. The final volume in each well is 100 μl. Test compounds are dissolved in DMSO and diluted in H₂O. The mixture is shaken for 5 min, and allowed to stand for 25 min at room temperature. The reaction is stopped with 100 μl “Detection Mix” per well. The plates are then sealed with plastic, shaken for 30 min, allowed to stand overnight, and finally the radioactivity is counted in the Cobra II auto gamma topcounter. EC₅₀ values are calculated by non-linear regression analysis of dose response curves (6 points minimum) using GraphPad Prism. Kb values are calculated by Schild plot analysis.

Functional Assay II

The ability of the compounds to bind and interact with the human H3 receptor as agonists, inverse agonists and/or antagonists, is determined by a functional assay, named [³⁵S] GTPγS assay. The assay measures the activation of G proteins by catalyzing the exchange of guanosine 5′-diphosphate (GDP) by guanosine 5′-triphosphate (GTP) at the α-subunit. The GTP-bounded G proteins dissociate into two subunits, Gα_{GTP} and Gβγ, which in turn regulate intracellular enzymes and ion channels. GTP is rapidly hydrolysed by the Gα-subunit (GTPases) and the G protein is deactivated and ready for a new GTP exchange cycle. To study the function of ligand induced G protein coupled receptor (GPCR) activation by an increase in guanine nucleotide exchange at the G proteins, the binding of [³⁵S]-guanosine-5′-O-(3-thio) triphosphate [³⁵S] GTPγS, a non-hydrolysed analogue of GTP, is determined. This process can be monitored in vitro by incubating cell membranes containing the G protein coupled receptor H3 with GDP and [³⁵S] GTPγS. Cell membranes are obtained from CHO cells stably expressing the human H3 receptor. The cells are washed twice in PBS, harvested with PBS+1 mM EDTA, pH 7.4 and centrifuged at 1000 rpm for 5 min. The cell pellet is homogenized in 10 ml ice-cold Hepes buffer (20 mM Hepes, 10 mM EDTA pH 7.4 (NaOH)) using an Ultra-Turrax homogenizer for 30 seconds and centrifuged for 15 min at 20.000 rpm. Following this centrifugation step, the membrane pellet is resuspended in 10 ml ice-cold Hepes buffer (20 mM Hepes, 0.1 mM EDTA pH 7.4 (NaOH)) and homogenized as describe above. This procedure is repeated twice except for the last homogenization step, the protein concentration is determined and membranes are diluted to a protein concentration at 2 mg/ml, aliquoted and kept at −80° C. until use.

In order to study the presence and the potency of an inverse agonist/antagonist the H3-receptor agonist ligand R-α-methyl histamine (RAMHA) is added. The ability of the test compound to counteract the effect of RAMHA is measured. When studying the effect of an agonist RAMHA is not added to the assay medium. The test compound is diluted in the assay buffer (20 mM HEPES, 120 mM NaCl, 10 mM MgCl₂ pH 7.4 (NaOH)) at various concentrations followed by addition of 10⁻⁸ nM RAMHA (only in the case where an inverse agonist/antagonist is examined), 3 μM GDP, 2.5 μg membranes, 0.5 mg SPA beads and 0.1 nM [³⁵] GTPγS and incubated for 2 hours by slightly shaking at room temperature. The plates are centrifuged at 1500 rpm for 10 min and the radioactivity is measured using a Top-counter. The results are analyzed by non linear regression and the IC₅₀ value is determined. RAMHA and other H3 agonists stimulate the binding of [³⁵S] GTPγS to membranes expressing the H3 receptor. In the antagonist/inverse agonist test, the ability of increasing amounts of test compound to inhibit the increased [³⁵S] GTPγS binding by 10⁻⁸ M RAMHA is measured as a decrease in radioactivity signal. The IC₅₀ value determined for an antagonist is the ability of this compound to inhibit the effect of 10⁻⁸M RAMHA by 50%. In the agonist test, the ability of increasing amounts of test compound is measured as an increase in radioactivity signal. The EC₅₀ value determined for an agonist, is the ability of this compound to increase the signal by 50% of the maximal signal that is obtained by 10⁻⁵ M RAMHA.

Preferably, the antagonists and agonists according to the invention have an IC₅₀/EC₅₀ value as determined by one or more of the assays of less than 10 μM, more preferred of less than 1 μM, and even more preferred of less than 500 nM, such as of less than 100 nM.

The Open Cage Schedule-Fed Rat Model

The ability of the present compounds to reduce weight is determined using the in vivo open cage Schedule-fed rat model.

Sprague-Dawley (SD) male rats of an age of about 1½ to 2 months and a weight of about 200-250 g are purchased from Mollegard Breeding and Research Centre A/S (Denmark). On arrival they are allowed some days of acclimatisation before being placed in individual open plastic cages. They are habituated to the presence of food (Altromin pelleted rat chow) in their home cage only during 7 hours in the morning from 07.30 to 14.30 all days a week. Water is present ad libitum. As the consumption of food has stabilised after 7 to 9 days, the animals are ready for use.

Each animal is used only once to avoid carry-over effects between treatments. During the test sessions, the test compound is administered intraperitoneally or orally 30 min before the start of the sessions. One group of animals is administered the test compound at different doses and a control group of animals is given a vehicle. Food and water intake are monitored at 1, 2 and 3 hours post administration.

Any side effects may rapidly be discovered (barrel-rolling, bushy fur etc.) since the animals are kept in transparent plastic cages to enable continuous monitoring.

1. A compound of the general formula (II): 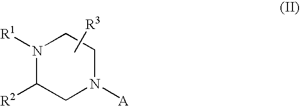
wherein
R² is hydrogen or C₁₋₄-alkyl,
(i) R¹ represents
branched C₄₋₆-alkyl, branched C₄₋₆-alkenyl or branched C₄₋₆-alkynyl with the proviso that R¹ is not isobutyl,
C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl,
R¹ and R² together form a C₃₋₆-alkylene bridge, and
A represents 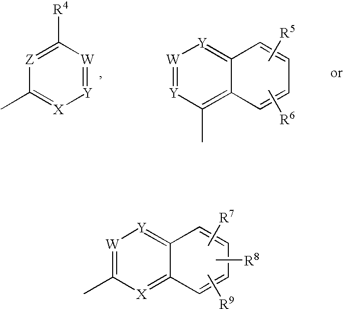
or
(ii) R¹ represents
ethyl, n-propyl or isopropyl,
R¹ and R² together form a C₃₋₆-alkylene bridge, and
A represents 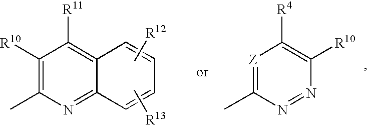
R³ is hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl, aryl-C₁₋₆-alkyl, amino, C₁₋₆-alkylamino, di-C₁₋₆-alkylamino, C₃₋₈-cycloalkyl, C₃₋₈-cycloalkyloxy, cyano, nitro, C₁₋₆-alkylsulfanyl, or C₁₋₆-alkylsulfonyl,
Z and X independently represent —N═, —C(H)═, —C(F)═, —C(Cl)═, —C(CN)═ or —C(CF₃)═,
W represents —N═ or —C(R¹⁰)═,
Y represents —N═ or —C(R¹¹)═,
R⁴, R⁵, R⁶, R⁷, R⁸, R⁹ R¹⁰, R¹¹, R¹² and R¹³ independently represent
hydrogen, halogen, hydroxy, trifluoromethyl, trifluoromethoxy, —SCF₃, amino, cyano, nitro, or —C(═O)NR¹⁴R¹⁵
C₁₋₁₀-alkyl, C₂₋₁₀-alkenyl, C₃₋₈-cycloalkyl, C₁₋₆-alkoxy, C₃₋₈-cycloalkyl-C₁₋₆-alkoxy, C₁₋₆-alkylamino, di-C₁₋₆-alkylamino, C₃₋₈-cycloalkyloxy, C₁₋₆-alkylsulfanyl, C₁₋₆-alkylsulfonyl, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶
aryl, aryl-C₁₋₆-alkyl, aryl-C₁₋₆-alkoxy or heteroaryl, which may optionally be substituted with one or more substituents selected from R¹⁷,
aroyl, heteroaroyl, aryloxy, heteroaryloxy, arylamino or heteroarylamino, which may optionally be substituted with one or more substituents selected from R¹⁸,
or two of R⁵, R⁶, R⁷, R⁸, R⁹, R¹⁰, R¹¹, R¹² and R¹³ in adjacent positions together form a C₁₋₆-alkylene bridge or an —O—C₁₋₆-alkylene-O— bridge,
R¹⁴ and R¹⁵ are independently hydrogen, C₁₋₆-alkyl, aryl-C₁₋₆-alkyl or R¹⁴ and R¹⁵ may together form a C₃₋₆-alkylene bridge
R¹⁶ is independently selected from aryl, heteroaryl, C₃₋₈-cycloalkyl, halogen, trifluoromethyl, trifluoromethoxy, NR¹⁹R²⁰ and C₁₋₆-alkoxy,
R¹⁷ is independently selected from halogen, hydroxy, trifluoromethyl, trifluoromethoxy, C₁₋₆-alkoxy, C₁₋₆-alkyl, amino, C₁₋₆-alkylsulfonyl, C₁₋₆-alkylamino, di-C₁₋₆-alkylamino, cyano, aryl, heteroaryl and C₃₋₈-cycloalkyl,
R¹⁸ is independently selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,
R¹⁹ and R²⁰ are independently hydrogen or C₁₋₆-alkyl, R₁₉ and R₂₀ may together form a C₃₋₆-alkylene bridge
with the proviso that the compound must not be 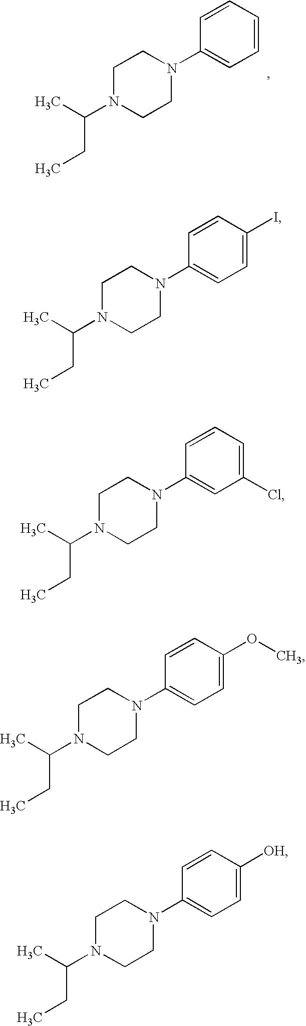 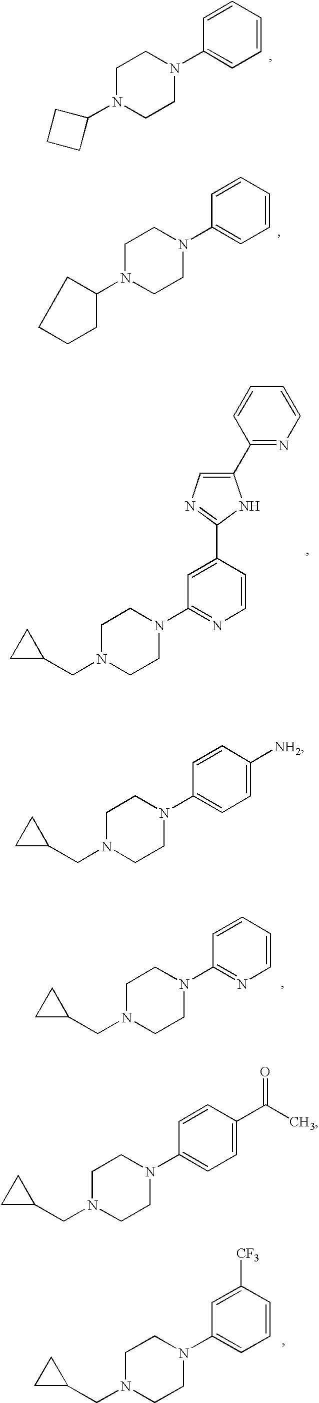 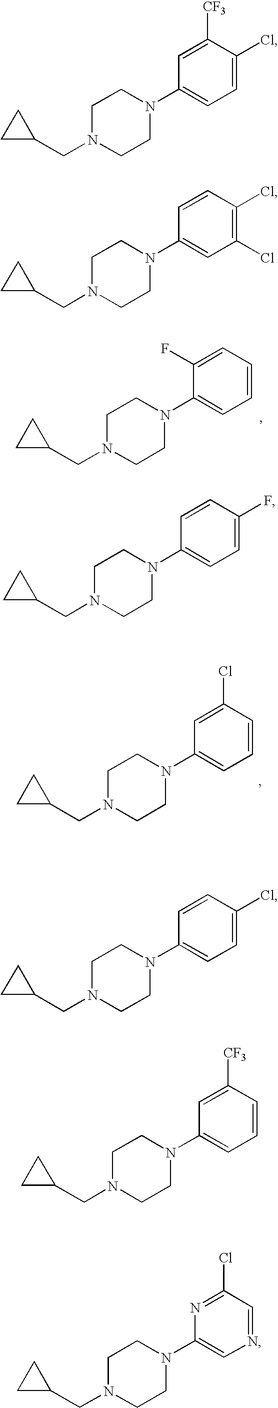 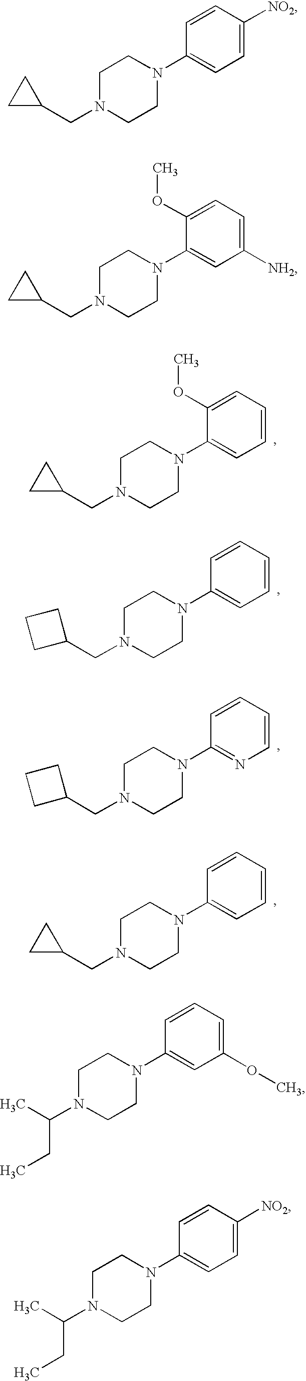 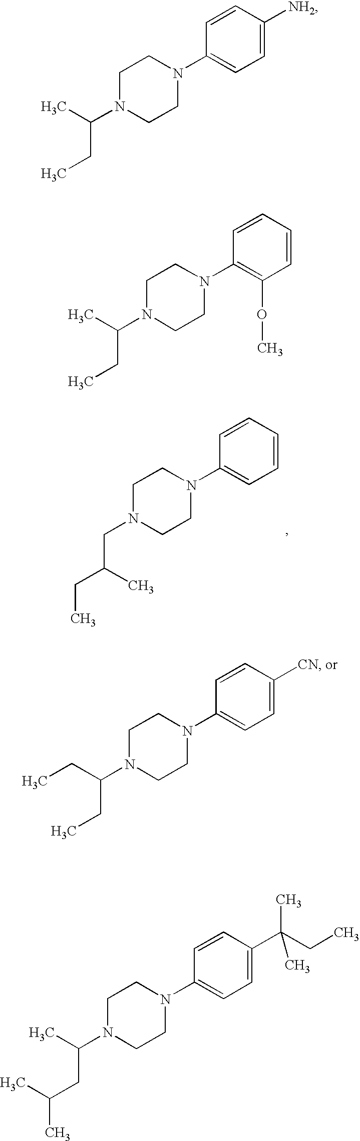
as well as any diastereomer or enantiomer or tautomeric form thereof including mixtures of these or a pharmaceutically acceptable salt thereof.

2. A compound according to claim 1, wherein R¹ is branched C₄₋₆-alkyl, C₃₋₅-cycloalkyl or C₃₋₆-cycloalkyl-C₁₋₃-alkyl with the proviso that R¹ is not isobutyl.

3. A compound according to claim 2, wherein R¹ is 1,1-(dimethyl)propyl, 1-ethylpropyl, cyclopropylmethyl, cyclopropyl, cyclobutyl, cyclopentyl or 1-cyclopropyl-1-methylethyl.

4. A compound according to claim 3, wherein R¹ is 1-ethylpropyl, cyclopropylmethyl, cyclopropyl or cyclopentyl.

5. A compound according to claim 1, wherein R¹ is branched C₄₋₆-alkyl or C₃₋₅-cycloalkyl with the proviso that R¹ is not isobutyl.

6. A compound according to claim 5, wherein R¹ is 1-ethylpropyl, cyclopropyl or cyclopentyl.

7. A compound according to any one of the claims 1 to 6, wherein Z is —C(H)═, —N═, or —C(F)═.

8. A compound according to claim 7 wherein Z is —C(H)═ or —N═.

9. A compound according to claim 8 wherein Z is —C(H)═.

10. A compound according to claim 8 wherein Z is —N═.

11. A compound according to any one of the claims 1 to 10 wherein X is —C(H)═, —N═, or —C(F)═.

12. A compound according to claim 11 wherein Z is —C(H)═ or —N═.

13. A compound according to claim 12 wherein Z is —C(H)═.

14. A compound according to claim 12 wherein Z is —N═.

15. A compound according to any one of the claims 1 to 14 wherein W is —N═.

16. A compound according to any one of the claims 1 to 14 wherein W is —C(R¹⁰)═.

17. A compound according to any one of the claims 1 to 16 wherein Y is —N═.

18. A compound according to any one of the claims 1 to 16 wherein Y is —C(R¹¹)═.

19. A compound according to any one of the claims 1 to 18 wherein R² is hydrogen.

20. A compound according to any one of the claims 1 to 18 wherein R² is C₁₋₄-alkyl.

21. A compound according to claim 20 wherein R² is methyl or ethyl.

22. A compound according to claim 1 of the general formula (III) 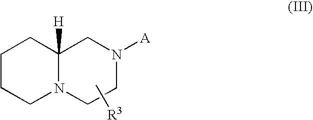
wherein A and R³ are as defined in claim 1.

23. A compound according to any one of the claims 1 to 22 wherein R³ is hydrogen, halogen, hydroxy, trifluoromethyl, trifluoromethoxy, C₁₋₁₀-alkyl, C₁₋₆-alkoxy, aryl, aryl-C₁₋₆-alkyl, amino, C₃₋₈-cycloalkyl, C₃₋₈-cycloalkyloxy, cyano or nitro.

24. A compound according to claim 23 wherein R³ is hydrogen, halogen, hydroxy, trifluoromethyl, C₁₋₁₀-alkyl, C₁₋₆-alkoxy, cyano or nitro.

25. A compound according to claim 24 wherein R³ is hydrogen, halogen, hydroxy, trifluoromethyl, C₁₋₆-alkyl, or cyano.

26. A compound according to claim 25 wherein R³ is hydrogen, halogen, or C₁₋₆-alkyl.

27. A compound according to claim 25 wherein R³ is hydrogen or methyl.

28. A compound according to any one of the claims 1 to 27 wherein R⁴, R⁵, R⁶, R⁷, R⁸, and R⁹ independently represent
hydrogen, halogen, hydroxy, trifluoromethyl, trifluoromethoxy, —SCF₃, amino, or cyano,
C₁₋₁₀-alkyl, C₃₋₈-cycloalkyl, C₁₋₆-alkoxy, C₃₋₈-cycloalkyloxy, C₂₋₁₀-alkanoyl, cycloalkanoyl, C₃₋₈-heterocyclyl or C₄₋₉-heterocycloalkanoyl, which may optionally be substituted with one or more substituents selected from R¹⁶
aryl, aryl-C₁₋₆-alkyl, aryl-C₁₋₆-alkoxy or heteroaryl, which may optionally be substituted with one or more substituents selected from R¹⁷,
aroyl, heteroaroyl, aryloxy, heteroaryloxy, which may optionally be substituted with one or more substituents selected from R¹⁸,
or two of R⁵, R⁶, R⁷, R⁸, R⁹ in adjacent positions together form a C₁₋₆-alkylene bridge or an —O—C₁₋₆-alkyl-O— bridge.

29. A compound according to claim 28 wherein R⁴, R⁵, R⁶, R⁷, R⁸, and R⁹ independently represent
hydrogen, halogen, hydroxy, trifluoromethyl, trifluoromethoxy, —SCF₃, or cyano,
C₁₋₁₀-alkyl, C₁₋₆-alkoxy, C₃₋₈-cycloalkyloxy, which may optionally be substituted with one or more substituents selected from R¹⁶
Aryl or aryl-C₁₋₆-alkyl, which may optionally be substituted with one or more substituents selected from R¹⁷,
Aroyl or aryloxy, which may optionally be substituted with one or more substituents selected from R¹⁸,
or two of R⁵, R⁶, R⁷, R⁸, R⁹ in adjacent positions together form a C₁₋₆-alkylene bridge or an —O—C₁₋₆-alkyl-O— bridge.

30. A compound according to claim 28 wherein R⁴, R⁵, R⁶, R⁷, R⁸, and R⁹ independently represent
hydrogen, halogen, or cyano,
C₁₋₁₀-alkyl or C₁₋₆-alkoxy which may optionally be substituted with one or more substituents selected from R¹⁶
Aryl optionally substituted with one or more substituents selected from R¹⁷,
Aroyl or aryloxy, which may optionally be substituted with one or more substituents selected from R¹⁸,
or two of R⁵, R⁶, R⁷, R⁸, R⁹ in adjacent positions together form a C₁₋₆-alkylene bridge or an —O—C₁₋₆-alkyl-O— bridge.

31. A compound according to claim 30 wherein C₁₋₁₀-alkyl represents methyl, ethyl, propyl, or isopropyl.

32. A compound according to claim 30 wherein C₁₋₆-alkoxy represents methoxy, ethoxy or propoxy.

33. A compound according to claim 32 wherein C₁₋₆-alkoxy represents methoxy.

34. A compound according to claim 30 wherein aryl represents phenyl.

35. A compound according to claim 30 wherein aroyl represents —C(═O)-phenyl.

36. A compound according to claim 30 wherein aryloxy represents —O-phenyl.

37. A compound according to claim 1, wherein R¹ is ethyl or isopropyl.

38. A compound according to claim 37 wherein R¹ is isopropyl.

39. A compound according to claim 37 wherein R¹ is ethyl

40. A compound according to claim 39 wherein R¹ and R² together form a C₃₋₄-alkylene bridge.

41. A compound according to any one of the claims 37 to 40 wherein R¹⁰, R¹¹, R¹² and R¹³ independently represent
hydrogen, halogen, hydroxy, trifluoromethyl, trifluoromethoxy, cyano, or —C(═O)NR¹⁴R¹⁵
C₁₋₁₀-alkyl, C₃₋₈cycloalkyl, C₁₋₆-alkoxy, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶
aryl, aryl-C₁₋₆-alkyl, aryl-C₁₋₆-alkoxy or heteroaryl, which may optionally be substituted with one or more substituents selected from R¹⁷,
aroyl optionally substituted with one or more substituents selected from R¹⁸,
or two of R¹⁰, R¹¹, R¹² and R¹³ in adjacent positions together form a C₁₋₆-alkylene bridge.

42. A compound according to claim 41 wherein R¹⁰, R¹¹, R¹² and R¹³ independently represent
hydrogen, halogen, hydroxy, trifluoromethyl, trifluoromethoxy, cyano, or —C(═O)NR¹⁴R¹⁵
C₁₋₁₀-alkyl, C₃₋₈-cycloalkyl, C₁₋₆-alkoxy, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶
aryl, aryl-C₁₋₆-alkyl, aryl-C₁₋₆-alkoxy or heteroaryl, which may optionally be substituted with one or more substituents selected from R¹⁷,
aroyl optionally substituted with one or more substituents selected from R¹⁸,
or two of R¹⁰, R¹¹, R¹² and R¹³ in adjacent positions together form a C₁₋₆-alkylene bridge.

43. A compound according to claim 42 wherein R¹⁰, R¹¹, R¹² and R¹³ independently represent
hydrogen, halogen, trifluoromethyl, or —C(═O)NR¹⁴R¹⁵
C₁₋₁₀-alkyl, C₁₋₆-alkoxy, C₂₋₁₀-alkanoyl, C₄₋₉-cycloalkanoyl, C₄₋₉-heterocycloalkanoy C₄₋₉-heterocycloalkoxy, which may optionally be substituted with one or more substituents selected from R¹⁶
aryl, aryl-C₁₋₆-alkyl, or aryl-C₁₋₆-alkoxy, which may optionally be substituted with one or more substituents selected from R¹⁷,
aroyl optionally substituted with one or more substituents selected from R¹⁸,
or two of R¹⁰, R¹¹, R¹² and R¹³ in adjacent positions together form a C₁₋₆-alkylene bridge.

44. A compound according to claim 43 wherein R¹⁰, R¹¹, R¹² and R¹³ independently represent
hydrogen, halogen, trifluoromethyl, or —C(═O)NR¹⁴R¹⁵
C₁₋₁₀-alkyl or C₄₋₉-heterocycloalkanoyl, which may optionally be substituted with one or more substituents selected from R¹⁶
aryl optionally substituted with one or more substituents selected from R¹⁷,
aroyl optionally substituted with one or more substituents selected from R¹⁸,
or two of R¹⁰, R¹¹, R¹² and R¹³ in adjacent positions together form a C₁₋₆-alkylene bridge.

45. A compound according to claim 44 wherein C₁₋₁₀-alkyl represents methyl, ethyl, or propyl.

46. A compound according to claim 44 wherein C₄₋₉-heterocycloalkanoyl represents piperidin-alkanoyl or pyrrolidin-alkanoyl.

47. A compound according to claim 44 wherein aryl represents phenyl

48. A compound according to any one of the claims 1 to 47 wherein R¹⁴ and R¹⁵ are independently methyl, ethyl or benzyl.

49. A compound according to any one of the claims 1 to 48 wherein R¹⁶ is halogen, trifluoromethyl, trifluoromethoxy and C₁₋₆-alkoxy.

50. A compound according to any one of the claims 1 to 49 wherein R¹⁷ is halogen, hydroxy, trifluoromethyl, C₁₋₆-alkoxy, C₁₋₆-alkyl, C₁₋₆-alkylsulfonyl, or cyano.

51. A compound according to claim 50 wherein R¹⁷ is halogen, trifluoromethyl, C₁₋₆-alkoxy or C₁₋₆-alkylsulfonyl.

52. A compound according to any one of the claims 1 to 51 wherein R¹⁸ is C₁₋₁₀-alkyl, halogen, trifluoromethyl, C₁₋₆-alkoxy, cyano, amino and hydroxy.

53. A compound according to claim 52 wherein R¹⁸ is halogen, C₁₋₆-alkoxy and hydroxy.

54. Use of a compound according to any one of the preceding claims 1 to 53 as a pharmaceutical composition.

55. A pharmaceutical composition comprising, as an active ingredient, at least one compound according to any one of the claims 1 to 53 together with one or more pharmaceutically acceptable carriers or excipients.

56. A pharmaceutical composition according to claim 55 in unit dosage form, comprising from about 0.05 mg to about 1000 mg, preferably from about 0.1 mg to about 500 mg and especially preferred from about 0.5 mg to about 200 mg of the compound according to any one of the claims 1 to 53.

57. Use of a compound of the general formula (II′): 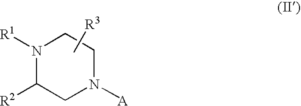
wherein
R² is hydrogen or C₁₋₄-alkyl,
R¹ represents
C₁₋₈-alkyl, C₂₋₈-alkenyl or C₂₋₈-alkynyl, which may optionally be substituted with one or more halogen substituents,
C₃₋₅-cycloalkyl, C₃₋₇-cycloalkenyl, C₃₋₆-cycloalkyl-C₁₋₃-alkyl or C₃₋₆-cycloalkenyl-C₁₋₃-alkyl, which may optionally be substituted with one or more halogen substituents,
R¹ and R² together form a C₃₋₆-alkylene bridge,
A represents 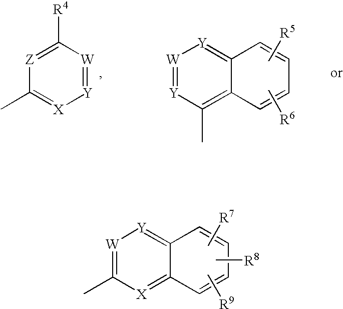
R³ is hydrogen, halogen, hydroxy, trifluoromethyl, trifluoromethoxy, C₁₋₁₀-alkyl, C₂₋₁₀-alkenyl, C₃₋₈-cycloalkyl, C₁₋₆-alkoxy, aryl, aryl-C₁₋₆-alkyl, amino, C₁₋₆-alkylamino, di-C₁₋₆-alkylamino, C₃₋₈-cycloalkyl, C₃₋₈-cycloalkyloxy, cyano, nitro, C₁₋₆-alkylsulfanyl, or C₁₋₆-alkylsulfonyl,
Z and X independently represent —N═, —C(H)═, —C(F)═, —C(Cl)═, —C(CN)═ or —C(CF₃)═,
W represents —N═ or —C(R¹⁰)═,
Y represents —N═ or —C(R¹¹)═,
R⁴, R⁵, R⁶, R⁷, R⁸, R⁹ R¹⁰, R¹¹, R¹² and R¹³ independently represent
hydrogen, halogen, hydroxy, trifluorom ethyl, trifluoromethoxy, —SCF₃, amino, cyano, nitro, or —C(═O)NR¹⁴R¹⁵
C₁₋₁₀-alkyl, C₂₋₁₀-alkenyl, C₃₋₈-cycloalkyl, C₁₋₆-alkoxy, C₃₋₈-cycloalkyl-C₁₋₆-alkoxy, C₁₋₆-alkylamino, di-C₁₋₆-alkylamino, C₃₋₈-cycloalkyloxy, C₁₋₆-alkylsulfanyl, C₁₋₆-alkylsulfonyl, C₂₋₁₀-alkanoyl, C₄₋₉-cycloalkanoyl, C₃₋₈-heterocyclyl or C₄₋₉-heterocycloalkanoyl, C₄₋₉-heterocycloalkoxy, which may optionally be substituted with one or more substituents selected from R¹⁶
aryl, aryl-C₁₋₆-alkyl, aryl-C₁₋₆-alkoxy or heteroaryl, which may optionally be substituted with one or more substituents selected from R¹⁷,
aroyl, heteroaroyl, aryloxy, heteroaryloxy, arylamino or heteroarylamino, which may optionally be substituted with one or more substituents selected from R¹⁸,
or two of R⁵, R⁶, R⁷, R⁸, R⁹, R¹⁰, R¹¹, R¹² and R¹³ in adjacent positions together from a C₁₋₆-alkylene bridge or an —O—C₁₋₆-alkylene-O— bridge,
R¹⁴ and R¹⁵ are independently hydrogen, C₁₋₆-alkyl, aryl-C₁₋₆-alkyl or R¹⁴ and R¹⁵ may together form a C₃₋₆-alkylene bridge
R¹⁶ is independently selected from aryl, heteroaryl, C₃₋₈-cycloalkyl, halogen, trifluoromethyl, trifluoromethoxy, NR¹⁹R²⁰ and C₁₋₆-alkoxy,
R¹⁷ is independently selected from halogen, hydroxy, trifluoromethyl, trifluoromethoxy, C₁₋₆-alkoxy, C₁₋₆-alkyl, amino, C₁₋₆-alkylsulfonyl, C₁₋₆-alkylamino, di-C₁₋₆-alkylamino, cyano, aryl, heteroaryl and C₃₋₈-cycloalkyl,
R¹⁸ is independently selected from aryl, heteroaryl, C₁₋₁₀-alkyl, C₃₋₈-cycloalkyl, halogen, trifluoromethyl, trifluoromethoxy, C₁₋₆-alkoxy, cyano, amino, C₁₋₆-alkylamino, di-C₁₋₆-alkylamino and hydroxy,
R¹⁹ and R²⁰ are independently hydrogen or C₁₋₆-alkyl, R¹⁹ and R₂₀ may together form a C₃₋₆-alkylene bridge,
as well as any diastereomer or enantiomer or tautomeric form thereof including mixtures of these or a pharmaceutically acceptable salt thereof for the preparation of a pharmaceutical composition for the treatment of disorders and diseases related to the histamine H3 receptor.

58. Use of a compound as defined in claim 57 for the preparation of a pharmaceutical composition for the treatment of diseases and disorders in which an inhibition of the H3 histamine receptor has a beneficial effect.

59. Use of a compound as defined in claim 57 for the preparation of a pharmaceutical composition having histamine H3 antagonistic activity or histamine H3 inverse agonistic activity.

60. Use of a compound as defined in claim 57 for the preparation of a pharmaceutical composition for the reduction of weight.

61. Use of a compound as defined in claim 57 for the preparation of a pharmaceutical composition for the treatment of overweight or obesity.

62. Use of a compound as defined in claim 57 for the preparation of a pharmaceutical composition for the suppression of appetite or for satiety induction.

63. Use of a compound as defined in claim 57 for the preparation of a pharmaceutical composition for the prevention and/or treatment of disorders and diseases related to overweight or obesity.

64. Use of a compound as defined in claim 57 for the preparation of a pharmaceutical composition for the prevention and/or treatment of eating disorders such as bulimia and binge eating.

65. Use of a compound as defined in claim 57 for the preparation of a pharmaceutical composition position for the treatment of IGT.

66. Use of a compound as defined in claim 57 for the preparation of a pharmaceutical composition position for the treatment of type 2 diabetes.

67. Use of a compound as defined in claim 57 for the preparation of a pharmaceutical composition position for the delaying or prevention of the progression from IGT to type 2 diabetes.

68. Use of a compound as defined in claim 57 for the preparation of a pharmaceutical composition position for the delaying or prevention of the progression from non-insulin requiring type 2 diabetes to insulin requiring type 2 diabetes.

69. Use of a compound as defined in claim 57 for the preparation of a pharmaceutical composition position for the treatment of diseases and disorders in which a stimulation of the H3 histamine receptor has a beneficial effect.

70. Use of a compound as defined in claim 57 for the preparation of a pharmaceutical composition position having histamine H3 agonistic activity.

71. Use of a compound as defined in claim 57 for the preparation of a pharmaceutical composition position for the treatment of allergic rhinitis, ulcer or anorexia.

72. Use of a compound as defined in claim 57 for the preparation of a pharmaceutical composition position for the treatment of Alzheimer's disease, narcolepsy or attention deficit disorders.

73. A method for the treatment of disorders or diseases related to the H3 histamine receptor the method comprising administering to a subject in need thereof an effective amount of a compound as defined in claim 57 or a pharmaceutical composition according to claim 55 or 56.

74. The method according to claim 73 wherein the effective amount of the compound is in the range of from about 0.05 mg to about 2000 mg, preferably from about 0.1 mg to about 1000 mg and especially preferred from about 0.5 mg to about 500 mg per day.
